(19) United States
(12) Patent Application Publication
(10) Pub. No.: US 2024/0384245 A1
(43) Pub. Date: Nov. 21, 2024

(54) METHOD FOR TREATING A DISEASE

(71) Applicant: SIRT6 LTD, Ness Ziona (IL)

(72) Inventors: Hagit ASHUSH, Ashkelon (IL); Boaz MISHOLI, Tel Aviv-Yafo (IL); Yaki EIDELSTEIN, Rehovot (IL); Haim COHEN, Modiin-Maccabim-Reut (IL)

(73) Assignee: SIRT6 LTD, Ness Ziona (IL)

(21) Appl. No.: 18/696,787

(22) PCT Filed: Sep. 29, 2022
(86) PCT No.: PCT/IL2022/051037

§ 371 Date: Mar. 28, 2024

Related U.S. Application Data
(60) Provisional application No. 63/261,805, filed on Sep. 29, 2021.
(60) Provisional application No. 63/266,010, filed on Dec. 27, 2021.
(60) Provisional application No. 63/268,629, filed on Feb. 28, 2022.

(51) Int. Cl. C12N 9/10; A61K 9/51; A61K 38/00; A61K 48/00; A61P 1/16; C12N 15/113; C12N 15/67; C12N 15/86; C12N 15/88

(52) U.S. Cl. CPC C12N 9/1029; A61K 9/5123; A61K 48/0066; A61P 1/16; C12N 15/113; C12N 15/67; C12N 15/86; C12N 15/88; A61K 38/00; C12N 2310/113; C12N 2750/14143; C12N 2830/50

(57) ABSTRACT

A novel mRNA molecule encoding SIRT6, methods and uses thereof for treating diseases including fatty liver diseases, e.g., NASH.

### TECHNOLOGICAL FIELD

The present disclosure concerns therapeutic methods based on increasing the expression of SiRT6 using mRNA molecules.

### BACKGROUND ART

References considered to be relevant as background to the presently disclosed subject matter are listed below:
1. Chiba et al., PLOS ONE 11 (10), 2016 DOI: 10.1371/journal.pone.0164191.
2. den Boer M. et al. Arterioscler. Thromb. Vasc. Biol. 24:644-649, 2004.
3. Desantis V., et al Haematologica 103, 1-4 (2017)
4. Hou et al Nature Reviews Materials 6, 1078-1094 (2021)
5. Jung, E. S et al., Sci. Rep. 6:25628. doi: 10.1038 (2016)
6. Ka S. O. et al. The FASEB Journal. Vol. 31(9):3999-4010, 2017
7. Kaluski S et al., Cell Rep. 18, 3052-3062. (2017)
8. Kim H S. et al. Cell Metabolism 12, 224-236, (2010).
9. Kong, Junsheng Ye et al. Cell Rep. December 26; 5(6): 1639-1649 (2013)
10. Lerrer B et al, Carcinogenesis. 37(2):108-118 (2016)
11. Liu Z et al., Transl. Res. 172, 18-29 (2016)
12. Maity S. et al. J Biol Chem 295(2):415-34, (2020).
13. Masato Fujii et al, Med Mol Morphol 46:141-152 (2013).
14. Mayr C. Cold Spring Harb. Perspect. Biol., 11 (2019), p. a034728
15. Postic C. and Girard J. J. Clin. Invest. 118:829-838, (2008).
16. Roichman A. et al., Nature Communications 12, Article no: 3208 (2021)
17. Ronnebaum et al., Mol. and Cellular Biology 33(22) 4461-4472 (2013).
18. Sundaresan N. R et al., Nat. Med. 18, 1643-1650 (2012).
19. Tao R. et al. Journal of Lipid Research 54(10): 2745-2753, (2013).
20. Tasselli et al Trends Endocrinol. Metab. 28(3): 168-185 (2017).
21. Umadevi Thirumurthi et al., Sci Signal; 7(336), (2014)
22. Wang et al Scientific Reports 11 Article No. 14253 (2021)
23. Winter J. et al. Nat Cell Biol. 11:228-34 (2009).
24. Xiao C. et al. Journal of Biological Chemistry 287 (50): 41903-41913 (2012).
25. Xiong X., Diabetologia 59, 151-160 (2016)
26. Yu L Metal., J. Pineal Res. 70: e12698 (2021)
27. Zhong L et al., Cell 140, 280-293 (2010)
28. Zhong, X. et al. Cell Mol Gastroenterol Hepatol 10(2): 341-364, (2020).

Acknowledgement of the above references herein is not to be inferred as meaning that these are in any way relevant to the patentability of the presently disclosed subject matter.

### BACKGROUND

SIRT6 is a member of the evolutionarily conserved sirtuin family of NAD (+)-dependent protein deacetylases. SIRT6 has been implicated in multiple biological processes including glucose and lipid metabolism, inflammation, anti-oxidative stress, DNA repair, fibrosis, and tumor suppression.

SIRT6 promotes hepatic triglyceride (TG) homeostasis through inhibition of de novo lipogenesis and activation of fatty acid oxidation by histone H3 deacetylation. The absence of SIRT6 results in accumulation of TG, which is associated with fatty liver (den Boer et al., 2004, Postic and Girard, 2008). Hepatic-specific disruption of SIRT6 in mice results in fatty liver formation due to enhanced glycolysis and triglyceride synthesis (Kim HS, 2010).

SIRT6 also inhibits cholesterol biosynthesis by repression of the master regulator SREBP2 and its target genes. Hepatic SIRT6 deficiency leads to elevated cholesterol and triglycerides in the blood and liver as well (Tao R et al, 2013).

Hepatocyte-specific sirtuin 6 deletion predisposes to nonalcoholic steatohepatitis by up-regulation of Bach1, an Nrf2 repressor (Ka et al, 2017).

SIRT6 deficiency transcriptionally up-regulates TGF-beta signaling and induces fibrosis in mice (Maity S et al, 2020). SIRT6 knockout mice develop fibrosis in the heart, liver, kidneys, and lungs (Xiao al, 2012).

SIRT6 has been implicated in fatty liver disease and was found to protect against liver fibrosis by deacetylation and suppression of SMAD3 in hepatic Stellate cells (Zhong et al, 2020).

SIRT6 is involved in many kinds of aging related diseases. In Alzheimer's disease (AD) patients, SIRT6 plays AD—protective function via maintaining genomic stability and preventing DNA damage in brain (Jung et al., 2016; Kaluski et al., 2017). In the context of cancer, SIRT6 was shown to have a role as a tumor suppressor in many types of tumors by protecting against tumor growth through the functions of controlling DNA damage repair, genomic stability, cellular metabolic homeostasis, and apoptosis (Desantis et al., 2017).

SIRT6 modulates either directly or indirectly key processes linked to cell fate determination and oncogenesis and prevents genomic instability, maintains telomere integrity, and regulates metabolic homeostasis and DNA repair (Lerrer et al., 2016). Through these functions, SIRT6 protects, among others, against cancer initiation and progression (for a review, see Tasselli et al, 2017).

In the cardiovascular system, SIRT6 plays a protective function by improving vascular endothelial dysfunction to some extent, delaying the formation of atherosclerotic plaques, and inhibiting cardiac hypertrophy and heart failure (Sundaresan et al., 2012; Liu et al., 2016). In addition, several studies showed that SIRT6 is a principal regulator of glucose metabolism homeostasis (Zhong et al., 2010; Xiong et al., 2016) that can attenuate diabetic cardiomyopathy and reduce myocardial vulnerability to ischemia-reperfusion injury in diabetic patients (Yu et al., 2021). SIRT6 overexpression leads to a lifespan extension and reduction in frailty in both male and female B6 mice. SIRT6 -transgenic mice preserve hepatic glucose output and glucose homeostasis through an improvement in the utilization of two major gluconeogenic precursors, lactate and glycerol emphasize the role of SIRT6 in energy homeostasis in old age to delay frailty and preserve healthy aging (Roichman et al., 2021).

WO 2013/151736 describes methods for in vivo production of proteins by administering a formulation comprising a modified mRNA molecule. Among others, WO 2013/151736 describes the production of SIRT6 in splenocytes of mice intravenously injected with lipoplexed sirtuin 6 (SIRT6 ) mRNA.

### GENERAL DESCRIPTION

In one aspect, the present invention provides a non-native, isolated nucleic acid molecule encoding SIRT6, wherein said nucleic acid molecule comprises an mRNA molecule encoding SIRT6, a 3′ untranslated region (3′UTR), and a 5′ untranslated region (5′UTR).

In one embodiment, said mRNA molecule comprises the nucleic acid sequence transcribed from SEQ ID NO: 11, or a variant thereof having at least 75%, 80%, 85%, 90%, 95% or 99% sequence identity with SEQ ID NO: 11.

In another embodiment, said mRNA molecule encoding SIRT6 comprises a codon optimized nucleic acid sequence.

In one embodiment, said codon optimized nucleic acid sequence comprises any one of the nucleic acid sequences transcribed from SEQ ID NOs: 41-49.

In a specific embodiment, said codon optimized nucleic acid sequence comprises the nucleic acid sequence transcribed from SEQ ID NO: 48.

In one embodiment, said 3′UTR is the native 3′UTR sequence of the SIRT6 mRNA.

In another embodiment, the 3′UTR sequence is modified.

In one embodiment, said modified 3′UTR comprises a deletion or an addition such that one or more miRNA binding sites are eliminated or introduced to the nucleic acid molecule.

In one embodiment, said native 3′UTR sequence of the SIRT6 mRNA is substituted with a heterologous 3′UTR sequence.

In one embodiment, said 3′UTR comprises human alpha globin gene 3′UTR.

In one embodiment, said 5′UTR is the native 5′UTR sequence of the SIRT6 mRNA.

In another embodiment, said 5′UTR sequence is modified.

In one embodiment, said native 5′UTR sequence of the SIRT6 mRNA is substituted with a heterologous 5′UTR sequence.

In one embodiment, said 5′UTR is a nucleic acid sequence selected from the group consisting of SEQ ID Nos: 54-56.

In a specific embodiment, said 5′ UTR nucleic acid sequence is SEQ ID No: 54.

In another specific embodiment, said 5′ UTR nucleic acid sequence is a 5′UTR used in expression vectors for expression of viral genes, e.g., SEQ ID No: 56.

In one embodiment, said isolated nucleic acid molecule further comprises a 3′ end having at least 40 consecutive adenosine (A) nucleotides, at least 80 consecutive adenosine (A) nucleotides, at least 100 consecutive adenosine (A) nucleotides, at least 120 consecutive adenosine (A) nucleotides, or about 120 consecutive adenosine (A) nucleotides.

In another embodiment, said isolated nucleic acid molecule further comprises a 5′CAP.

In one embodiment, said mRNA molecule encoding SIRT6 is a modified SIRT6 mRNA.

In one embodiment, the uridines of the mRNA molecule are substituted with N1-Methylpseudouridine-5′-Triphosphate.

In one embodiment, said modified SIRT6 mRNA comprises one or more mutations that prevent phosphorylation and/or ubiquitination of the SIRT6 protein.

In some embodiments, said one or more mutations is at the amino acid residue S338 (Ser338) of the SIRT6 protein.

In some embodiments, said one or more mutations comprises substituting the arginine (Arg, R) and/or leucine (Leu, L) amino acid residues within the RXXL motif of the first D-box-activating domain of the SIRT6 protein with alanine (Ala, A).

In one embodiment, said substituting comprises a substitution from RVGL to AVGA at positions 103-106.

In one embodiment, said one or more mutations is in the C-terminal site of SIRT6, between amino acid residues positions 263-334.

In one embodiment, said one or more mutations is at the amino acid residue K170 (Lys 170) of the SIRT6 protein.

In one embodiment, said mutation is a substitution of lysine to arginine (K170R).

In one embodiment, said isolated nucleic acid molecule is encapsulated in lipid nanoparticles (LNP).

In one embodiment, said LNP comprise D-Lin-MC3-DMA.

In another embodiment, said LNP comprise Lipid 5.

In another aspect, the present invention provides a method for treating a disease or disorder, for reducing frailty or for increasing longevity, in a subject in need thereof comprising a step of administering an isolated nucleic acid molecule encoding SIRT6 to said subject wherein said disease or disorder is at least one of a fibrotic condition, a fibrosis-associated condition, a metabolic disorder, a liver disease, a kidney disease, or cancer.

In another aspect, the present invention provides a method for treating a disease or disorder, for reducing frailty or for increasing longevity, in a subject in need thereof comprising a step of administering the isolated nucleic acid molecule of the invention to said subject wherein said disease or disorder is at least one of a fibrotic condition, a fibrosis-associated condition, a metabolic disorder, a liver disease, a kidney disease, or cancer.

In another aspect, the present invention provides an isolated nucleic acid molecule encoding SIRT6 for use in a method of treating a disease or disorder, for reducing frailty, or for increasing longevity, in a subject in need thereof wherein said method comprises a step of administering a therapeutically effective amount of the isolated nucleic acid molecule, wherein said disease or disorder is at least one of a fibrotic condition, a fibrosis-associated condition, a metabolic disorder, a liver disease, a kidney disease, or cancer.

In another aspect, the present invention provides a pharmaceutical composition comprising an isolated nucleic acid molecule encoding SIRT6 and a suitable carrier or excipient for use in a method of treating a disease or disorder, for reducing frailty, or for increasing longevity, in a subject in need thereof wherein said method comprises a step of administering a therapeutically effective amount of the isolated nucleic acid molecule, wherein said disease or disorder is at least one of a fibrotic condition, a fibrosis-associated condition, a metabolic disorder, a liver disease, a kidney disease, or cancer.

In one embodiment, said fibrotic condition is selected from a group consisting of radiation-induced fibrosis, cardiac fibrosis, pulmonary fibrosis, liver fibrosis, skin fibrosis, fibrotic conditions in the eye, brain fibrosis, and kidney fibrosis.

In one embodiment, said fibrosis-associated conditions are inflammation, endothelial or epithelial to mesenchymal transition, parenchymal injury, scarring, or cirrhosis.

In one embodiment, said liver disease is NAFLD.

In one embodiment, said NAFLD is nonalcoholic steatohepatitis (NASH).

In one embodiment, said kidney disease is chronic kidney disease (CKD).

In another aspect, the present invention provides a method for reducing liver fat accumulation, liver fibrosis and/or liver inflammation in a subject in need thereof comprising a step of administering the isolated nucleic acid molecule of the invention to said subject.

In another aspect, the present invention provides a method for reducing glucose, fat and/or triglyceride levels in the serum of a subject in need thereof comprising a step of administering the isolated nucleic acid molecule of the invention to said subject.

In one embodiment, said subject is a human subject.

In one embodiment, the methods of the invention further comprise measuring the levels of SIRT6 mRNA expression or the levels of SIRT6 protein in said subject, prior to and/or during treatment, wherein the administered amount of said isolated nucleic acid molecule is adjusted according to the measured levels of SIRT6.

In one embodiment, the methods of the invention further comprise administering to said subject an additional therapeutic agent.

In one embodiment, said additional therapeutic agent is an anti-miR molecule that targets a miR known to negatively regulate SIRT6 expression.

In some embodiments, said anti-miR molecule is selected form the group consisting of Anti-miR-370, Anti-miR-34c-5p, Anti-miR-351-5p, Anti-miR-378b, Anti miR-186, Anti-miR-34a, Anti-miR-125b, Anti-miR-495, Anti-miR-25, Anti-miR-338-3p, Anti-miR-92a-3p, Anti-miR-766-3p, Anti-miR-33a-5p, Anti-miR-33b, Anti-miR-10396b-5p, Anti-miR-6787-5p, Anti-miR-137-3p, Anti-miR-1908-5p, Anti-miR-663a, Anti-miR-541-3p, Anti-miR-654-5p, and Anti-miR-122-5p.

In one embodiment, said additional therapeutic agent is an adeno associated virus (AAV) comprising SiRT6 gene.

In a specific embodiment, the methods of the invention comprise administering a single dose of the AAV followed by multiple administrations of the isolated nucleic acid molecule.

### BRIEF DESCRIPTION OF THE DRAWINGS

To exemplify and better understand the subject matter that is disclosed herein, embodiments will now be described, by way of non-limiting example only, with reference to the accompanying drawings, in which:

FIGS. 1A-1B present expression of mouse SIRT6 in mSIRT6 -transfected HEK293 cells. (A) SIRT6 mRNA. Results are presented as fold of Control untreated (without transfection) cells. The graph shows three doses of transfected SIRT6 mRNA, and negative controls of Lipofectamine MessengerMAX™ reagent only (Lipo Only) and control untreated cells (B) Protein levels of mouse SIRT6 expressed in HEK293 cells, endogenous human SIRT6, and Tubulin.

FIGS. 2A-2C present expression of SIRT6 mRNA in livers of mice injected with lipid nanoparticle-formulated SIRT6 mRNA or PBS vehicle control. (A) SIRT6 mRNA expression in mice liver 6 hours after injection of 0.1 mg/kg, 0.25 mg/kg, or 0.5 mg/kg SIRT6 mRNA formulated in MC3 LNPs compared to control. Results are presented as fold of Control mice. (B) Expression of SIRT6 protein 3 and 6 hours after injection of a 0.5 mg/kg dose of SIRT6 mRNA in MC3 formulation, as compared with Control mice injected with PBS vehicle. Also shown are expression levels of the housekeeping protein Actin. (C) Insulin-like growth factor binding protein 1 (IGFBP1) and FASN expression in mice liver 3 hours after injection of 0.5 mg/kg SIRT6 mRNA in MC3 formulation compared to mice treated with the Control vehicle. Results are presented as fold of Control Vehicle group. Average±SD of 3 mice.

FIGS. 3A-3E present the pathology of STZ-primed high fat diet (STZ-HFD) mice (NASH mice) and control mice. (A) Expression levels of inflammation markers Monocyte chemoattractant protein-1 (MCP-1), and Transforming growth factor beta (TGFβ), and immune cell infiltrate markers CD8 and F4/80 in Streptozotocin and high-fat diet-treated (STZ-HFD) mice (n=5) compared to normal (control) mice (n=3). Results, normalized to CycloA gene, are Average±SD fold to control mRNA levels as examined by RT-qPCR. (B) Levels of serum liver enzymes alkaline phosphatase (Alk Phos), alanine aminotransferase/aspartate aminotransferase (AST/ALT) (measured in International Units/Liter, IU/L). (C) Glucose levels (mg/dL) in the serum after 16 hours of starvation in normal and STZ-HFD mice. (D). Hematoxylin and eosin (H&E) staining of liver tissue sections from control mice and (E) STZ-HFD mice (40× magnification). Arrows point at exemplary hepatocytes swelling.

FIGS. 4A-4H show that treatment of STZ-HFD mice with LNP-formulated SIRT6 mRNA resulted in readily observable reductions in fat accumulation and improvements in gross histology. (A-C) Representative liver photographs from vehicle-treated STZ-HFD control mice. (D-F) Representative liver photographs from SIRT6 mRNA-treated STZ-HFD mice. (G) Liver weight (grams) of STZ-HFD mice at week 9 treated with the control vehicle or with SIRT6 mRNA. (H) Liver to Body weight ratio of STZ-HFD mice at week 9 treated with the control vehicle or with SIRT6 mRNA. Results are shown as Average±SD (Control n=10, SIRT6 mRNA n=3).

FIGS. 5A-5B are graphs showing that treatment of SZT-HFD mice with LNP-formulated SIRT6 mRNA resulted in marked reductions in serum ALT and AST levels. A) Serum AST levels (IU/L) in normal mice (mice that were not subjected to SZT-HFD), vehicle-treated SZT-HFD mice, and SIRT6 mRNA-treated SZT-HFD mice. (B) Serum ALT levels (IU/L) in normal mice, vehicle-treated SZT-HFD mice, and SIRT6 mRNA-treated SZT-HFD mice. Results are shown as Average±SD of IU/L (Control n=9, SIRT6 mRNA n=3).

FIGS. 6A-6F are photomicrographs showing H&E staining of liver sections. (A-B) Normal mice at week 9. (C-D) Control SZT-HFD mice treated with vehicle alone. (E-F) SZT-HFD mice treated with LNP-formulated SIRT6 mRNA. A/C/E and B/D/F are ×10 and ×40 magnifications, respectively.

FIGS. 7A-7C are photomicrographs showing OilRed staining of liver sections. (A) Normal mice at week 9. (B) Control SZT-HFD mice (NASH model) treated with vehicle alone. (C) SZT-HFD mice (NASH model) treated with LNP-formulated SIRT6 mRNA. 4× magnification.

FIG. 8 is a graph showing reduced expression of liver inflammation markers in SZT-HFD mice treated with SIRT6 mRNA. Expression levels of MCP-1, TNF-α, IL-6 and IL-11 in Normal (control) mice, at week 9 of age (n=3), vehicle-treated STZ-HFD (NASH) control mice (NASH model Control Vehicle) (n=10) and STZ-HFD mice treated with LNP-formulated SIRT6 mRNA (NASH model SIRT6 mRNA) (n=3). Results, normalized to CycloA gene, are Average±SD of relative mRNA levels examined by RT-qPCR.

FIG. 9 is a graph showing attenuation of liver fibrosis in SZT-HFD mice treated with SIRT6 mRNA. Expression levels of α-SMA and TIMP-1 in Normal mice, age week 9 (n=3), STZ-HFD mice (n=10) treated with the control vehicle alone, and STZ-HFD mice treated with SIRT6 mRNA (n=3). Results, normalized to CYNA gene, are Average±SD of relative mRNA levels examined by RT-qPCR.

FIG. 10 is a graph showing serum creatinine levels (mg/dl) in Normal mice, age week 9 (n=3), STZ-HFD mice (n=9) treated with the control vehicle alone, and STZ-HFD mice treated with SIRT6 mRNA (n=3).

FIGS. 11A-11C show H&E staining of SZT-HFD mouse livers at week 11 of age (n=3; FIGS. 11A-C correspond to mice #1, #2 and #3 respectively).

FIG. 12 is a graph showing the survival rate over time (mice age in weeks) of Control vehicle group (N=7) compared to SIRT6 mRNA treated SZT-HFD mice (N=6).

FIGS. 13A-13F Physical appearance of control and SIRT6 mRNA-treated mice: (A)-(C) were taken from Control vehicle group, at 16, 23 and 27 weeks of age, respectively, (D)-(F) represent the SIRT6 mRNA treated group, at 16, 23 and 28 weeks of age, respectively.

FIGS. 14A-14E present expression levels of markers of pathology of mice fed by MC-Reduced HFD for seven weeks. (A) Expression levels of MCP-1, TNF alpha (TNF-a), F4/80 and CD8a in MC-Reduced HFD mice (n=5) compared to normal (control) mice (n=3). Results, normalized to geometric (Geo) mean of Importin 8 (IPO8) & Ribosomal Protein Lateral Stalk Subunit PO (RplPO) housekeeping genes, are Average±SD fold to mRNA levels in normal mice (control) as examined by RT-qPCR. (B) Expression levels of Tissue inhibitors of metalloproteinases (TIMP-1), Collagen Type I, TR7 and TGF-β in MC-Reduced HFD mice (n=5) compared to normal (control) mice (n=3). Results, normalized to Geo mean of IPO8 & RplPO genes, are Average±SD fold to mRNA levels in normal mice (control) as examined by RT-qPCR. (C) Sirius Red staining (Arrows point at Collagen filaments) (Magnification ×10). (D) Levels of serum Triglycerides (mg/dl), AST, and ALT (measured in IU/L). (E) H&E staining of liver tissue sections (Magnification ×10).

FIGS. 15A-15B are graphs showing reduced liver toxicity in MC-Reduced-HFD mice treated with SIRT6 mRNA (A) Expression levels of MCP-1 and (B) TNFa, at treatment initiation which is referred to as the background (7 weeks under diet) (n=5) compared to Control vehicle (N=8) and SIRT6 mRNA treated mice (N=8) at week 12 under diet. Results, two weeks after the last injection, normalized to Geo mean of IPO8 & RplPO genes, are Average±SD fold to control mRNA levels as examined by RT-qPCR. T test-values are presented.

FIGS. 16A-16B are graphs showing reduced immune cells infiltration to MC-Reduced-HFD mice liver treated with SIRT6 mRNA (A) Expression levels of F4/80 and (B) CD8a markers at treatment initiation, referred to as background (7 weeks under diet) (n=5) compared to Control vehicle (N=8) and SIRT6 mRNA treated mice (N=8) at week 12 under diet. Results, from two weeks after the last injection, normalized to Geo mean of IPO8 & RplPO genes, are Average±SD fold to control mRNA levels as examined by RT-qPCR. T test-values are presented.

FIGS. 17A-17D are graphs showing reduced fibrosis in the liver of MC-Reduced-HFD mice treated with SIRT6 mRNA (A) Expression levels of TIMP-1 (B) Collagen Type I (C) TR7 and (D) TGFβ markers, at treatment initiation referred to as background (7 weeks under diet) (n=5) compared to Control vehicle (N=8) and SIRT6 mRNA treated mice (N=8) at week 12 under diet. Results, from two weeks after the last injection, normalized to Geo mean of IPO8 & RplPO genes, are Average±SD fold to control mRNA levels as examined by RT-qPCR. T test-values are presented.

FIG. 18 is a graph showing serum creatinine levels (mg/dl) in Normal mice, 12 weeks of age (n=2), MC-Reduced-HFD mice at week 7 under diet, referred to as background (the time point of treatment initiation) (N=4) or at week 12 under diet treated with the control vehicle alone (n=4) or SIRT6 mRNA (mSIRT6 ) (n=4). The results were obtained two weeks after the last treatment.

FIGS. 19A-19E present expression of different human SIRT6 mRNA in human Huh7 cells. (A-C) Protein levels of human SIRT6 expressed in Huh7 cells, endogenous human SIRT6, and mouse SIRT6 24 hours after cells transfection with increasing SiRT6 mRNA concentrations (A) 0.065 μg/ml mRNA concentration (B) 0.125 μg/ml mRNA concentration (C) 0.25 μg/ml mRNA concentration. Histone3 protein is used as the normalizing protein. The numbers #11, #48, #49, #45 and #11* (SEQ ID NO:11 with a mouse 3′UTR) represent different SIRT6 mRNA sequences with reference to corresponding SEQ ID Nos. (D) Relative protein expression of human SIRT6 mRNA sequences. Results are presented as fold of Control untreated (without transfection) cells. The graph shows three doses of transfected SIRT6 mRNA, and negative control of Lipofectamine MessengerMAX™ reagent only (Lipo Only) and control untreated cells. Results, using the human alpha globin 3′UTR target-specific primers normalized to CyCloA gene, are Average±SD fold to control mRNA levels as examined by RT-qPCR using primers. (E) Relative expression of #11 and #11* mRNAs expressed as fold of Control untreated (untransfected) cells using the human SIRT6 ORF target-specific primers normalized to CyCloA gene. Results are Average±SD fold to control mRNA levels as examined by RT-qPCR using primers.

FIGS. 20A-20F (A)-(C) Expression levels of human SIRT6 expressed in mouse primary hepatocytes using different human SIRT6 mRNA transcripts (SEQ ID Nos: #11, #48, #49 and #45). (A) Human SIRT6 protein levels 24 hours after cell transfection with 0.125 μg/mL SIRT6 mRNA, with Histone3 protein used as a normalizing protein. (B) Human SIRT6 mRNA levels 24 and 48 hours after transfection with one of three increasing concentrations. Results, using the human alpha globin 3′UTR target-specific primers normalized to CyCloA gene, are Average±SD fold to control mRNA levels as examined by RT-qPCR. (C) Protein levels of human SIRT6 expressed in mouse primary hepatocytes cells, 24 and 48 hours after cells transfection with 0.0625 μg/mL SIRT6 mRNA. Histone3 protein is used as normalizing protein. (D)-(F) representative results of human SIRT6 expression (mRNA and protein) after injection of 0.2 mg/kg SiRT6 mRNA (SEQ ID NO: 11 (#11) or SEQ ID NO: 48 (#48)) in MC3-DMA based formulation after different time points. (D) Protein levels detected at specified time points of MC3-DMA formulated SIRT6 mRNAs at specified time points. β-actin was used as normalizing protein, mouse SIRT6 levels are shown. (E) Relative human SIRT6 mRNA levels in mice liver after injection of 0.2 mg/kg SIRT6 mRNA (SEQ ID NO: 11 or SEQ ID NO: 48) in MC3-DMA based formulation (N=3 for each group). Results, using the human alpha globin 3′UTR target-specific primers normalized to CyCloA gene, are Average±SD fold to control mRNA levels as examined by RT-qPCR. (F) is a graph showing the levels of IGFBPI as fold of the control vehicle group in the liver of mice injected with human SIRT6 mRNA SEQ ID NO:11 or SEQ ID NO:48 at different time points following the injection.

FIGS. 21A-21B present the effect of different 3′UTR sequences on human SIRT6 mRNA stability and translation in mouse primary hepatocytes cells. (A) Protein levels of human and mouse SIRT6 expressed in mouse primary hepatocytes cells, 24 and 48 hours after cells transfection with 0.0625 μg/mL human SIRT6 mRNA SEQ ID NO:11 with human alpha globin 3′UTR (#11), human SIRT6 mRNA SEQ ID NO:11 with mouse alpha globin 3′UTR (#11*), and human SiRT6 mRNA SEQ ID NO:48. Histone3 protein was used as normalizing protein (B) human SIRT6 mRNA levels 24 and 48 hours after transfection with three increasing concentrations of human SIRT6 mRNA SEQ ID NO:11 with human alpha globin 3′UTR (#11) and human SiRT6 mRNA SEQ ID NO:11 with mouse alpha globin 3′UTR (#11*). Results, using the human SIRT6 ORF target-specific primers normalized to CyCloA gene, are Average±SD fold to control mRNA levels as examined by RT-qPCR.

FIG. 22A-22H is a table showing incidence of each of the four nucleotides (A, T, G, C) in their position respective to the start codon for various genes. (A) as analyzed for 799 Elevated genes in the liver (B) as analyzed for the top 503 genes of elevated expression in non-liver cells; (C) as analyzed for the top 30 genes of enriched expression in the liver; (D) as analyzed for the top 27 genes expressed only in the liver; (E) as analyzed for the top 10 genes sorted by total expression in the liver; (F) as analyzed for the top 30 genes sorted by total expression in the liver; (G) as analyzed for the top 60 genes sorted by total expression in the liver; and (H) as analyzed for the top 766 genes sorted by total expression in the liver.

FIGS. 23A-23D present the effect of different 5′UTR sequences on human SIRT6 mRNA stability and translation in human Huh7 cells. (A-B) Protein levels of human SIRT6 expressed in human HUh7 cells, 24 and 48 hours after cells transfection with 0.0625 μg/mL (A) and 0.125 μg/mL SIRT6 mRNA (B). Actin protein was used as the normalizing protein. (C) human SIRT6 mRNA levels 24 and 48 hours after transfection with three increasing concentrations. Results, using the human alpha globin 3′UTR target-specific primers normalized to CyCloA gene, are Average±SD fold to control mRNA levels as examined by RT-qPCR. (D) Representative results of human SiRT6 protein expression after injection of 0.05 and 0.25 mg/kg doses in MC3-DMA based formulation 6 hours after injection. Histone3 protein used as normalizing protein.

### DETAILED DESCRIPTION OF EMBODIMENTS

The present invention is based on the unexpected discovery that SIRT6 levels in cells can be augmented by in vivo administration of SIRT6 -encoding mRNA to treat diseases, including NASH.

The present invention therefore provides novel SIRT6 mRNA molecules for use as a medicament for affecting multiple biological processes. Accordingly, the administration of SIRT6 mRNA may affect concomitantly several pathological conditions.

For example, the increase in SIRT6 levels can be applied for treating fibrosis, as well as pre-fibrotic conditions (e.g., inflammation, endothelial or epithelial to mesenchymal transition) and post-fibrotic conditions (e.g., parenchymal injury, scarring, cirrhosis, or cancer). For example, administration of SIRT6 mRNA can halt or prevent the development of numerous organ-specific fibrotic disorders including radiation-induced fibrosis, cardiac fibrosis, pulmonary fibrosis, liver fibrosis, skin fibrosis, fibrotic conditions in the eye, brain fibrosis, kidney fibrosis and their respective stage, neurological disorders.

In addition, by affecting lipid and carbohydrate (e.g. glucose) metabolism, such as, but not limited to, affecting response to insulin, reducing the level of free fatty acids (FFA), reducing oxidative stress and affecting DNA repair, the increase in SIRT6 levels can halt or prevent the development of heart disease, stroke, type 2 diabetes, increased blood pressure, high blood sugar, excess body fat and abnormal cholesterol or triglyceride levels and can therefore be used for treating metabolic disorders (e.g., metabolic syndrome), frailty and for affecting longevity.

In particular, the SIRT6 mRNA molecules of the invention are useful in treating various liver diseases such as NAFLD, including NASH.

The invention thus provides a novel, isolated, non-native nucleic acid molecule encoding SIRT6, wherein said nucleic acid molecule comprises an mRNA molecule encoding SIRT6, a 3′ untranslated region (3′UTR), a 5′ untranslated region (5′UTR) and a poly Adenine (PolyA) chain.

The isolated nucleic acid of the invention is also referred to herein as the SIRT6 mRNA molecule of the invention. The SIRT6 mRNA molecule of the invention is non-native in that it differs from the native, wild-type SIRT6 mRNA molecule by having an alternative 3′ and/or 5′ UTR sequence, and/or by having a codon optimized or otherwise modified mRNA sequence encoding SIRT6.

The term “SIRT6 ” refers to the sirtuin 6 protein, more specifically to the mRNA encoding the sirtuin 6 protein.

In an embodiment, the SIRT6 mRNA is human SIRT6 mRNA.

According to the invention, a nucleic acid molecule or a nucleic acid sequence refers to a nucleic acid which is preferably ribonucleic acid (RNA), e.g., messenger RNA (mRNA) which may be recombinantly prepared or chemically synthesized. mRNA refers to a transcript which is produced using DNA as template and which itself codes for a peptide or protein. Accordingly, mRNA may be prepared from a DNA template by in vitro transcription.

The nucleic acid of the invention is preferably isolated. As used herein the term “isolated” when referring to the nucleic acid molecule means that the nucleic acid has been either amplified in vitro, recombinantly produced by cloning, chemically synthesized, or purified. The term is used to distinguish the nucleic acid molecule of the invention from nucleic acids found in nature.

As used herein the term “3′ untranslated region (3′UTR)” refers to a region which is the section of mRNA that immediately follows the translation termination codon and is located at the end of the nucleic acid which has a free hydroxy group. This region is transcribed but is not translated. As used herein the term “5′ untranslated region (5′UTR)” refers to a region of the mRNA that is directly upstream to the initiation codon. This region is transcribed but is not translated.

In one embodiment, the SIRT6 mRNA molecule is a linear RNA comprising RNA encoding the SIRT6 protein (also referred to herein as the human SIRT6 open reading frame (ORF)), a 5′ untranslated region (UTR), a 3′UTR, and optionally a 5′ cap and a poly-A tail. The sequence of the SIRT6 mRNA can be found in nucleotide databases (see for example, the NCBI nucleotide database). The invention encompasses variants of the SIRT6 mRNA sequence as may be found in such nucleotide databases. In a non-limiting embodiment, the native human SIRT6 open reading frame (ORF) which is transcribed to mRNA has the following sequence identified as SEQ ID NO: 11, and is also referred to herein as the wild-type or native SIRT6 mRNA:

ATGTCGGTGAATTACGCGGCGGGGCTGTCGCCGTACGCGGACAAG
GGCAAGTGCGGCCTCCCGGAGATCTTCGACCCCCCGGAGGAGCTG
GAGCGGAAGGTGTGGGAACTGGCGAGGCTGGTCTGGCAGTCTTCC
AGTGTGGTGTTCCACACGGGTGCCGGCATCAGCACTGCCTCTGGC
ATCCCCGACTTCAGGGGTCCCCACGGAGTCTGGACCATGGAGGAG
CGAGGTCTGGCCCCCAAGTTCGACACCACCTTTGAGAGCGCGCGG
CCCACGCAGACCCACATGGCGCTGGTGCAGCTGGAGCGCGTGGGC
CTCCTCCGCTTCCTGGTCAGCCAGAACGTGGACGGGCTCCATGTG
CGCTCAGGCTTCCCCAGGGACAAACTGGCAGAGCTCCACGGGAAC
ATGTTTGTGGAAGAATGTGCCAAGTGTAAGACGCAGTACGTCCGA
GACACAGTCGTGGGCACCATGGGCCTGAAGGCCACGGGCCGGCTC
TGCACCGTGGCTAAGGCAAGGGGGCTGCGAGCCTGCAGGGGAGAG
CTGAGGGACACCATCCTAGACTGGGAGGACTCCCTGCCCGACCGG
GACCTGGCACTCGCCGATGAGGCCAGCAGGAACGCCGACCTGTCC
ATCACGCTGGGTACATCGCTGCAGATCCGGCCCAGCGGGAACCTG
CCGCTGGCTACCAAGCGCCGGGGAGGCCGCCTGGTCATCGTCAAC
CTGCAGCCCACCAAGCACGACCGCCATGCTGACCTCCGCATCCAT
GGCTACGTTGACGAGGTCATGACCCGGCTCATGAAGCACCTGGGG
CTGGAGATCCCCGCCTGGGACGGCCCCCGTGTGCTGGAGAGGGCG
CTGCCACCCCTGCCCCGCCCGCCCACCCCCAAGCTGGAGCCCAAG
GAGGAATCTCCCACCCGGATCAACGGCTCTATCCCCGCCGGCCCC
AAGCAGGAGCCCTGCGCCCAGCACAACGGCTCAGAGCCCGCCAGC
CCCAAACGGGAGCGGCCCACCAGCCCTGCCCCCCACAGACCCCCC
AAAAGGGTGAAGGCCAAGGCGGTCCCCAGCTGA

It should be noted that once transcribed to mRNA the thymine (T) bases in the sequence are replaced with uridines (U), and thus the mRNA transcribed from SEQ ID NO: 11 has the same sequence albeit with uridine bases instead of the thymine bases.

As used herein when referring to the sequence of the mRNA molecule, the sequence is the transcribed version containing uridine bases instead of the thymine bases.

In one embodiment, the SIRT6 mRNA molecule comprises a coding region of 3′-5′-linked circular RNA (circRNA).

Certain modifications may be introduced to the native sequence to expand its functionality, to reduce immunity and increase stability of the mRNA molecule. Non-limiting examples of such modifications are shown in Example 6.

Therefore, the present invention also encompasses SIRT6 mRNA molecules having at least 75%, at least 80%, at least 85%, or at least 90% sequence homology to the native SIRT6 mRNA molecule of the invention. In an embodiment, the present invention refers to SIRT6 mRNA molecules having at least 75%, at least 80%, at least 85%, or at least 90% sequence homology to mRNA transcribed from the human SIRT6 ORF identified as SEQ ID No 11.

The term “% sequence homology” refers to a percentage of nucleotides which are identical in an optimal alignment between two sequences to be compared. The percent homology may be calculated using known programs, e.g., BLAST and the like.

In certain embodiments, the SIRT6 mRNA molecules are genetic versions that are more common in centenarians, i.e., genetic versions which have certain polymorphisms that are particularly associated with longevity.

The present invention also encompasses variants of the SIRT6 mRNA molecules. The variants may include nucleic acid substitutions which do not reduce the functionality of the SIRT6 mRNA, e.g., its ability to transcribe a functional SIRT6 protein.

By the term “variant” it is meant a SIRT6 mRNA molecule in which one or more nucleotides are deleted, substituted, or added, wherein these alterations do not abolish the functionality of the SIRT6 mRNA herein described.

It should be appreciated that by the term “added”, as used herein it is meant any addition of one or more nucleic acids to the sequences described herein.

It should be appreciated that by the term “substituted”, as used herein it is meant any substitution of one or more nucleic acids of the sequences described herein.

It should be appreciated that by the term “deleted”, as used herein it is meant any deletion of one or more nucleic acids from the sequences described herein.

To increase stability the mRNA may be modified and capped by adding for example a CAP 1 structure at the 5′ end of the mRNA. The capping may be performed using methods known in the art for example using, but not limited to, CleanCap1, CleanCap AG, or CleanCap AG (3′OMe) reagents, e.g., as described in the Examples below.

Accordingly, the present invention also encompasses various modifications of the SIRT6 mRNA which may increase the molecule's stability. Non-limiting examples of chemical modifications include 6-methyladenosine (m6A), N6,2′-O-dimethyladenosine (m6Am), 8-oxo-7,8-dihydroguanosine (8-oxoG), pseudouridine (Ψ), 5-methylcytidine (m5C), and N4-acetylcytidine (ac4C), sugar modifications, e.g., 2′ O-methyl oligoribonucleotides (O-Me), 2′ O-methoxyethyl-RNA (MOE), 2′-F alterations, locked nucleic acids (LNA), and combinations thereof, and backbone modifications e.g., phosphorothioate (PS) linkage, Morpholino oligonucleotides, and phosphonoacetate oligonucleotides (PACE), 2′-amino-2′-deoxynucleotide. 2′-azido-2′-deoxynucleotide, 2′-fluoro-2′-deoxynucleotide, 2′.O)-methyl-nucleotides, the super dressing agent (2′ of 2′ sugar super modifier), heat endurance reinforcing agent, 2′-fluoro-2′-deoxyadenosine-the 5-triphosphoric acid, 2′ of 2′-modification-Fluoro-2′-deoxycytidine-5′-triphosphoric acid, 2′-fluoro-2′-deoxyguanosine-5′-triphosphoric acid, 2′-fluoro-2′-BrdU-5′-tri-Phosphoric acid, 2′-O-methyladenosine-5′-triphosphoric acid, 2′-O-methylcytidine-5′-triphosphoric acid, 2′-O-methylguanosine-5′-triphosphoric acid, 2′-O-methyluridine-5′-triphosphoric acid, pseudouridine-5′-triphosphoric acid, 2′-O-methylinosine-5′-triphosphoric acid, 2′-amino-2′-Deoxycytidine-5′-uriphosphoric acid. 2′-amino-2′-BrdU-5′-triphosphoric acid, 2′-azido-2′-deoxycytidine-5′-tri-Phosphoric acid, 2′-azido-2′-BrdU-5′-triphosphoric acid, 2′-O-methyl pseudouridine-5′-triphosphoric acid, 2′-O-methyl-5′-first Base UTP, 2′-azido-2′-deoxyadenosine-5′-triphosphoric acid, 2′-amino-2′-deoxyadenosine-5′-tri-phosphorus Acid, 2′-fluoro-thymidine. 5′-triphosphoric acid, 2′-azido-2′-deoxyguanosine-5′-triphosphoric acid, 2′-amino-2′-deoxyguanosine-5′-triphosphoric acid and N4-methylcytidine-5′-triphosphoric acid.

In a specific embodiment, the uridines of the mRNA molecule are substituted with the chemically modified N1-Methylpseudouridine-5′-Triphosphate.

The SIRT6 mRNA may also be modified by codon usage optimization. As used herein the term “codon usage optimization” refers to the replacement of the codon usage of the host without modifying the amino acid sequence of the encoded protein. Codon optimization can be used to increase the stability of the molecule, to reduce immunogenicity and to increase protein expression.

Protein sequences can be encoded by an enormous multitude of possible nucleotide sequences. The degenerate mapping between amino acids and synonymous codons entails an exponential relationship between the number of potential nucleotide sequences and the length of the polypeptide chain. However, different nucleotide sequences encoding the same protein may exhibit dramatically different outcomes in expression systems.

Codon optimization is a procedure designed to increase gene expression based on a scoring function. The scoring functions include optimization of the fraction of G and C bases, matching the codon usage bias of the host expression system and/or attempting to disrupt the formation of mRNA secondary structure. The expression levels of proteins are highly correlated with codon usage bias. The frequency of codons in a DNA sequence is positively correlated with the corresponding tRNA in a species, and the tRNA concentration determines the number of amino acids available for protein translation extension, which in turn affects the efficiency of protein synthesis. Thus, rare codons tend to reduce the rate of translation and even cause translation errors.

The present invention thus provides codon optimized SIRT6 mRNA molecules. These molecules were constructed based on the native wild-type sequence (SEQ ID NO: 11) and are presented as SEQ ID Nos: 41-49 as detailed in Example 6.

It should be noted that once transcribed to mRNA the thymine (T) bases in the sequences are replaced with uridines (U), and thus the mRNA transcribed from SEQ ID NOs: 41-49 have the same sequence albeit with uridine bases instead of the thymine bases. As used herein when referring to the sequence of the mRNA molecule, i.e., SEQ ID NO: 11, or SEQ ID Nos: 41-49 the sequence is the transcribed version containing uridine bases instead of the thymine bases. As shown in the examples, the codon optimized SIRT6 mRNA transcribed from SEQ ID NO: 48 was highly efficient in inducing protein translation.

In one embodiment, the present invention therefore provides an mRNA molecule transcribed from SEQ ID NO:48.

Ubiquitination is the biochemical process in which proteins are marked by ubiquitin and thereby targeted for degradation. One way of increasing stability and half-life of SIRT6 in cells is by modifying the molecule to be less prone to ubiquitination and degradation. Accordingly, the SIRT6 mRNA may also be modified by replacing amino acid residues that are prone to be phosphorylated and thereby lead to ubiquitination of SIRT6. For example, SIRT6 is known to be phosphorylated at Ser338 by the kinase AKT1, which induces the interaction and ubiquitination of SIRT6 by MDM2, targeting SIRT6 for protease-dependent degradation (Umadevi Thirumurthi 2014).

Therefore, in one embodiment, the SIRT6 mRNA may be modified to prevent SIRT6 phosphorylation by mutating the Ser338 residue.

It is also known that SIRT6 is a substrate of anaphase-promoting complex/cyclosome (APC/C). APC/C, together with its co-activators CDC20 and CDH1, mediate SIRT6 degradation via the ubiquitination-proteasome pathway (Wang et al 2021). The APC mediates degradation in a D-Box-Dependent manner.

Therefore, in one embodiment, the SIRT6 mRNA may be modified to prevent or reduce SIRT6 degradation by substituting the arginine and/or leucine amino acid residues within the RxxL motif of the first D-box-activating domain of the SIRT6 protein with alanine (e.g., a substitution from RVGL to AVGA at positions 103-106).

The ubiquitin-specific peptidase USP10, a tumor suppressor that often has low expression in human cancers, is a SIRT6 -specific de-ubiquitinase. Suppression of USP10 expression promotes human colon cancer cell growth and tumor formation through proteasomal degradation of SIRT6 (Kong et al., 2013)

Therefore, in one embodiment, the SIRT6 mRNA may be modified in the C-terminal site, between positions 263-334, to prevent SIRT6 ubiquitination.

The ubiquitin ligase CHIP (carboxyl terminus of Hsp70-interacting protein) ubiquitinates SIRT6 at K170, which stabilizes SIRT6 and prevents SIRT6 canonical ubiquitination by other ubiquitin ligases. SIRT6 K170 mutation (mutated the lysine at position 170 in wildtype SIRT6 to arginine K170R SIRT6 ) increases SIRT6 half-life and prevents proteasome-mediated degradation (Sarah M. Ronnebaum et al., 2013).

Therefore, in one embodiment, the SIRT6 mRNA may be modified at position K170 to arginine, namely K170R, to increase SIRT6 half-life.

The linear SIRT6 mRNA molecules of the invention may also be modified to remove or alter naturally occurring miRNA binding sites.

miRNAs are small, single-stranded, noncoding RNAs, containing approximately 21 nucleotides (nt) that regulate gene expression at a posttranscriptional level. They complementarily bind to the 3′ untranslated region (3′ UTR) of their target mRNAs causing their degradation, translational repression, and/or deadenylation (Winter J et al, 2009).

The expression of SIRT6, has been shown to be directly and/or reciprocally regulated by different miRNA identified by their seed target to human SIRT6 3′UTR (a list of miRNA sequences can be found for example in http://mirdb.org/data base). These include but are not limited to hsa-miR-766-3p, hsa-miR-33b-5p, hsa-miR-33a-5p, hsa-miR-33b, hsa-miR-10396b-5p, hsa-miR-6787-5p, hsa-miR-137-3p, hsa-miR-1908-5p, hsa-miR-663a, hsa-miR-541-3p, hsa-miR-654-5p and/or other miRNA with only partial match to miRNA seed target including, but not limited to hsa-miR-122-5p.

Therefore, to effectively regulate SIRT6 expression in the diseased tissue, microRNA binding sites can be removed from the native SIRT6 3′UTR sequence, to increase protein expression in the tissue. Alternatively, one or more point-mutations or nucleic acid substitutions may be introduced to the 3′ UTRs of the mRNA molecule to alter (i.e., disable) these microRNA binding sites or the whole sequence of the 3′UTR may be replaced with a sequence that does not contain a miRNA targeting site.

For example, an inverse correlation between SIRT6 levels and miR-122 or miR-33 has been observed in hepatocytes where miR-122 (partial seed match) and miR-33 (full seed match) target the 3′UTR regions of SIRT6 mRNAs, block its translational process and lead to gene silencing.

Therefore, in specific embodiments one or more of hsa-miR-122-5p (e.g. positions 122-146, 197-218, and 228-249 of the SIRT6 3′UTR), hsa-miR-33a-5p (e.g. position 479-486 of the SIRT6 3′UTR), and hsa-miR-33b binding sites may be removed from the 3′UTR of SIRT6 to improve SIRT6 protein expression at different stages of liver fibrosis. The following is the sequence of the native SIRT6 gene 3′UTR, denoted as SEQ ID NO: 57:

CCAGGGTGCTTGGGGAGGGTGGGGCTTTTTGTAGAAACTGTGGAT
TCTTTTTCTCTCGTGGTCTCACTTTGTTACTTGTTTCTGTCCCCG
GGAGCCTCAGGGCTCTGAGAGCTGTGCTCCAGGCCAGGGGTTACA
CCTGCCCTCCGTGGTCCCTCCCTGGGCTCCAGGGGCCTCTGGTGC
GGTTCCGGGAAGAAGCCACACCCCAGAGGTGACAGGTGAGCCCCT
GCCACACCCCAGCCTCTGACTTGCTGTGTTGTCCAGAGGTGAGGC
TGGGCCCTCCCTGGTCTCCAGCTTAAACAGGAGTGAACTCCCTCT
GTCCCCAGGGCCTCCCTTCTGGGCCCCCTACAGCCCACCCTACCC
CTCCTCCATGGGCCCTGCAGGAGGGGAGACCCACCTTGAAGTGGG
GGATCAGTAGAGGCTTGCACTGCCTTTGGGGCTGGAGGGAGACGT
GGGTCCACCAGGCTTCTGGAAAAGTCCTCAATGCAATAAAAACAA
TTTCTTTCTTGCA

Regulation of expression in multiple tissues can be accomplished through removal of one or several microRNA bindings sites.

The 3′ UTR of mRNA plays an important role in post-transcriptional control of gene expression, such as stability, translation, and localization (Mayr C, 2019). Much of its regulatory function is mediated through embedded sequence and structure motifs, such as microRNA target sites and various AU-rich and GU-rich elements for stability and/or translational controls.

In one embodiment the 3′UTR region is replaced to increase the stability and translation of the SIRT6 molecule (e.g., with alpha globin 3′UTR, preferably human alpha globin 3′UTR). Therefore, in one embodiment, the present invention provides a native SIRT6 mRNA molecule with a human alpha globin 3′UTR, e.g., a molecule transcribed from SEQ ID NO: 11 and human alpha globin 3′UTR as denoted in SEQ ID NO: 52, or a sequence having at least 95%, or at least 99% sequence homology with SEQ ID NO: 52.

In another embodiment, the present invention provides a codon optimized SIRT6 molecules with a human alpha globin 3′UTR, e.g., a molecule transcribed from any one of SEQ ID NOs: 41-49 and alpha globin 3′UTR (e.g., SEQ ID NO: 52, or a sequence having at least 95%, or at least 99% sequence homology with SEQ ID NO: 52). In one specific embodiment the present invention provides a molecule transcribed from SEQ ID NO: 48 and human alpha globin 3′UTR (SEQ ID NO: 52, or a sequence having at least 95%, or at least 99% sequence homology with SEQ ID NO: 52).

The 5′-untranslated region (5′-UTR) lies within the noncoding sequence upstream of coding sequences and plays a pivotal role in regulating gene expression. Efficient translation initiation in mammalian species depends mainly on two factors: (1) the Kozak consensus (also termed the Kozak sequence) which refers to the nucleic acid motif that functions as the protein translation initiation site, and (2) the secondary structure that may embed the Kozak consensus to obscure the essential translation initiation signals. These factors contribute to the proper positioning of ribosomes at the start codon to allow efficient transition from translation initiation to elongation. Transcribed 5′-UTRs are composed of a variety of RNA-based regulatory elements including the 5′-cap structure, secondary structures, RNA-binding protein motifs, upstream open-reading frames (uORFs), internal ribosome entry sites, terminal oligo pyrimidine (TOP) tracts, and G-quadruplexes. These elements can alter the efficiency of mRNA translation; some can also affect mRNA transcript levels via changes in stability or degradation.

The 5′ UTR may be replaced with 5′ UTR of known genes, e.g., a 5′UTR used in expression vectors for expression of viral genes, or 5′UTR sequences that are used in mRNA vaccines (e.g., the COVID vaccine).

In accordance with the present invention, the 5′ UTR may be replaced with a consensus sequence prepared as described in Example 8 based on the sequences of liver genes, such that it optimizes the expression of the SIRT6 mRNA in the liver. The consensus 5′UTR sequence in denoted as SEQ ID NO: 54.

Therefore, in one embodiment, the present invention provides a native SIRT6 mRNA molecule with a replaced 5′UTR, e.g., a molecule transcribed from SEQ ID NO: 11 and the consensus 5′UTR (SEQ ID NO: 54).

In another embodiment, the present invention provides a codon optimized SIRT6 mRNA molecule with the consensus 5′UTR (SEQ ID NO: 54), e.g., a molecule transcribed from any one of SEQ ID NOs: 41-49 and the consensus 5′UTR (SEQ ID NO: 54). In one specific embodiment the present invention provides a molecule transcribed from SEQ ID NO: 48 and the consensus 5′UTR (SEQ ID NO: 54). In another embodiment, the present invention provides a molecule transcribed from SEQ ID NO: 48 and a 5′UTR used in expression vectors for expression of viral genes (e.g., SEQ ID NO: 56).

In a specific embodiment, the present invention provides a molecule comprising SEQ ID NO: 48, human alpha globin 3′UTR (e.g., SEQ ID NO: 52 or a sequence having at least 95%, or at least 99% sequence homology with SEQ ID NO: 52), and the consensus 5′UTR (SEQ ID NO: 54). In another specific embodiment, the present invention provides a molecule comprising SEQ ID NO: 48, human alpha globin 3′UTR (e.g., SEQ ID NO: 52 or a sequence having at least 95%, or at least 99% sequence homology with SEQ ID NO: 52), and a 5′UTR used in expression vectors for expression of viral genes (e.g., SEQ ID NO: 56).

In an embodiment, the uridines in the molecules transcribed from SEQ ID NOs: 11 and 41-49 are substituted with the chemically modified N1-Methylpseudouridine-5′-Triphosphate.

Optionally, any one of the SIRT6 mRNA molecules of the invention further comprises at least 40, at least 80, at least 100, or at least 120 consecutive adenine nucleotides (A), also referred to as a “Poly A tail”. In an embodiment, the molecules of the invention comprise about 120 consecutive adenine nucleotides at the 3′ end of the molecule.

In another aspect, the present invention provides a method for treating a disease or disorder or for increasing longevity in a subject in need thereof comprising a step of administering mRNA encoding SIRT6 to said subject wherein said disease or disorder is at least one of a fibrotic condition, a pre-fibrotic condition, a post-fibrotic condition, frailty, a metabolic disorder, a liver disease, a kidney disease, or cancer.

It is to be understood that the terms “treat”, “treating”, “treatment” or forms thereof, as used herein, mean reducing, preventing, curing, reversing, ameliorating, attenuating, alleviating, minimizing, suppressing, slowing, or halting, in whole or part, the deleterious effects or symptoms of a disease or a condition or delaying the onset of one or more clinical indications of a disease or disorder, as defined herein.

The terms “fibrosis” and “a fibrotic condition” are used interchangeably herein and refer to pathological tissue healing in which connective tissue replaces normal parenchymal tissue, leading to considerable tissue re-modelling and the formation of permanent scar tissue. Fibrosis may occur in any one of multiple organs including, but not limited to liver, lungs, kidney, skin, eye, or the heart muscle. As used herein the term fibrosis or a fibrotic condition also refers to different stages of the tissue fibrosis.

“Cirrhosis” is a non-limiting example of a post-fibrotic condition, an advanced fibrosis. Cirrhosis, also known as liver cirrhosis or hepatic cirrhosis, is an end-stage liver disease, characterized by impaired liver function caused by the formation of a fibrotic scar tissue, due to damage caused by liver disease. Damage causes tissue repair and subsequent formation of scar tissue, which over time replaces normal functioning tissue leading to the impaired liver function of cirrhosis.

A “metabolic disorder” is a group of disorders that negatively alters the body's processing and distribution of macronutrients such as proteins, fats, and carbohydrates.

“Metabolic syndrome” is a cluster of conditions that occur together, increasing the risk of heart disease, stroke, and type 2 diabetes. These conditions include increased blood pressure, high blood sugar, excess body fat around the waist, and abnormal cholesterol or triglyceride levels.

“Liver diseases” refers to conditions that affect primarily the liver. In one specific embodiment the liver disease is a non-alcoholic fatty liver disease (NAFLD) (e.g., nonalcoholic steatohepatitis (NASH)) which is a chronic liver disease that is manifested clinically by an increase in hepatic triglycerides, inflammation (e.g., steatohepatitis), and fibrosis.

In accordance with the invention, the term “cancer” encompasses both solid tumors and hematological cancers. For example, but not limited to, adenocarcinoma, breast carcinoma, ovarian carcinoma, non-small cell lung cancer, bladder cancer, prostate cancer, colon cancer, hepatocellular carcinoma, squamous cell carcinoma or glioma, bone sarcoma, tendon sarcoma, cartilage sarcoma, muscle sarcoma, fat sarcoma, myeloma, leukemia or lymphoma.

Accordingly, in an aspect, the present invention provides methods of inhibiting proliferation, and/or inducing cell death, and/or reducing metastasis of adenocarcinoma, breast carcinoma, ovarian carcinoma, non-small cell lung cancer, bladder cancer, prostate cancer, colon cancer, hepatocellular carcinoma, squamous cell carcinoma or glioma, bone sarcoma, tendon sarcoma, cartilage sarcoma, muscle sarcoma, fat sarcoma, myeloma, leukemia or lymphoma cancer cells comprising contacting said cells with an effective amount of SIRT6 mRNA as described herein.

A “therapeutically effective amount” of the SIRT6 mRNA according to the invention, or the pharmaceutical composition according to the invention for purposes herein defined is determined by such considerations as are known in the art in order to produce a desired therapeutic and/or prophylactic effect, cure, arrest or at least alleviate or ameliorate the medical condition. For any preparation used in the methods of the invention, the dosage or the therapeutically effective amount can be estimated initially from in vitro cell culture assays or based on suitable animal models. In the context of therapeutic or prophylactic applications, the amount of the SIRT6 mRNA, or a composition comprising same, administered to the subject will vary depending on the composition, the degree, type, and severity of the disease or risk of the disease and on the characteristics of the subject.

As used herein, a “subject” or “patient” is a mammal, such as a cat, dog, or a household or farm animal. Typically, the subject is a human, such as a human suffering from conditions sensitive to the SIRT6 mRNA and pharmaceutical compositions of the present invention. The term “subject” and “patient” can be used interchangeably.

As used herein, “administering” or the “administration” of SIRT6 mRNA or pharmaceutical composition to a subject includes any route of introducing or delivering to a subject an effective amount of SIRT6 mRNA or pharmaceutical composition of the present invention to perform its intended function. Administration can be carried out by any suitable route, including but not limited to, intravenous, subcutaneous or intramuscular injection.

In some embodiments the isolated SIRT6 mRNA according to the invention or the pharmaceutical composition according to the invention is administered to the subject as a single dose or in multiple doses.

In some embodiments, the SIRT6 mRNA and the pharmaceutical compositions of the invention are administered twice weekly, once weekly (for example for one, two, three or four months), or once every two, three or four weeks (for example for six, ten or twelve months. The SIRT6 mRNA and the pharmaceutical compositions of the invention may also be administered as a chronic treatment, i.e., indefinitely.

In one embodiment, the SIRT6 mRNA and the pharmaceutical compositions of the invention are administered for a first cycle of once weekly administrations for 4 weeks and then the treatment is halted and readministered for at least one additional cycle of once weekly administrations for 4 weeks, several months afterwards. In one embodiment, the SIRT6 mRNA and the pharmaceutical compositions of the invention are administered at a dose of between about 0.01 mg/Kg and 1 mg/Kg, e.g., 0.03 mg/Kg, 0.05 mg/Kg, 0.1 mg/Kg or 0.5 mg/Kg.

As shown in the examples below, SIRT6 mRNA was administered to two different mouse models of NASH. Administration of the mRNA resulted in significant improvements in the physiological parameters (of fatty liver, inflammation and/or fibrosis) as well as the survival of the mice.

For example, SIRT6 mRNA significantly reduced fat accumulation in the liver, as could be visualized by the liver's appearance, the reduced liver weight, and the ratio between liver and body weight in the SIRT6 mRNA treated mice as compared with the non-treated controls. In addition, AST and ALT levels in the serum were reduced. The level of hepatocytes swelling, and ballooning was also reduced. A reduction in the levels of inflammatory cytokines e.g., IL-6, TNF-α, MCP-1 and IL-11 and a significant decline in infiltrating macrophages and lymphocytes were also observed. Furthermore, introduction of SIRT6 mRNA attenuated fibrosis formation as manifested by the reduction in TIMP-1 and α-SMA expression levels, Collagen Type I and TR7 gene expression.

Interestingly, also a reduction in the elevated levels of serum creatinine was observed.

Thus, in an embodiment, the present invention provides a method for treating NAFLD (e.g., nonalcoholic steatohepatitis (NASH)) comprising a step of administering mRNA encoding SIRT6 to a subject in need thereof.

In a specific embodiment, the present invention provides a method for reducing fat accumulation, fibrosis and/or inflammation in the liver by administering the SIRT6 mRNA of the invention.

In a specific embodiment, the present invention provides a method for reducing glucose, fat, and/or triglyceride levels in the serum of a subject in need thereof by administering the SIRT6 mRNA of the invention.

In an embodiment, the present invention also provides a method of treating a kidney disease, e.g., chronic kidney disease (CKD). The present invention also provides a method for lowering the levels of serum creatinine in a subject suffering from a kidney disease.

The SIRT6 mRNA molecule of the invention may be administered as part of a combination therapy together with an additional therapeutic agent.

As a non-limiting example, the SIRT6 of the invention may be administered in combination with an anti-miRNA molecule which is directed to a miRNA that negatively regulates SIRT6 expression and is induced at various stages of fibrosis. These include, but are not limited to, hsa-miR-122-5p, hsa-miR-33a, hsa-miR-34a.

The terms “SIRT6 miRNAs” and “SIRT6 -targeted miRNAs” are used interchangeably herein and refer to any miRNA that can potentially affect SIRT6 mRNA degradation and/or cause SIRT6 mRNA translational repression. The following is a non-limiting list of SIRT6 miRs:

miR-33A (GUGCAUUGUAGUUGCAUUGCA) (SEQ ID NO: 1), miR-33B (GUGCAUUGCUGUUGCAUUGC) (SEQ ID NO: 2), miR-122 (UGGAGUGUGAC AAUGGUGUUUG) (SEQ ID NO: 3), miR-370 (GCCUGCUGGGGUGGAACCUGGU) (SEQ ID NO: 4), miR-34c-5p, miR-351-5p, miR-378b, miR-186, miR-34a, miR-125b, miR-495, miR-766, miR-25, miR-338-3p, miR-92a-3p. Additional SIRT6 miRs can be identified using bioinformatic methods (non-limiting examples include miRNA target prediction database (MiRDB), and TargetScan)

The following is a non-limiting list of SIRT6 anti-miRs:

Anti miR-33A:
(SEQ ID NO: 5)
UGCAAUGCAACUACAAUGCAC
Anti miR-122:
(SEQ ID NO: 6)
CAAACACCAUUGUCACACUCCA

Anti-miR-370, Anti-miR-34c-5p, Anti-miR-351-5p, Anti-miR-378b, Anti miR-186, Anti-miR-34a, Anti-miR-125b, Anti-miR-495, Anti-miR-25, Anti-miR-338-3p, Anti-miR-92a-3p, Anti-miR-766-3p, Anti-miR-33b, Anti-miR-10396b-5p, Anti-miR-6787-5p, Anti-miR-137-3p, Anti-miR-1908-5p, Anti-miR-663a, Anti-miR-541-3p, and Anti-miR-654-5p.

As indicated above, the SIRT6 mRNA molecule of the invention may be administered as part of a combination therapy. For example, the SIRT6 mRNA molecule may be administered with conventional anti-cancer therapeutics, including but not limited to, chemotherapy (e.g., alkylating agents, plant alkaloids, anti-metabolites, anti-tumor antibiotics and corticosteroids), biological and immunotherapy (e.g., tyrpsine kinase inhibitors, immune checkpoint inhibitors, antibodies, and immune cells modulators) or irradiation.

In such cases, the SIRT6 mRNA may be directed to treat the tumor itself (namely, to induce senescence or lead to apoptotic death), it may reduce metastasis, and/or reduce resistance to conventional anti-cancer drugs.

The SIRT6 mRNA molecule of the invention may also be administered as part of a combination therapy with another agent that increases SIRT6 levels, e.g., an adeno associated virus (AAV) vector comprising the SIRT6 gene. In such case, the AAV vector may be administered once and the SIRT6 mRNA molecule of the invention may be administered as a follow up therapy once or multiple times as a chronic treatment.

Administration according to the present invention may be performed by any of the following routes: oral administration, intravenous administration, intramuscular administration, intraperitoneal administration, intrathecal administration, subcutaneous administration, intra-rectal administration, intranasal administration (e.g., by inhalation using an aspirator), ocular administration, or topical administration.

The SIRT6 mRNAs as herein defined, or any pharmaceutical compositions comprising the same may be administered to a subject prior to or post disease onset (i.e., during the disease), in a single dose or in multiple doses.

In an embodiment, the SIRT6 mRNA is conjugated to a targeting moiety which targets the SIRT6 mRNA to a specific tissue or organ.

The SIRT6 mRNA molecule may be administered in a non-encapsulated, naked form.

To function in vivo, mRNA requires safe, effective, and stable delivery systems that protect the nucleic acid from degradation and that allow cellular uptake and mRNA release. For that purpose, various carriers may be used, for example, lipid nanoparticles. For a description of potential lipid nanoparticles suitable for encapsulating the mRNA molecule of the invention see Hou et al 2021.

Accordingly, the SIRT6 mRNA of the invention, may be administered in a lipid-encapsulated form, e.g., encapsulated in lipid nanoparticles (LNP). In one embodiment, the SIRT6 mRNA molecule is formulated in the ionizable lipid D-Lin-MC3-DMA (MC3) formulation using protocols well known in the art, for example as described in the Examples below. In another embodiment the SIRT6 mRNA molecule is formulated in the ionizable amino lipid, Lipid 5, using protocols well known in the art.

In some embodiments the methods according to the invention are wherein said methods further comprise administering to a subject in need thereof an additional therapeutic agent. In specific embodiments the additional therapeutic agent may be an anti-miR, siRNA, antisense oligonucleotide (ASO), miRNA, an mRNA molecule encoding another protein, AAV encoding SIRT6 gene and/or a small molecule which increases SIRT6 enzymatic activity, and in the case of cancer therapy said additional therapeutic agent may also be a chemotherapeutic agent or an immune check point modulator.

In some embodiments, the method of the invention further comprises measuring the levels of SIRT6 mRNA or the levels of SIRT6 protein in said subject, prior to and/or during treatment, wherein the types and amounts of said SIRT6 mRNA are adjusted according to the measured levels of SIRT6. The levels of SIRT6 mRNA or SIRT6 protein may be measured using any method known in the art, for example using the methods demonstrated in the Examples below.

In some embodiments, the present invention provides pharmaceutical compositions comprising SIRT6 mRNA as disclosed herein and a suitable carrier or excipient or diluent. The pharmaceutical compositions may be used in the methods of treatment, or preventative treatment, of a disease as described herein. The “pharmaceutical composition” of the invention generally comprises an effective amount of native or modified SIRT6 mRNA as herein defined and a buffering agent, an agent which adjusts the osmolarity of the composition and optionally, one or more pharmaceutically acceptable carriers, excipients and/or diluents as known in the art.

The effective amount may be determined in relation to a mammalian subject, preferably a human subject.

As used herein the term “pharmaceutically acceptable carrier, excipient or diluent” includes any solvents, dispersion media, coatings, antibacterial and antifungal agents, and the like, as known in the art. The carrier can be solvent or dispersion medium containing, for example, water, phosphate buffer, saline, ethanol, polyol (for example, glycerol, propylene glycol, and liquid polyethylene glycol, and the like), suitable mixtures thereof, and vegetable oils. Each carrier should be both pharmaceutically and physiologically acceptable in the sense of being compatible with the other ingredients and not injurious to the subject.

In some embodiments, the SIRT6 mRNA of the invention may be conjugated to or encapsulated in delivery carriers, such as lipid nanoparticles, cells or associated with polymers or peptides. The SIRT6 mRNA of the invention may also be provided as free mRNA in solution.

In other embodiments the pharmaceutical composition according to the invention further comprises an additional therapeutic agent.

### EXAMPLES

According to the invention, standard methods may be used for preparing recombinant nucleic acids, culturing cells, and introducing nucleic acids, in particular RNA into cells, specifically lipofection, for example as described below.

### Materials and Methods

### RNA Extraction

RNA was isolated from the cells using the PureLink™ RNA Isolation Kit (Thermo fisher scientific cat-12183018A) according to the manufacturer's protocol.

### Quantitative Real-Time PCR

1 μg of total RNA was reversed transcribed with the High-capacity reversed transcription kit (Thermo scientific) according to the manufacturer's instructions. qRTPCR was performed with the PowerUp™ SYBR™ Green Master Mix, (ThermoFisher) according to the manufacturer's protocol with minor adjustments.

Primer sequences are as follow:

SIRT6 Forward primer:
SEQ ID NO: 7
CAGAGCTGCACGGAAACATG
SIRT6 Reverse primer:
SEQ ID NO: 8
TCATCAGCGAGCATCAGGTC,
beta actin Forward primer:
SEQ ID NO: 9
AGCCATGTACGTAGCCATCC,
beta actin Reverse primer:
SEQ ID NO: 10
CTCTCAGCTGTGGTGGTGAA,
CycloA Forward primer:
SEQ ID NO: 13
AATGGCACTGGTGGCAAGTC,
CycloA Reverse primer:
SEQ ID NO: 14
CAGTCTTGGCAGTGCAGATG,
IGFBP1 Forward primer:
SEQ ID NO: 15
AGCCCAGAGATGACAGAGGA,
IGFBP1 Reverse primer:
SEQ ID NO: 16
GTTGGGCTGCAGCTAATCTC,
FASN Forward primer:
SEQ ID NO: 17
TGGGTTCTAGCCAGCAGAGT,
FASN Reverse primer:
SEQ ID NO: 18
ACCACCAGAGACCGTTATGC,
MCP-1 Forward primer:
SEQ ID NO: 19
AGGTCCCTGTCATGCTTCTG,
MCP-1 Reverse primer:
SEQ ID NO: 20
TCTGGACCCATTCCTTCTTG,
CD8a Forward primer:
SEQ ID NO: 21
ACTACCAAGCCAGTGCTGCGAA,
CD8a Reverse primer:
SEQ ID NO: 22
ATCACAGGCGAAGTCCAATCCG,
TNF-a Forward primer:
SEQ ID NO: 23
CGTCAGCCGATTTGCTATCT,
TNF-a Reverse primer:
SEQ ID NO: 24
CGGACTCCGCAAAGTCTAAG,
IL-6 Forward primer:
SEQ ID NO: 25
TACCACTTCACAAGTCGGAGGC,
IL-6 Reverse primer:
SEQ ID NO: 26
CTGCAAGTGCATCATCGTTGTTC,
IL-11 Forward primer:
SEQ ID NO: 27
CTGACGGAGATCACAGTCTGGA,
IL-11 Reverse primer:
SEQ ID NO: 28
GGACATCAAGTCTACTCGAAGCC,
aSMA Forward primer:
SEQ ID NO: 29
CTGACAGAGGCACCACTGAA,
aSMA Reverse primer:
SEQ ID NO: 30
CATCTCCAGAGTCCAGCACA,
TIMP-1 Forward primer:
SEQ ID NO: 31
ATTCAAGGCTGTGGGAAATG,
TIMP-1 Reverse primer:
SEQ ID NO: 32
CTCAGAGTACGCCAGGGAAC
TR7 Forward primer:
SEQ ID NO: 33
CACCTTCCTCTGGCACAGTTAC,
TR7 Reverse primer:
SEQ ID NO: 34
TCCCTTCCTCGATGCCACTT
TGFβ Forward primer:
SEQ ID NO: 35
TTGCTTCAGCTCCACAGAGA,
TGFβ Reverse primer:
SEQ ID NO: 36
TGGTTGTAGAGGGCAAGGAC
IPO8 Forward primer:
SEQ ID NO: 37
CGTGACAGTAGATACCAACGCTC,
IPO8 Reverse primer:
SEQ ID NO: 38
CATAGCACTCGGCATCTTCTCC
RpIPO Forward primer:
SEQ ID NO: 39
GCTTCGTGTTCACCAAGGAGGA,
RpIPO Reverse primer:
SEQ ID NO: 40
GTCCTAGACCAGTGTTCTGAGC
Human a-Globin 3′UTR Forward primer:
SEQ ID NO: 50
CTCGGTGGCCTAGCTTCTT,
and
Human a-Globin 3′UTR Reverse primer:
SEQ ID NO: 51
CCGCCCACTCAGACTTTATT.

The reaction was performed with the CFX Connect Real-Time PCR Detection System (Bio-Rad) with Sybr green fluorescence and quantified with the Bio-rad CFX maestro program.

### MC3 Formulation Preparation

Dlin-MC3-DMA (MC3), cholesterol, DSPC, and PEG-DMG were mixed at a molar ratio of 50:38.5:10:1.5 with absolute ethanol in a tube. Citric acid buffer (50 mM)
was used to suspend SIRT6 mRNA. To create LNPs, a dual syringe
pump was used to transport the solution through the NanoAssembler—micromixer from Precision NanoSystem (Vancouver, British Columbia, Canada) at a total flow rate of 12 mL/min.

The particles were then transferred into dialysis overnight against PBS. Particles in PBS were analyzed for size and uniformity by dynamic light scattering (DLS). Zeta potential was determined using the Malvern-zeta-sizer (Malvern, Worchestershire, UK).
RNA encapsulation in LNPs was calculated according to
Quant-iT—RiboGreen—RNA Assay Kit (Thermo Fisher, Waltham, MA, USA), by calculating the percentage encapsulation at 100%—(RNA-LNPs/RNA-LNPs with triton).

### Western Blot Analysis

Cells were lysed in cold lysis buffer (50 mM Tris-HCl, pH 7.4, 150 mM NaCl, 1 mM DTT, 10% Glycerol, 1 mM MgCl2, 0.1% NP-40) for 10 minutes.

Lysates were sonicated and centrifuged for 10 minutes at 17000 g, at 4° C. Laemmli buffer was added to each sample and boiled for 5 minutes at 100° C.

10-100 μg of total protein was resolved on SDS acrylamide gel and transferred to a nitrocellulose membrane with the Trans-Blot Turbo System (Bio-Rad). Membranes were blocked in 5% non-fat (skim) milk (Difco) in Tris buffered saline with Tween 20 (0.1%) for 1 hour. Membranes were incubated over night with anti SIRT6 rabbit monoclonal antibody (cat-12486, Cell Signaling) or overnight with either Tubulin rabbit monoclonal antibody (cat-2128, Cell Signaling) or HRP-conjugated Beta Actin Monoclonal antibody (cat-HRP-60008, ThermoFisher) in blocking buffer (TBST with 5% w/v with BSA) at 4° C. or Histone H3 (D1H2) XP® Rabbit mAb #4499, 1:1000 in BSA 5%. Membranes were washed 3 times in TBST and were incubated for 1 hour with a secondary anti Rabbit antibody linked to horseradish peroxidase. Immunoreactive bands were detected with the Clarity Western ECL Substrate (Bio-Rad).

### Histological Analysis

The right liver lateral lobe was fixed in 4% Neutral Buffered formaldehyde (NBF). Hematoxylin and eosin (H&E) stains were performed on formalin fixed, paraffin-embedded liver sections at Phatho-Lab Diagnostic LTD, Israel, according to the facility's common practice. Sirius red staining was performed using the Picrosirius red protocol, as known in the art. For liver tissue neutral triglycerides and lipids staining, liver tissues were well-fixed with a formaldehyde-based fixative prior to cryopreservation with sucrose followed by Oil Red O solution staining.

### Example 1: Expression of SIRT6 Protein in a Human Cell Line Following Transfection of SIRT6 mRNA.

Mouse SIRT6 mRNA molecule (defined herein as the native SIRT6 mRNA sequence which corresponds to the open reading frame (ORF)) was synthesized using methods well known in the art.

The sequence of the mouse SIRT6 ORF is defined below as SEQ ID NO: 12:

ATGTCGGTGAATTATGCAGCAGGGTTGTCGCCTTACGCGGATAAG
GGCAAGTGCGGGCTGCCCGAGATCTTCGACCCACCAGAGGAGCTG
GAACGCAAGGTGTGGGAGCTGGCCCGGCTAATGTGGCAGTCCTCC
AGCGTGGTTTTCCACACGGGCGCCGGCATCAGCACCGCCTCTGGC
ATCCCCGACTTCAGAGGCCCCCATGGCGTGTGGACCATGGAGGAA
CGCGGCCTGGCCCCCAAGTTTGACACCACCTTCGAGAATGCTCGG
CCCTCGAAGACCCACATGGCCCTGGTTCAGCTAGAACGCATGGGC
TTCCTCAGCTTCCTGGTCAGCCAGAACGTAGACGGGCTGCACGTG
CGCTCGGGCTTCCCCAGGGACAAGCTGGCAGAGCTGCACGGAAAC
ATGTTTGTAGAGGAATGTCCCAAGTGTAAGACGCAGTACGTCAGA
GACACGGTTGTGGGCACCATGGGCCTCAAGGCCACAGGCCGGCTC
TGCACCGTGGCCAAGACCAGGGGACTTCGGGCCTGTAGAGGGGAG
CTGAGAGACACCATTCTGGACTGGGAGGACTCGTTGCCTGACCGG
GACCTGATGCTCGCTGATGAGGCCAGCAGGACCGCAGACCTGTCT
GTCACCCTGGGTACCTCGCTGCAGATCCGCCCCAGTGGGAACCTG
CCCCTTGCCACTAAGCGCCGAGGAGGCCGTCTGGTCATTGTCAAC
CTGCAACCCACAAAACATGACCGCCAGGCTGACCTGCGCATCCAC
GGCTACGTGGATGAGGTGATGTGCAGACTCATGAAGCATCTGGGG
CTGGAGATTCCAGCCTGGGATGGACCCTGCGTGCTAGACAAAGCC
CTGCCACCTCTGCCTCGCCCAGTAGCACTCAAGGCTGAGCCCCCC
GTGCATCTCAATGGTGCAGTGCATGTTTCGTATAAGTCCAAGCCC
AACAGCCCTATACTCCACAGGCCCCCCAAAAGAGTGAAGACCGAG
GCTGCCCCCAGCTGA

The produced mRNA molecule was Capped (Cap 1) using CleanCap® AG reagent, a 120 PolyA tail was added, and Uridine was substituted with N1-Methyl-Pseudo-U. In addition, the SIRT6 gene 3′UTR was replaced with mouse alpha globin 3′UTR gene.

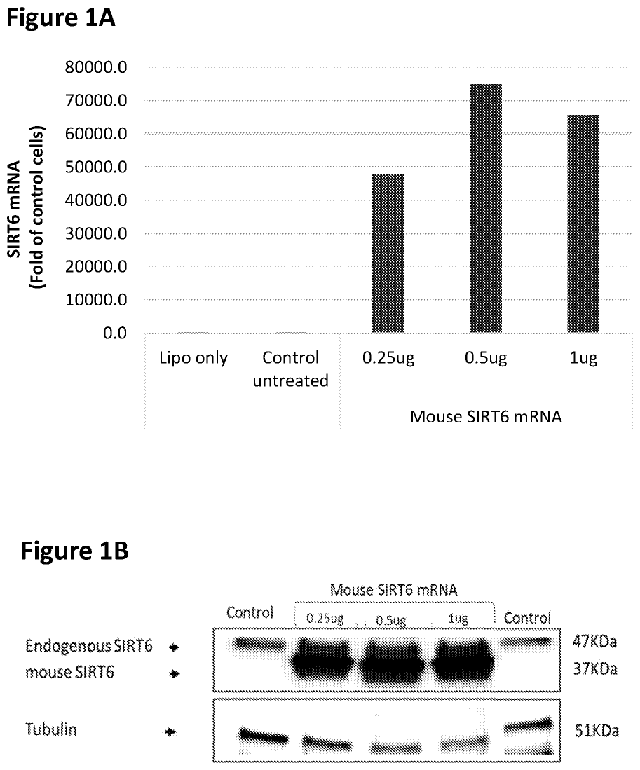

To determine whether introduction of this mRNA sequence to cells results in the expression of the SIRT6 protein, human HEK293 cells were transfected with increasing doses of the mouse SIRT6 mRNA (0.25 μg, 0.5 μg, and 1 μg) using Lipofectamine™ MessengerMAX™ Transfection Reagent, according to manufacturer's protocol. Expression of SIRT6 mRNA and protein levels were evaluated 24 hours after cells transfection using RT-qPCR and Western blot analysis, respectively. Transfection of the cells resulted in increased levels of mouse SIRT6 mRNA (FIG. 1A), and protein (FIG. 1B).

### Example 2: Inducing the Expression of SIRT6 Protein in Mice Liver After Injection of Lipid Nano Particle Formulated Mouse SIRT6 mRNA

Mouse SIRT6 mRNA (as described in Example 1 above) was formulated in D-Lin-MC3-DMA (MC3) formulation. The particles (66 nm, PDI-0.156, mRNA stock concentration 167 μg/mL) were diluted in PBS×1 to the desired concentration and injected, intravenously (iv) by tail vein injection at escalating doses of 0.1, 0.25, or 0.5 mg/kg. Mice injected with the vehicle PBS served as control. Three and six hours after iv injection, liver tissues were extracted from each mouse and SIRT6 mRNA and protein levels were evaluated as described above, using RT-qPCR and Western blot analysis, respectively.

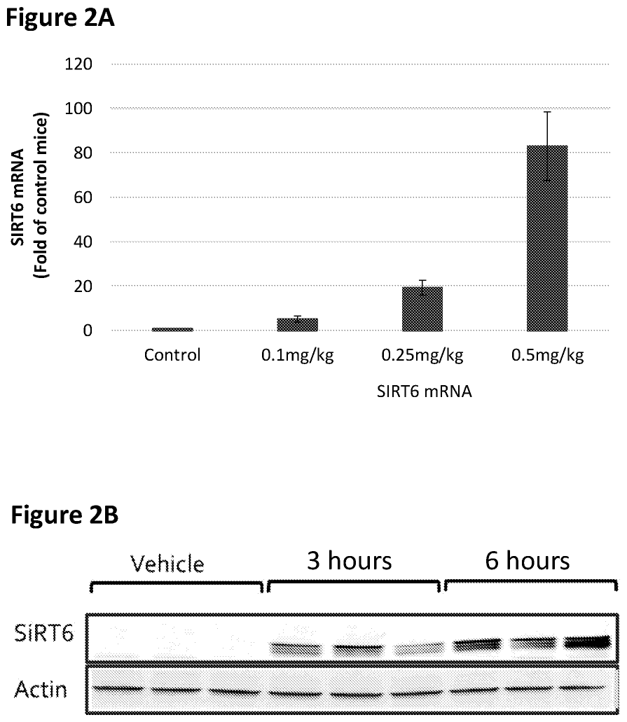

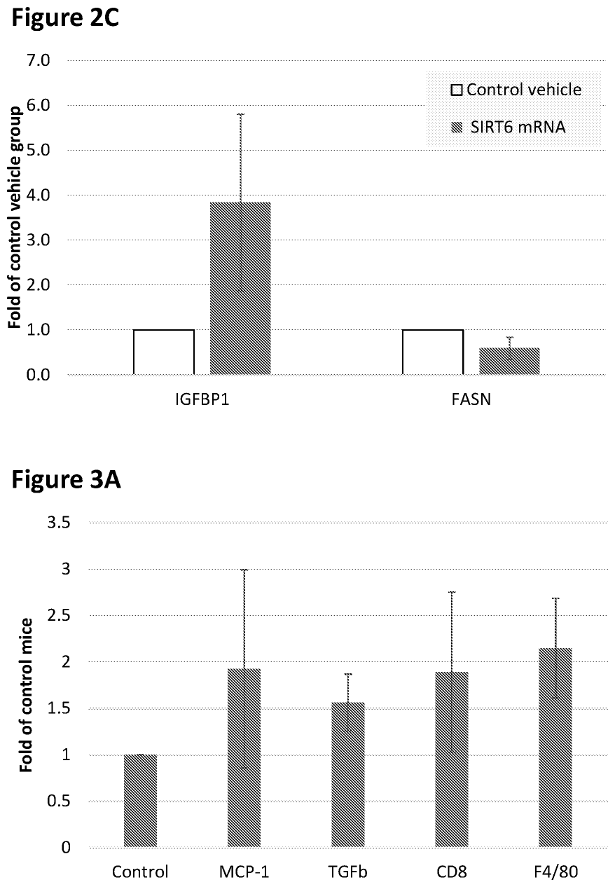

As shown in FIG. 2, increased expression levels of active SIRT6 protein were found in the liver tissue of treated mice. As shown in FIG. 2A, a dose response increase in the expression of SIRT6 mRNA was demonstrated, as evaluated 6 hours after injection. As shown in FIG. 2B, an increased expression of SIRT6 protein was demonstrated 3 and 6 hours after injection of 0.5 mg/kg dose. Increased levels of SIRT6 mRNA and protein were evaluated also after injection of lower doses of 0.1 and 0.25 mg/kg mRNA (data not shown).

It has been shown previously that SIRT6 overexpression results in a significant increase in the expression levels of Insulin growth factor binding protein 1 (IGFBP1) (Elhanaty et al., (2016) Cell reports, Volume 14, Issue 2, Pages 234-242) and reduction of Fatty acid synthase (FASN) (Masri et al., (2014) Cell; 158(3): 659-672) in the liver.

Therefore, to verify the activity of the SIRT6 protein which resulted from the injected SIRT6 mRNA, the expression levels of these genes were tested. As shown in FIG. 2C, elevation of SIRT6 protein in mice liver led to the induction of IGFBP1 expression and to the reduction in the expression of FASN.

### Example 3: Inducing SIRT6 Expression in the Liver in a Mouse Model of NASH

Next, the effects of inducing SIRT6 expression on physiological parameters of liver disease and metabolic dysfunction were assessed in a mouse model of NASH. The following parameters were evaluated: liver weight, ALT/AST levels, liver inflammation, fat accumulation in the liver, fibrosis formation and kidney function.

The SIRT6 mRNA molecule, namely, the modified mRNA transcribed from SEQ ID NO: 12 as described in Example 1, above, formulated in MC3 lipid nano particles was introduced to C57Bl/6 mice on the background of a STAM model, which is a murine model for NASH (Masato Fujii et al., (2013) Med Mol Morphol 46:141-152). C57BL/6J, male, neonatal mice were injected subcutaneously with low dose (200 μg) of streptozotocin (STZ) (Sigma). Beginning four weeks plus 5 days after birth, STZ-primed mice were stimulated with High Fat Diet (HFD) (Research Diet, Cat-D10031901i) continuously, which induced sequential histological changes associated with fatty liver, NASH, and fibrosis. The STZ-primed high fat diet mice are termed herein STZ-HFD mice or NASH mice. SIRT6 mRNA encapsulated in MC-3-LNPs was injected intravenously twice weekly at a dose of 0.5 mg/Kg for 3 and a half weeks prior to sacrifice.

At week 9 of age, blood was collected, liver was extracted from the mice and levels of the liver enzymes ALT/AST, fat accumulation in the liver, serum creatinine, inflammatory gene expression, genes involved in fibrosis as well as staining with H&E, OilRed and Sirius red were performed. Mice injected with PBS were used as control.

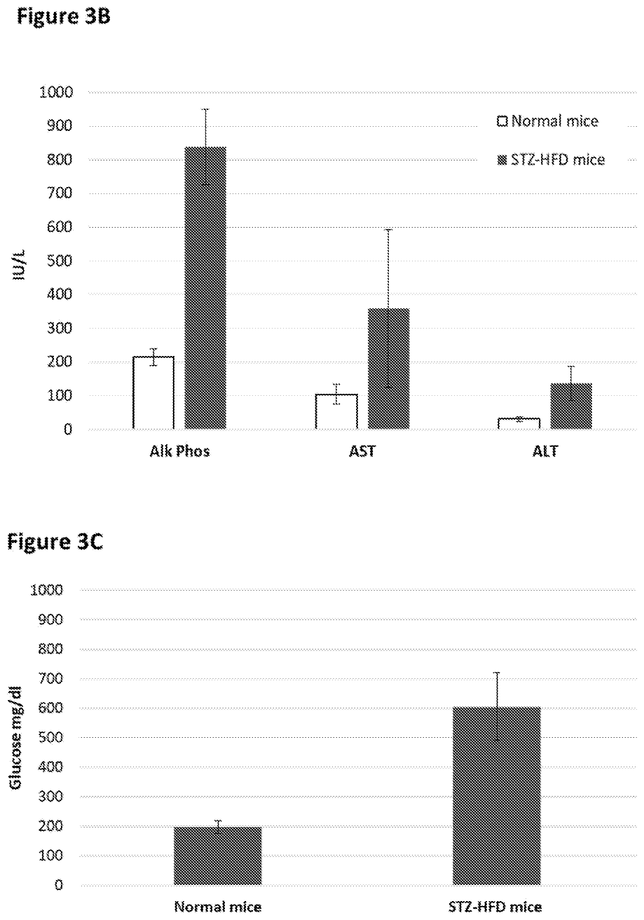

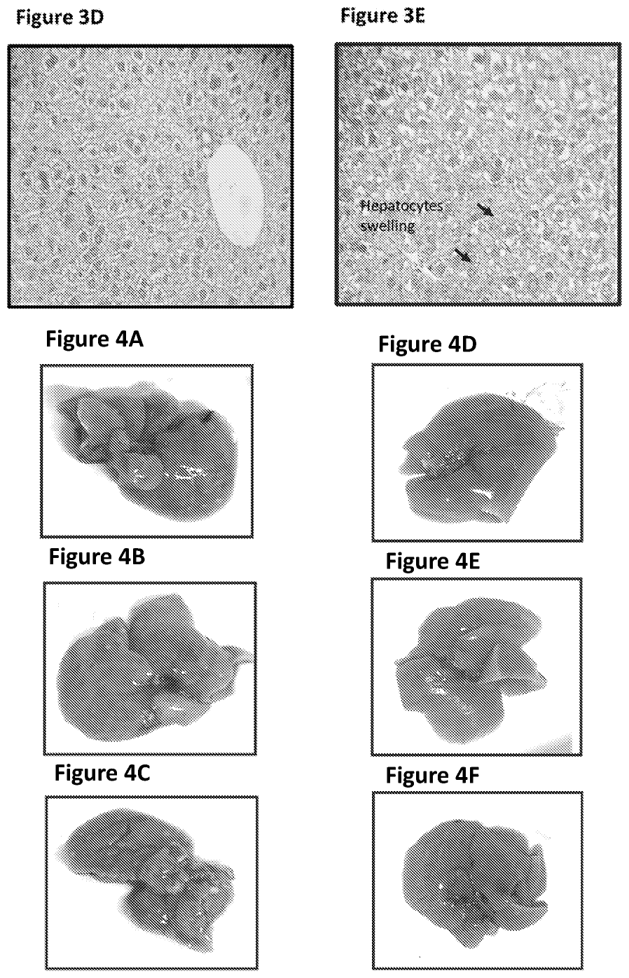

SIRT6 mRNA was introduced to mice when NASH pathology was already observed. Liver inflammation was manifested by the elevation of inflammatory genes (MCP-1 and TGFβ) and the presence of infiltrating lymphocytes and macrophages indicated by the elevated levels of CD8 and F4/80 markers, respectively (FIG. 3A). The expression levels of MCP-1, TGFβ, CD8 and F4/80 were normalized to CycloA gene and were examined by RT-qPCR. Liver toxicity was seen by elevated levels of liver enzymes, AST, ALT, and alkaline phosphatase, in the serum (FIG. 3B). Tolerability to insulin was seen by increased levels of glucose (measured after 16 hours starvation) (FIG. 3C) and presence of hepatocytes swelling in the liver as seen in liver sections after H&E staining (FIGS. 3D and 3E).

At week 9 of age, mice were sacrificed and effects of SIRT6 on NASH pathology was investigated.

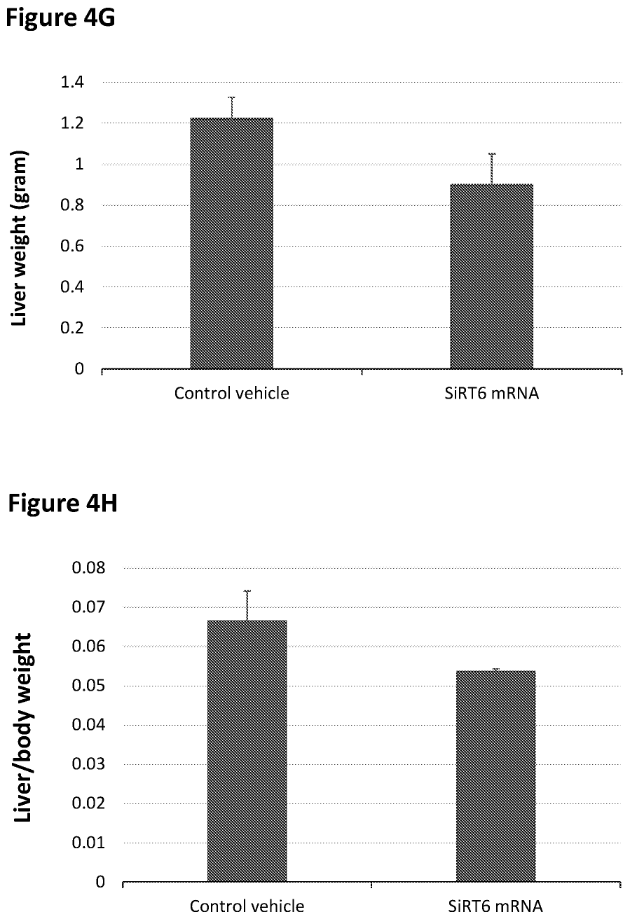

Pale yellow livers, representing accumulation of fat, were seen in the livers of vehicle-treated NASH control mice (FIG. 4A-4C) but not in SIRT6 mRNA treated mice (FIG. 4E-4F) indicating a lower accumulation of fat following treatment with SIRT6 mRNA. Livers of treated mice were healthy in color and non-distended, in marked contrast to the livers of the vehicle-treated control mice. The reduction in liver fat accumulation following SIRT6 mRNA treatment was also manifested by reduced liver weight (FIG. 4G) and the ratio between liver and body weight in SIRT6 mRNA treated mice (FIG. 4H), compared to the control group.

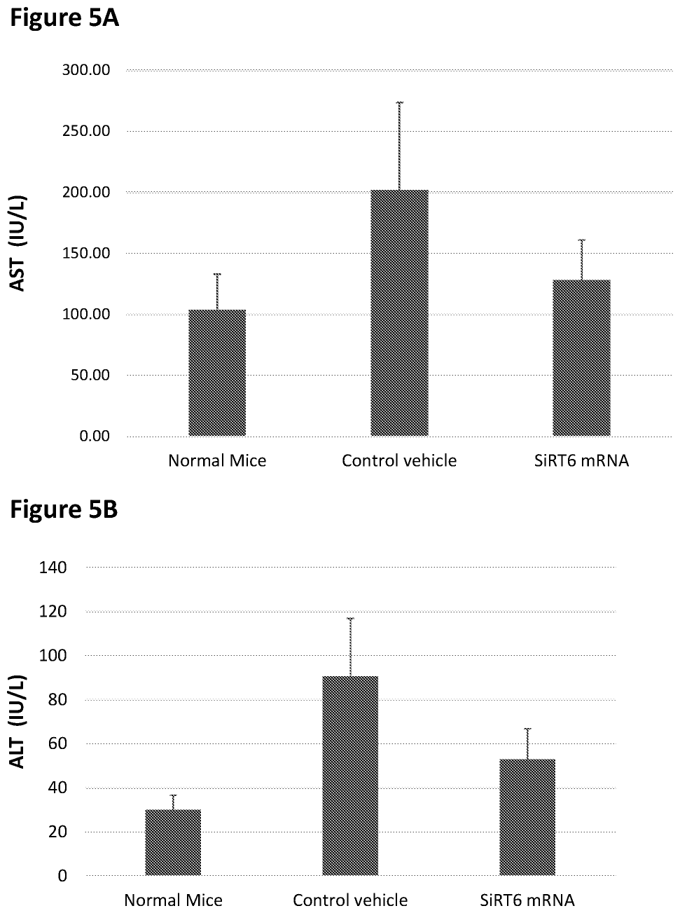

Elevation of serum ALT and AST was detected in STZ-HFD mice, resembling human NASH. Treatment with SIRT6 mRNA resulted in reduced AST (FIG. 5A) and ALT levels (FIG. 5B) in the serum of the treated group compared to control animals.

Hepatocellular ballooning is an important histological parameter in the diagnosis of NASH. It is usually defined, at the light microscopic level, based on H&E staining, as cellular enlargement compared the normal hepatocyte diameter. The swelling of hepatocytes is explained by water accumulation in the cytoplasm as a response to accumulated stress proteins such as heat shock proteins or fat.

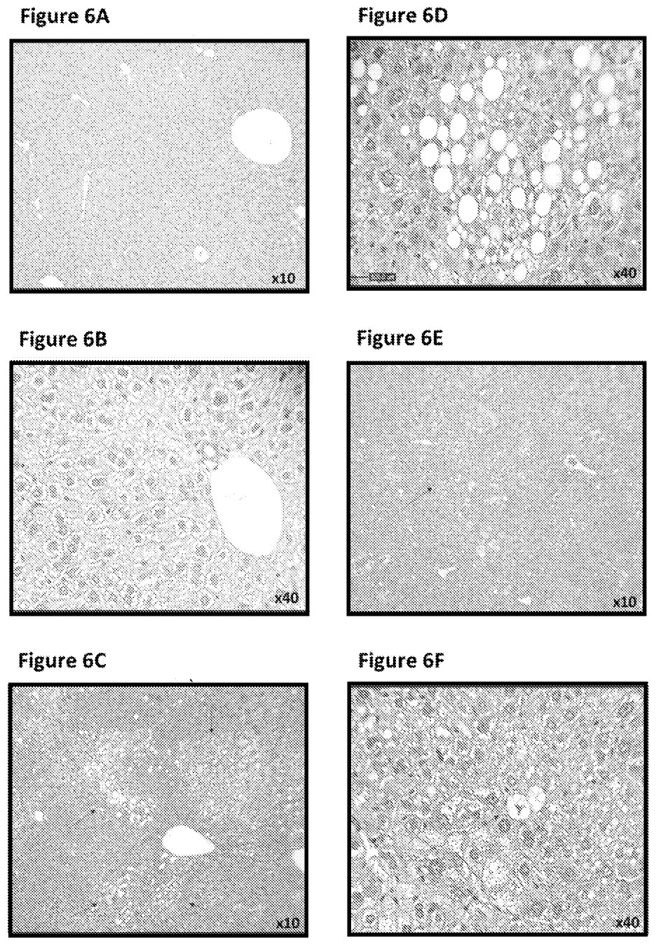

Hepatocytes swelling and ballooning were detectable in STZ-HFD mice at week 9 of age (FIGS. 6C-D) as compared to Normal mice (FIG. 6A-B). Treatment with SIRT6 mRNA reduced the presence of hepatocytes swelling and ballooning (FIG. 6E-F).

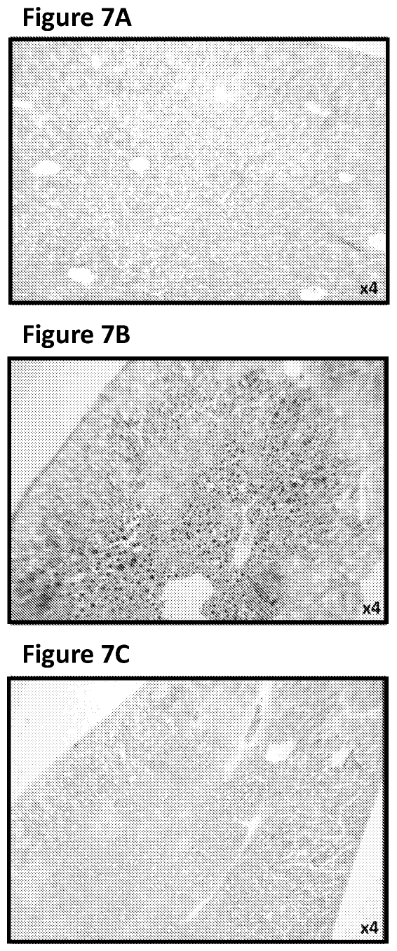

Oil-red staining of neutral triglycerides and lipids in liver hepatocytes showed significant fat deposition, including macro-vesicular fat, in the Vehicle-treated NASH model mice at week 9 of age (FIG. 7B). However, fat deposition was gradually decreased in SIRT6 mRNA treated group (FIG. 7C) resulting in reduced fat accumulation. Normal mice aged 9 weeks fed by Chow diet were used as Control healthy mice (FIG. 7A).

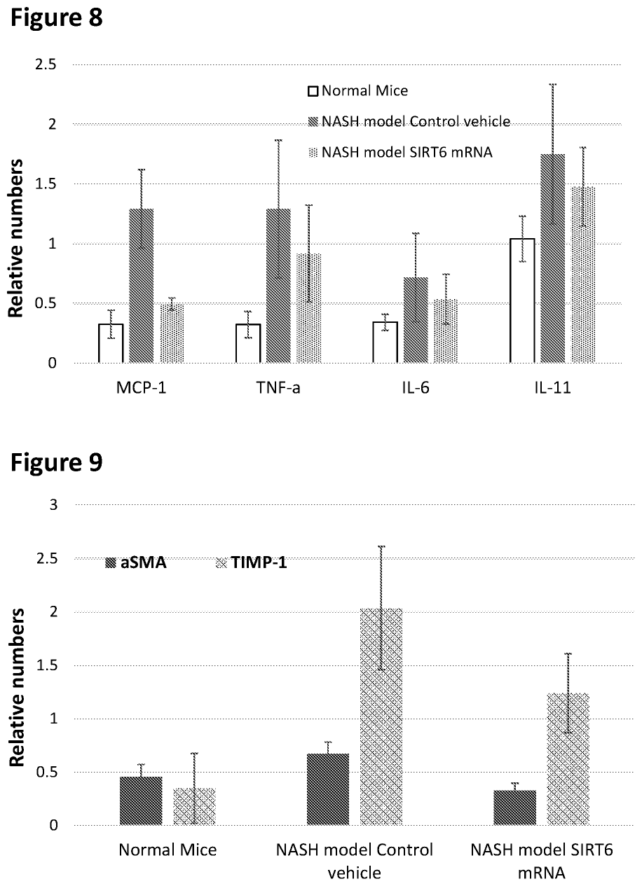

IL-6, TNF-α, MCP-1 and IL-11 genes are up-regulated during the inflammatory phase of NASH. The expression levels of these genes in vehicle-and SIRT6 mRNA-treated mice were examined using RT-qPCR. At 9 weeks of age, expression of these genes was induced in the livers of vehicle-and SIRT6 mRNA-treated STZ-HFD mice. SIRT6 mRNA treatment resulted in significantly reduced expression of the inflammatory markers relative to control treatment in NASH mice (FIG. 8).

Hepatic fibrosis is a pathological process characterized by deposition of extracellular matrix (ECM) proteins. Changes in the ECM are mainly regulated by matrix metalloproteinases (MMPs), which are a family of proteolytic enzymes capable of degrading the ECM. The activity of MMPs is tightly regulated by the amount of active protein and the concentration of specific inhibitors, called tissue inhibitors of metalloproteinases (TIMPs). Extensive studies have identified that TIMPs play a key role in the progression of fibrosis. α-SMA expression is considered a reliable marker of hepatic stellate cell activation and a key biomarker for liver fibrosis.

Formation of fibrotic tissue in the liver of the STZ-HFD mice at week 9 of age was evaluated by measuring TIMP-1 and α-SMA gene expression. As seen in FIG. 9, both genes were induced in SZT-HFD mice compared to Control normal mice. Introduction of SIRT6 mRNA attenuates fibrosis formation as manifested by the reduction in TIMP-1 and α-SMA expression levels. The expression levels of these genes were examined using RT-qPCR.

Accumulating evidence suggests that NASH and Chronic Kidney disease (CKD) share many risk factors and common pathogenetic mechanisms and that NASH is associated with an increased prevalence and incidence of CKD.

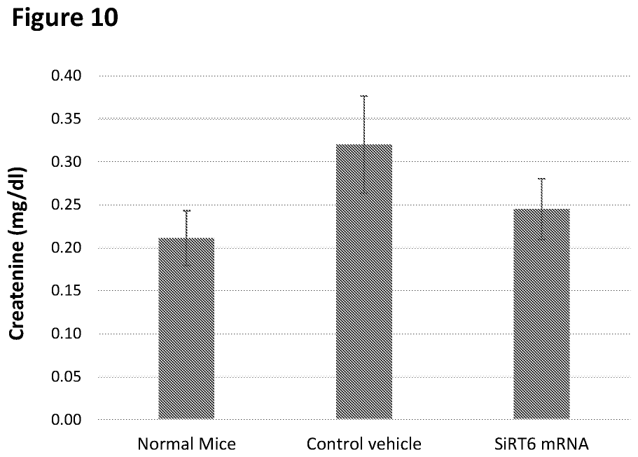

CKD is defined by a low serum creatinine-based estimated glomerular filtration rate (eGFR). STZ-HFD mice at age week 9 show increased levels of Creatinine as compared to their counterpart control mice. Injection of SIRT6 mRNA which results in its increased activity in the liver of STZ-HFD mice causes a 50% reduction in the elevated levels of serum creatinine. Without wishing to be bound by theory, this may be the result of an improved kidney function and better creatinine clearance (FIG. 10).

### Example 4: Induction of SIRT6 Expression in the Liver in a Mouse Model of NASH Significantly Improves Survival

Next, the effects of inducing SIRT6 expression on survival were assessed in the mouse model of NASH (the STAM model as described in Example 3). C57BL/6J, male, neonatal mice were injected subcutaneously with low dose (200 μg) of streptozotocin (STZ) (Sigma). Five weeks after birth, STZ-primed mice were stimulated with High Fat Diet (HFD) (Research Diet, Cat-D10031901i) continuously, which induced sequential histological changes including fatty liver, NASH, and fibrosis. The STZ-primed high fat diet mice are termed herein STZ-HFD. The SIRT6 mRNA molecule, namely, the modified mRNA transcribed from SEQ ID NO: 12 as described in Example 1, above, formulated in MC3 lipid nano particles was introduced to C57Bl/6 mice on the background of a STAM model at week 11 of age. mRNA was injected at a dose of 0.25 mg/Kg twice weekly for 4 weeks followed by once weekly injection for 4 weeks and then bi-weekly injections for additional 8 weeks (N=6) (overall 16 injections in 18 weeks). Mice injected with the vehicle PBS served as control (N=7).

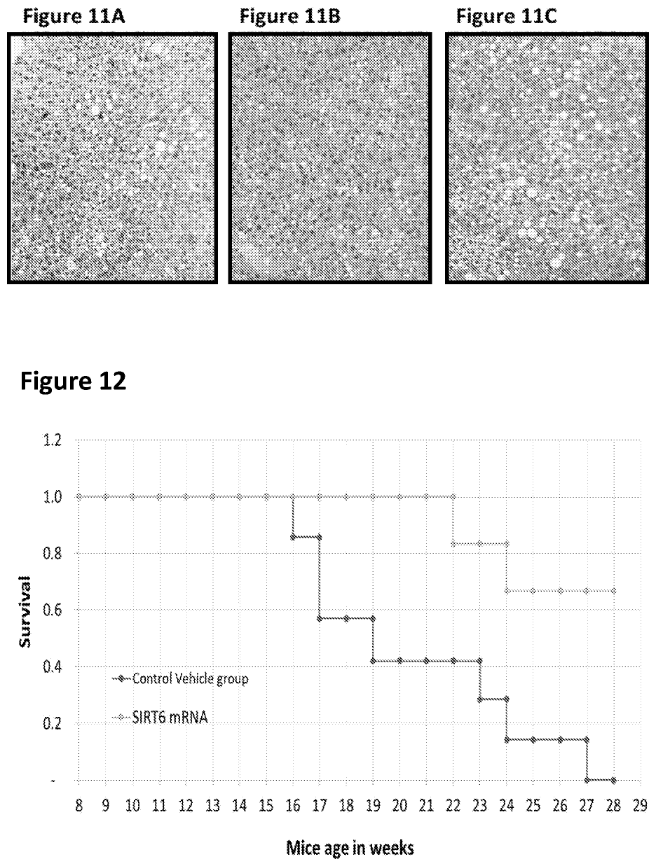

Mice treatment with either PBS or SIRT6 mRNA started at week 11 of age, which corresponds to a liver steatohepatitis stage. Blood sugar was high (450 mg/dl±110) and hepatocyte swelling, and ballooning were detectable, as shown by H&E staining in three representative mice (FIG. 11A-C).

Mice from the Control vehicle group with fatty, early fibrotic, and inflamed liver (STZ-HFD model) who received continuously the HFD started to die at week 16 of age. Approximately 42% of mice died at week 18 and 85% at week 23. At week 27 of age all the mice from the control group had died. On the other hand, all mice from the SIRT6 mRNA treated group survived until week 23 while 16% died at week 23 and 33% died at week 24 (FIG. 12). All remaining mice survived after week 28 of age (two-sided p value 0.0039 HR 0.13, 95% CI [0.026, 0.658]).

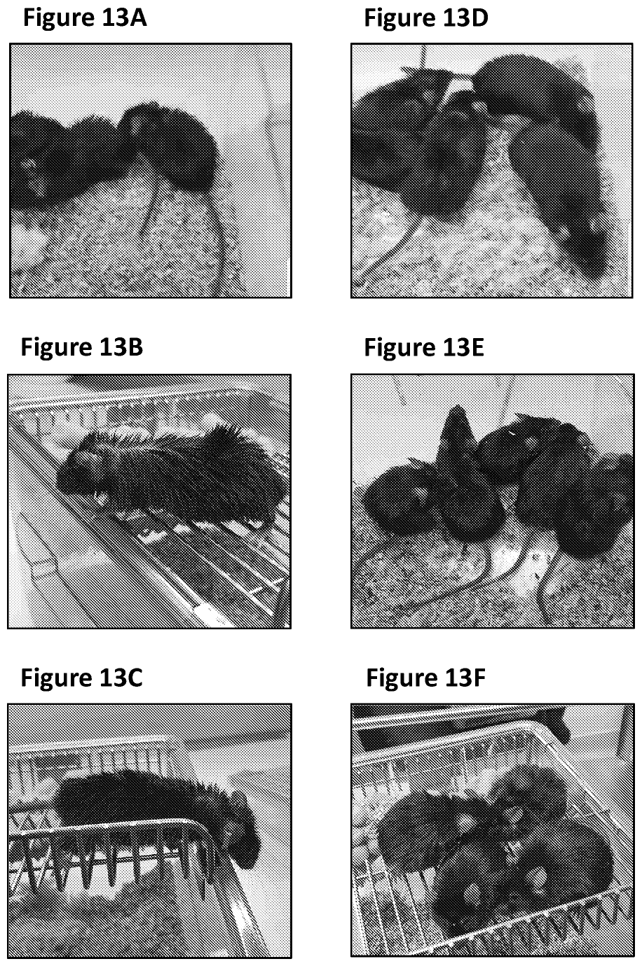

The study was terminated, one week after all control mice died. During this time, the SIRT6 treated mice were full of energy having shining fur compared to the control mice that from week 16 of age looked weak, with oily and stiff fur (FIG. 13). Control mice had severe hyperlipidemia with classic “white, milky” blood. The blood from the SIRT6 -mRNA treated animals was normal in appearance (Not shown). Blood test from the SIRT6 mRNA treated mice at week 28 of age showed significant reduction in blood glucose (382±64 mg/dl) and triglycerides (152±32 mg/dl) levels compared to the last dying mouse at week 27 of age from the control group (776 mg/dl and 868 mg/dl, respectively).

### Example 5: Inducing SIRT6 Expression in the Liver in the MC-Reduced HFD Mouse Model of NASH Improves Multiple Markers of NASH Disease

The effects of inducing SIRT6 expression on liver inflammation, fibrosis formation and kidney function were assessed in an additional mouse model of NASH.

In this model, mice are fed with choline-deficient (CD) reduced Methionine (0.1%) diet (MC-Reduced HFD), which produces a more progressive liver pathology characterized by the development of steatosis with inflammation and fibrosis in rodents within a short time frame (Chiba et al., 2016).

C57BL/6J Male mice at week 10 of age, were fed with L-Amino Acid Diet With 45 kcal % Fat With 0.1% Methionine and No Added Choline (Research Diet, Cat #A06071309i).

The SIRT6 mRNA molecule, namely, the modified mRNA transcribed from SEQ ID NO: 12 as described in Example 1, above, formulated in MC3 lipid nano particles was introduced to C57Bl/6 mice after 7 weeks under the MC-Reduced HFD. mRNA was injected at a dose of 0.25 mg/Kg twice weekly for 4 weeks (N=8). Mice injected with the vehicle PBS served as control (N=8).

SIRT6 mRNA was introduced to mice when NASH pathology could already be observed. Blood was collected, liver was extracted from the mice and levels of the liver enzymes alanine aminotransferase/aspartate aminotransferase (ALT/AST), fat accumulation in the liver and Sirius red staining analyses were performed (N=5).

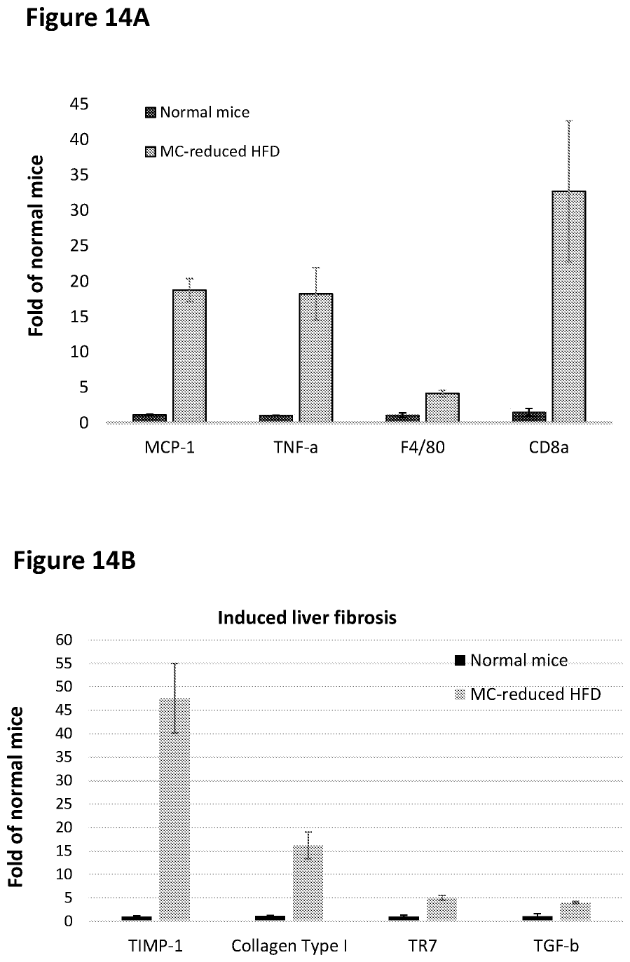

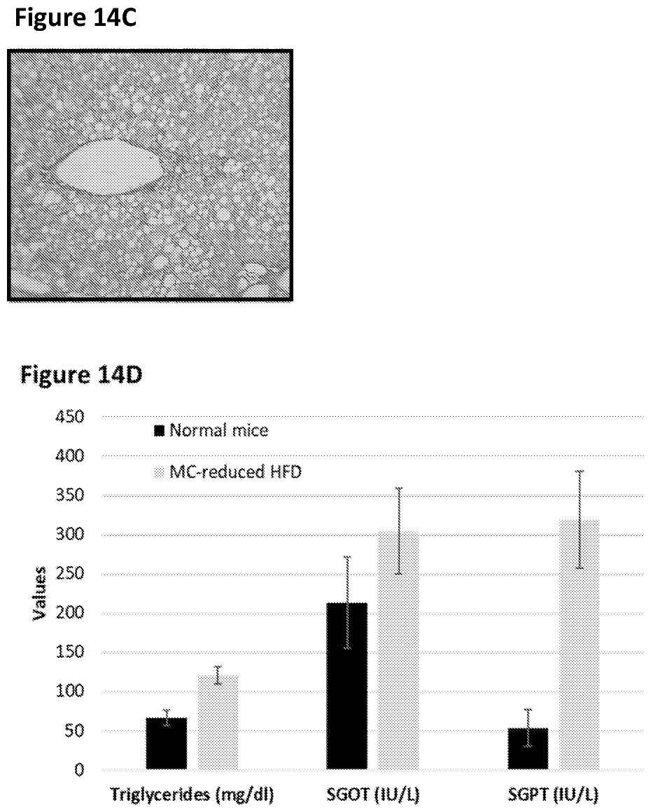

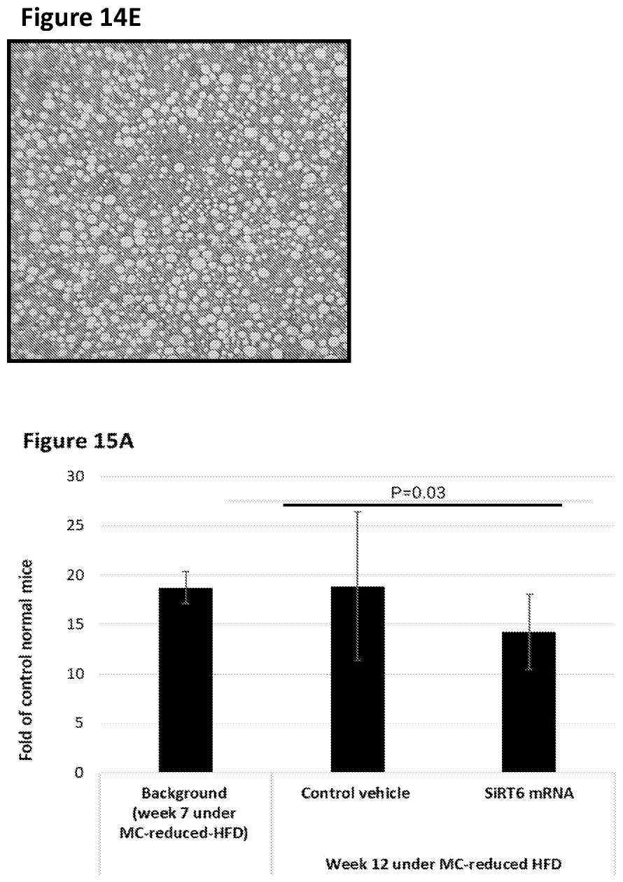

Liver inflammation was manifested by the elevation of inflammatory genes (MCP-1 and TNF-α) and the presence of infiltrating lymphocytes and macrophages indicated by the elevated levels of CD8 and F4/80 markers, respectively (FIG. 14A). Formation of fibrotic tissue was manifested by the elevation of genes involves in fibrosis (TIMP-1, Collagen Type I, TR7 and TGFβ) (FIG. 14B) and by the presence of collagen fibers after Sirius red staining (FIG. 14C). The expression levels of the genes were normalized to the Geo mean of IPO8 & RplPO genes and were examined by RT-qPCR. Liver toxicity was seen by elevated levels of liver enzymes, AST, ALT, in the serum (FIG. 14D). accumulation of fat was seen by measuring the triglycerides level in the serum (FIG. 14D) and the presence of hepatocytes swelling in the liver as seen in liver section after H&E staining (FIG. 14E).

Two weeks after the last injection of SIRT6 mRNA, at week 12 under MC-Reduced HFD, mice were sacrificed and effects of SIRT6 on NASH pathology was investigated.

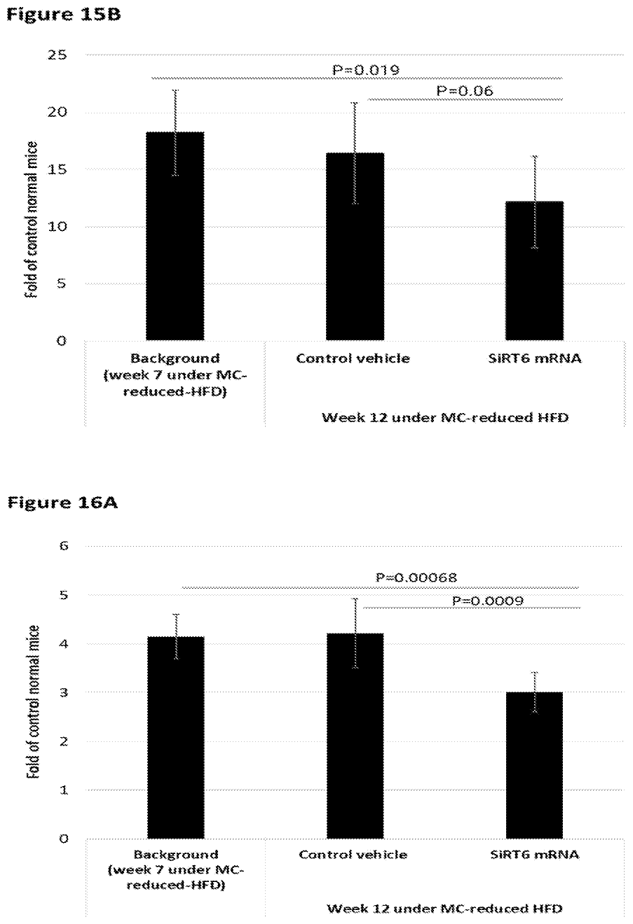

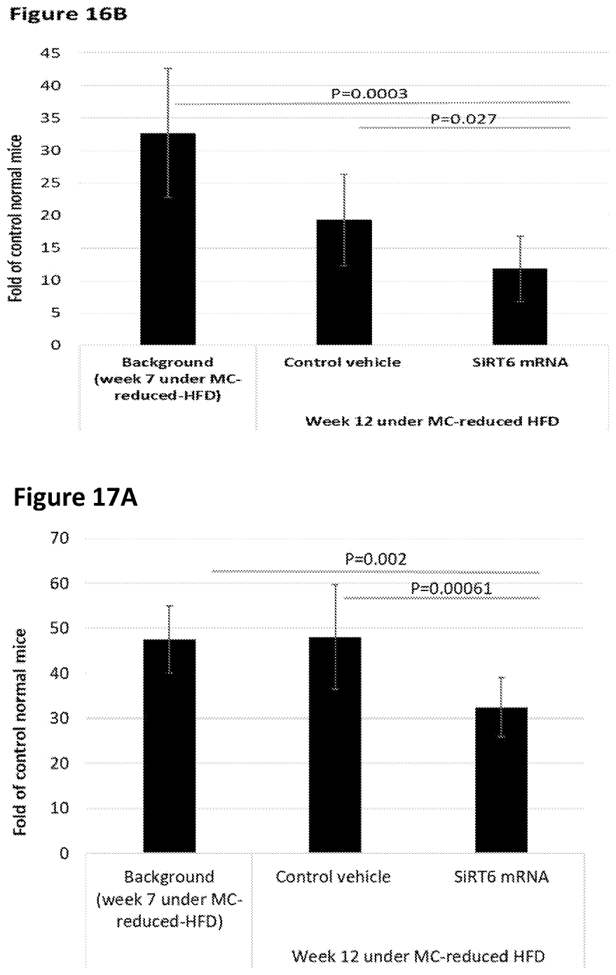

The immune reaction is represented by immune/inflammatory cell infiltrates and secretion of inflammatory cytokines by liver cells. MCP-1 and TNF-α genes are up-regulated during the inflammatory phase of NASH. These genes are induced in mice livers under MC-Reduced HFD and reduced in mice treated with SiRT6 mRNA twice weekly with a 0.25 mg/kg dose for 4 weeks (FIG. 15A and 15B, respectively). At the same time, significant decline in infiltrating macrophages and lymphocytes was seen in liver tissue of mice treated with SiRT6 mRNA compared to Control vehicle group or to the background on which the treatment began, as indicated by the reduced levels of CD8 and F4/80 markers (FIG. 16A and 16B, respectively).

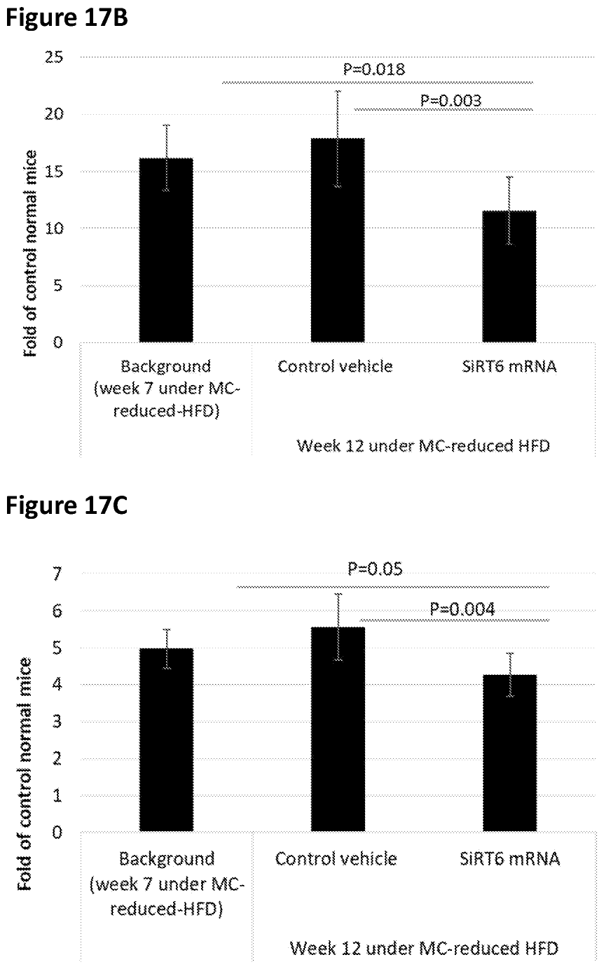

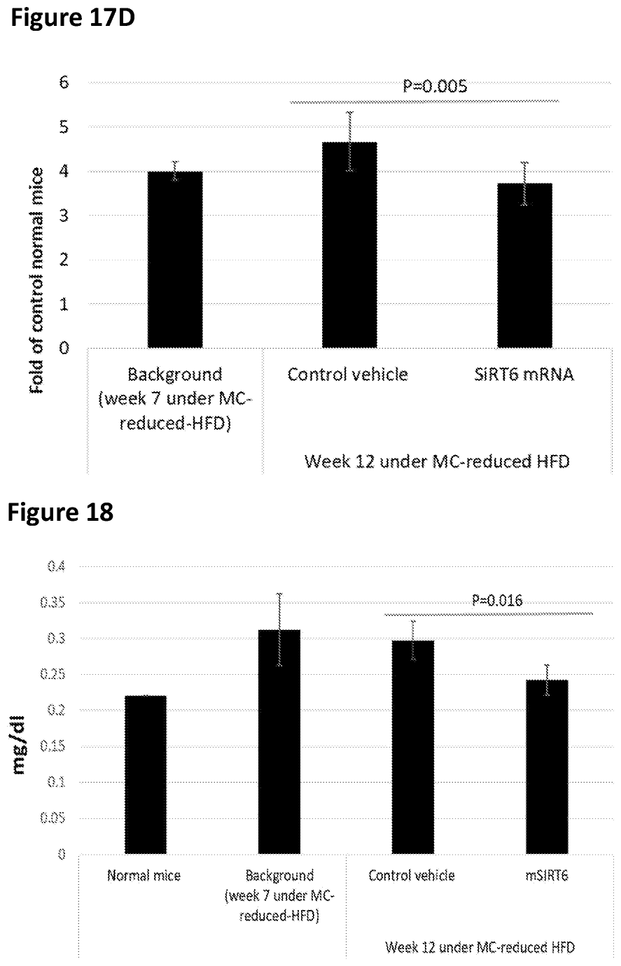

TGF-β is a central regulator in chronic liver disease contributing to all stages of disease progression from initial liver injury through inflammation and fibrosis to cirrhosis and hepatocellular carcinoma. Liver damage-induced levels of active TGF-β enhance hepatocyte destruction and mediate hepatic stellate cell and fibroblast activation. TGF-β levels in the liver were induced under MC-Reduced HFD (FIG. 14B and 17D). Formation of fibrotic tissue in the liver under MC-Reduced HFD was also evaluated by measuring TIMP-1, Collagen Type I and TR7 gene expression. As seen in FIG. 14B, all genes were induced under this diet as compared with Control normal mice. Introduction of SIRT6 mRNA (twice weekly, a 0.25 mg/kg dose for 4 weeks) reduced fibrosis as manifested by the significant reduction of the expression of these genes compared to their levels in the liver at the beginning of the treatment (week 7) or by the end of the study (FIG. 17A-C). The expression levels of all genes were examined using RT-qPCR.

Accumulating evidence suggests that NASH and Chronic Kidney disease (CKD) share many risk factors and common pathogenetic mechanisms and that NASH is associated with an increased prevalence and incidence of CKD.

CKD is defined by a low serum creatinine-based estimated glomerular filtration rate (eGFR). Feeding mice with MC-Reduced HFD increased levels of Creatinine as compared to their counterpart control mice. Injection of SIRT6 mRNA which results in its increased activity in the liver of mice fed with MC-Reduced HFD causes a 20% reduction in the elevated levels of serum creatinine. Without wishing to be bound by theory, this may be the result of an improved kidney function and better creatinine clearance (FIG. 18).

### Example 6: Enhanced Human SIRT6 mRNA Expression, Protein Translation and Stability After Codon Optimization

Next, the expression of human SiRT6 was investigated. To that end, a codon optimized SiRT6 mRNA was prepared.

Codon optimization was implemented to human SIRT6 mRNA transcribed from SEQ ID No. 11. Several codon optimized sequences were prepared, as follows:

Human SIRT6 Codon optimized SEQ ID No: 41
(GC = 59.55%, CAI = 0.81 with self-folding
energy −398.1 kcal/mol):
atgagtgttaactatgctgccggcctgtccccttacgctgataag
gggaagtgtgggctgcccgagattttcgatcccccggaagagctg
gaaagaaaagtgtgggagctggcgagactggtctggcagtctagc
tcagttgtgttccatactggagccggcatcagtacggccagcggg
atcccggatttcagaggcccacacggcgtgtggacaatggaagag
aggggactggcccctaaattcgatactaccttcgagagcgcgaga
ccaacacaaactcacatggccctggtccagctggaacgcgtgggg
ttgctcaggttcctcgtgagtcagaacgtggacggcctgcacgtc
aggagtggtttcccccgtgacaaactggccgagttgcacggcaat
atgttcgtggaagagtgtgctaagtgcaaaacccagtatgtccgc
gatactgtagtcggtacaatgggcctcaaggctaccggccgtctt
tgcactgtggctaaggcgcgcggactgagggcgtgccgcggagag
ctgcgcgataccatcctggactgggaggattccttgcccgacagg
gacctggccctggccgacgaggctagccgcaacgcggatctgtcc
attacactgggtacctccctgcagatccgccctagcggtaacttg
cctcttgccacaaaaagacgtggagggcgtctcgtgatcgtgaat
ctgcagcctaccaaacacgacaggcacgcagacctcagaatccac
ggttacgtggacgaagtgatgactcgtctgatgaagcatttggga
ctggagattcctgcatgggatggcccccgtgtgctggagegggct
ttgccccctctgccccgtcctccaacccccaagctggagcctaaa
gaggaatccccaacccgcatcaacggttctatcccagctggacca
aagcaggagccgtgtgctcaacacaacggcagcgaacccgcctca
cctaagegegaacgccccacctccccagccccacacegccctcca
aaacgcgtgaaagccaaggcagtgccctcctga
Human SIRT6 Codon optimized SEQ ID No: 42
(GC = 53.84%, CAI = 0.72 with
self-folding energy −349.7 kcal/mol):
ATGTCTGTAAATTACGCCGCCGGCCTCTCACCATACGCTGATAAG
GGCAAGTGCGGTCTTCCAGAGATATTTGACCCGCCTGAGGAGCTG
GAGCGAAAAGTCTGGGAACTCGCACGACTTGTATGGCAATCCTCT
TCTGTGGTATTTCACACCGGAGCCGGGATCTCAACTGCCTCTGGA
ATTCCCGATTTCCGCGGTCCCCATGGAGTGTGGACAATGGAAGAA
CGAGGGTTGGCCCCAAAATTTGACACCACCTTTGAATCCGCTCGG
CCTACACAGACTCACATGGCGTTGGTTCAACTCGAGCGGGTTGGG
TTGCTGAGATTTCTCGTGAGTCAAAATGTTGATGGTCTTCATGTG
AGAAGTGGGTTTCCAAGGGACAAATTGGCCGAACTGCATGGGAAC
ATGTTCGTTGAGGAGTGCGCAAAGTGCAAGACACAGTACGTAAGG
GACACCGTTGTTGGGACTATGGGTCTGAAGGCTACTGGACGCCTC
TGCACCGTCGCAAAGGCGAGGGGACTCCGCGCTTGCCGGGGAGAG
CTTCGAGATACAATCCTTGACTGGGAAGATTCACTCCCTGATAGA
GATCTCGCGTTGGCTGACGAGGCATCTCGGAATGCAGACCTTTCC
ATCACCTTGGGTACTTCTCTTCAAATTCGGCCATCAGGAAATCTT
CCACTTGCTACAAAAAGACGGGGTGGTCGCCTTGTGATTGTAAAC
CTGCAGCCTACTAAACATGATCGGCACGCTGATCTCAGGATCCAT
GGTTATGTTGATGAAGTAATGACGCGACTGATGAAGCATCTCGGT
CTCGAAATACCCGCTTGGGACGGGCCTAGGGTACTCGAGAGAGCC
CTTCCCCCACTGCCGAGACCACCGACCCCTAAACTCGAACCAAAG
GAGGAGTCCCCAACCAGGATTAACGGTAGCATTCCCGCCGGACCC
AAACAGGAACCGTGCGCTCAACACAACGGTTCAGAACCGGCGTCT
CCTAAAAGAGAGCGACCAACCTCTCCGGCGCCTCACCGCCCCCCT
AAAAGGGTCAAGGCAAAAGCAGTACCTTCATGA
Human SIRT6 Codon optimized SEQ ID No: 43
(GC-63.20%, CAI = 0.94 with
self-folding energy −398.1 kcal/mol):
ATGAGCGTGAACTACGCCGCCGGCCTGAGCCCCTACGCCGACAAG
GGCAAGTGCGGCCTGCCCGAGATCTTCGACCCCCCCGAGGAGCTG
GAGAGAAAGGTGTGGGAGCTGGCTAGACTGGTGTGGCAGAGCAGC
AGCGTGGTGTTCCACACCGGCGCCGGCATCTCCACCGCGAGCGGT
ATTCCCGACTTCAGAGGCCCCCACGGCGTGTGGACCATGGAGGAG
AGAGGCCTGGCCCCCAAGTTCGACACCACCTTCGAGAGCGCTAGA
CCCACACAGACCCACATGGCCCTGGTGCAGCTGGAGAGAGTGGGC
CTGCTGAGATTCCTGGTGTCTCAGAACGTGGACGGCCTGCACGTG
AGAAGCGGCTTCCCTAGAGACAAGCTGGCCGAGCTGCACGGCAAC
ATGTTCGTGGAGGAGTGCGCCAAGTGCAAGACACAGTACGTGAGA
GACACCGTGGTGGGCACCATGGGCCTGAAGGCCACCGGCAGACTG
TGCACCGTGGCCAAGGCTAGAGGCCTGAGAGCCTGCAGAGGCGAG
CTGAGAGACACCATCCTGGACTGGGAGGACAGCCTGCCCGACAGA
GACCTGGCCCTGGCCGACGAGGCTAGCAGAAATGCCGACTTAAGC
ATTACCCTGGGCACAAGCCTGCAGATCAGACCTAGCGGCAACCTG
CCCCTGGCCACCAAGAGAAGAGGCGGCAGACTGGTGATCGTGAAC
CTGCAGCCCACCAAGCACGACAGACATGCAGACCTGAGAATCCAC
GGCTACGTGGACGAGGTGATGACAAGACTGATGAAGCACCTGGGG
CTTGAGATCCCCGCATGGGATGGACCTAGAGTGCTGGAGAGAGCC
TTACCCCCCTTGCCGAGACCTCCCACACCCAAGCTGGAGCCCAAG
GAGGAGAGCCCCACCCGCATCAATGGAAGCATCCCCGCTGGGCCC
AAGCAAGAGCCCTGTGCTCAGCACAATGGCAGCGAGCCCGCTAGC
CCGAAACGGGAGAGACCAACGTCACCTGCCCCCCACAGACCCCCC
AAGAGAGTGAAGGCCAAGGCCGTGCCTAGCTGA
Human SIRT6 Codon optimized SEQ ID No: 44
(GC-61.61%, CAI-0.93 with
self-folding energy −414.1 kcal/mol):
ATGAGCGTGAACTATGCCGCTGGCCTGAGCCCCTACGCCGACAAG
GGCAAGTGCGGCCTGCCTGAGATCTTTGACCCCCCCGAGGAACTG
GAGAGAAAGGTGTGGGAGCTGGCCCGGCTGGTCTGGCAGAGCAGC
AGCGTGGTGTTCCACACAGGCGCCGGAATCAGCACCGCCAGCGGC
ATTCCTGACTTCAGAGGCCCTCACGGCGTGTGGACCATGGAAGAG
AGAGGCCTTGCCCCTAAATTCGACACCACATTCGAGAGCGCCAGA
CCTACACAGACACACATGGCCCTGGTCCAGCTGGAGCGGGTGGGC
CTGCTGCGGTTCCTGGTGTCCCAGAACGTGGACGGCCTCCATGTG
CGGAGCGGCTTCCCCAGAGATAAGCTGGCCGAGCTGCACGGAAAT
ATGTTTGTGGAAGAATGTGCTAAGTGCAAGACCCAGTACGTGAGA
GATACAGTGGTCGGCACCATGGGCCTGAAGGCCACCGGCAGACTG
TGCACCGTGGCCAAAGCCAGAGGACTCAGGGCATGCAGAGGAGAG
CTGAGAGACACCATCCTGGACTGGGAAGATAGCCTGCCAGATCGG
GACCTGGCTCTGGCCGATGAGGCCTCTAGAAACGCCGATCTGTCC
ATCACCCTGGGAACATCTCTGCAAATCAGACCCTCTGGCAACCTG
CCTCTGGCTACAAAGCGGAGAGGAGGCAGACTCGTGATCGTGAAT
CTGCAGCCTACCAAGCACGACAGACACGCCGACCTGCGCATCCAC
GGCTACGTGGACGAGGTGATGACCCGGCTGATGAAACACCTGGGC
CTGGAGATCCCTGCCTGGGACGGCCCCAGAGTTCTGGAAAGAGCT
CTGCCTCCTCTGCCCAGACCTCCAACCCCTAAACTGGAACCCAAG
GAAGAGAGCCCTACGCGGATCAACGGCAGCATCCCGGCCGGCCCC
AAGCAGGAGCCATGTGCCCAGCACAACGGCAGCGAGCCTGCCTCC
CCAAAGCGGGAACGGCCTACCAGCCCTGCTCCTCACAGACCACCT
AAGAGGGTGAAGGCCAAGGCCGTGCCTTCTTGA
Human SIRT6 Codon optimized SEQ ID No: 45
(GC = 58.80%, CAI = 0.93 with
self-folding energy −378.8 kcal/mol):
ATGAGCGTGAACTATGCCGCCGGACTGAGCCCCTATGCCGATAAG
GGAAAGTGTGGCCTGCCTGAGATCTTCGACCCTCCTGAGGAACTG
GAAAGAAAAGTGTGGGAACTCGCCCGGCTCGTGTGGCAGAGTAGC
TCTGTGGTGTTTCACACCGGCGCTGGCATCTCTACAGCCAGCGGC
ATCCCTGATTTCAGAGGACCTCATGGCGTGTGGACCATGGAAGAG
AGAGGACTGGCCCCAAAGTTCGACACCACCTTCGAGAGCGCCAGA
CCTACACAGACACACATGGCCCTGGTGCAGCTGGAAAGAGTGGGA
CTGCTGAGATTCCTGGTGTCCCAGAATGTGGACGGACTGCATGTG
CGGAGCGGCTTCCCTAGAGATAAGCTGGCCGAGCTGCACGGCAAC
ATGTTCGTGGAAGAGTGCGCCAAGTGCAAGACCCAGTACGTGCGG
GATACAGTCGTGGGCACAATGGGCCTGAAAGCCACCGGCAGACTG
TGCACTGTGGCCAAAGCTAGAGGCCTGAGAGCCTGTAGAGGCGAG
CTGAGAGACACCATCCTGGACTGGGAAGATAGCCTGCCAGACAGA
GATCTGGCCCTGGCCGATGAGGCCAGCAGAAATGCCGATCTGAGC
ATCACCCTGGGCACCAGCCTGCAGATCAGACCATCTGGCAATCTG
CCTCTGGCCACCAAGAGAAGAGGCGGCAGACTCGTGATCGTGAAC
CTGCAGCCTACCAAGCACGACAGACACGCCGACCTGAGAATCCAC
GGCTACGTGGACGAAGTGATGACCCGGCTGATGAAGCACCTGGGC
CTCGAAATTCCAGCCTGGGATGGACCCAGAGTGCTGGAAAGGGCA
CTTCCTCCACTGCCTAGACCTCCAACACCTAAGCTGGAACCCAAA
GAGGAAAGCCCCACCAGAATCAACGGCAGCATTCCCGCCGGACCT
AAGCAAGAGCCTTGCGCTCAGCACAATGGCAGCGAACCTGCTAGC
CCTAAGAGAGAGAGGCCTACAAGCCCCGCTCCTCACAGACCACCA
AAGAGAGTGAAGGCCAAGGCCGTGCCTAGCTGA
Human SIRT6 Codon optimized SEQ ID No: 46
(GC = 59.74%, CAI = 0.90 with
self-folding energy −402.3 kcal/mol):
ATGAGCGTGAATTACGCTGCCGGCCTGAGTCCTTATGCTGACAAG
GGCAAGTGCGGCCTGCCTGAAATCTTCGACCCTCCTGAGGAGCTG
GAGCGTAAAGTGTGGGAGCTGGCTAGGCTGGTGTGGCAGTCTAGC
TCTGTGGTGTTTCACACCGGCGCCGGTATCTCCACCGCCAGCGGA
ATCCCTGATTTCAGAGGCCCACACGGCGTCTGGACCATGGAAGAG
AGAGGATTAGCCCCAAAATTCGACACCACCTTCGAGAGTGCCAGA
CCTACACAGACCCACATGGCCCTGGTGCAGCTGGAAAGGGTCGGC
CTGCTGAGGTTCCTGGTGAGCCAGAACGTGGATGGCCTTCATGTG
AGGAGCGGCTTTCCAAGAGATAAGCTGGCCGAGCTGCACGGGAAT
ATGTTCGTGGAGGAGTGCGCCAAGTGCAAGACACAGTACGTGAGG
GATACAGTCGTGGGGACTATGGGCCTGAAAGCAACTGGAAGGCTG
TGCACAGTGGCCAAGGCTAGAGGCCTCCGCGCCTGCAGGGGTGAG
CTGAGAGATACCATCCTGGATTGGGAAGACTCTCTGCCTGACAGA
GACCTGGCCCTGGCTGACGAAGCCTCTCGGAATGCCGATCTCAGC
ATCACCCTGGGAACCAGCCTGCAGATCAGACCTTCCGGCAACCTG
CCCCTGGCTACCAAAAGACGCGGAGGAAGACTGGTGATAGTGAAC
CTGCAGCCTACCAAGCACGACAGACACGCAGACCTGCGGATCCAC
GGCTACGTGGACGAGGTTATGACTAGGCTGATGAAACACCTGGGC
CTGGAGATCCCAGCCTGGGATGGACCCAGAGTGCTGGAGCGCGCC
CTGCCACCCCTGCCCAGGCCCCCAACACCTAAGCTTGAGCCAAAG
GAGGAATCTCCAACCCGGATTAATGGCTCTATCCCAGCCGGCCCC
AAGCAGGAGCCATGTGCCCAGCACAACGGCAGTGAGCCCGCCAGT
CCCAAGAGAGAACGGCCAACCTCACCAGCCCCACACCGGCCACCT
AAAAGGGTGAAAGCTAAAGCCGTGCCAAGCTGA
Human SIRT6 Codon optimized SEQ ID No: 47
(CAI: 0.82; GC content: 55.96%
with self-folding energy −397.3 kcal/mol):
ATGTCTGTGAATTACGCTGCCGGGCTCTCACCATACGCAGATAAG
GGTAAGTGCGGTCTGCCAGAGATTTTCGATCCTCCTGAGGAATTG
GAGCGAAAGGTGTGGGAGCTTGCACGCCTCGTTTGGCAGAGCAGC
AGCGTGGTCTTTCATACCGGAGCCGGAATATCTACTGCAAGCGGA
ATCCCGGATTTCCGAGGTCCACACGGGGTGTGGACCATGGAGGAA
AGGGGGTTGGCACCAAAGTTCGACACAACATTCGAGAGCGCTAGA
CCTACCCAGACTCACATGGCTCTGGTGCAGCTCGAGAGAGTTGGC
CTGCTCCGATTCCTCGTGAGCCAGAATGTGGATGGACTTCACGTA
CGAAGCGGCTTTCCTCGGGATAAACTGGCTGAGTTGCACGGGAAC
ATGTTTGTGGAGGAGTGCGCTAAGTGTAAGACCCAGTACGTGCGC
GATACCGTCGTGGGCACCATGGGTCTCAAGGCAACTGGCAGACTG
TGTACAGTTGCTAAGGCACGGGGACTGAGGGCATGTAGAGGAGAG
CTGCGCGATACTATCCTGGACTGGGAAGACAGCCTCCCTGATAGA
GATCTCGCCCTGGCTGACGAGGCCTCTAGGAATGCCGACCTCTCC
ATCACGCTCGGCACTAGCCTGCAGATCCGCCCATCCGGAAACCTC
CCTCTTGCTACCAAGAGAAGAGGAGGACGACTTGTGATCGTCAAC
CTGCAGCCAACTAAGCACGACAGGCACGCCGATCTCAGAATCCAC
GGGTATGTGGATGAGGTCATGACACGGTTGATGAAGCATTTGGGG
TTGGAAATTCCTGCATGGGACGGACCAAGGGTCCTTGAGAGAGCT
CTGCCACCTTTGCCTAGGCCTCCTACTCCCAAGTTGGAACCGAAA
GAAGAATCCCCTACTAGAATCAACGGCTCCATTCCTGCTGGACCT
AAGCAGGAACCCTGTGCTCAGCACAATGGATCAGAACCAGCGTCC
CCTAAAAGAGAGAGGCCCACCAGCCCTGCTCCTCACAGACCACCT
AAACGCGTGAAGGCTAAGGCTGTCCCAAGTTGA
Human SIRT6 Codon optimized SEQ ID No: 48
(CAI: 0.956; GC-Content: 72.65% with
self-folding energy −469.6 kcal/mol):
ATGAGCGTGAACTACGCCGCCGGCCTGAGCCCCTACGCCGACAAG
GGCAAGTGCGGCCTGCCCGAGATCTTCGACCCCCCCGAGGAGCTG
GAGCGCAAGGTGTGGGAGCTGGCCCGCCTGGTGTGGCAGAGCAGC
AGCGTGGTGTTCCACACCGGCGCCGGCATCAGCACCGCCAGCGGC
ATCCCCGACTTCCGCGGCCCCCACGGCGTGTGGACCATGGAGGAG
CGCGGCCTGGCCCCCAAGTTCGACACCACCTTCGAGAGCGCCCGC
CCCACCCAGACCCACATGGCCCTGGTGCAGCTGGAGCGCGTGGGC
CTGCTGCGCTTCCTGGTGAGCCAGAACGTGGACGGCCTGCACGTG
CGCAGCGGCTTCCCCCGCGACAAGCTGGCCGAGCTGCACGGCAAC
ATGTTCGTGGAGGAGTGCGCCAAGTGCAAGACCCAGTACGTGCGC
GACACCGTGGTGGGCACCATGGGCCTGAAGGCCACCGGCCGCCTG
TGCACCGTGGCCAAGGCCCGCGGCCTGCGCGCCTGCCGCGGCGAG
CTGCGCGACACCATCCTGGACTGGGAGGACAGCCTGCCCGACCGC
GACCTGGCCCTGGCCGACGAGGCCAGCCGCAACGCCGACCTGAGC
ATCACCCTGGGCACCAGCCTGCAGATCCGCCCCAGCGGCAACCTG
CCCCTGGCCACCAAGCGCCGCGGCGGCCGCCTGGTGATCGTGAAC
CTGCAGCCCACCAAGCACGACCGCCACGCCGACCTGCGCATCCAC
GGCTACGTGGACGAGGTGATGACCCGCCTGATGAAGCACCTGGGC
CTGGAGATCCCCGCCTGGGACGGCCCCCGCGTGCTGGAGCGCGCC
CTGCCCCCCCTGCCCCGCCCCCCCACCCCCAAGCTGGAGCCCAAG
GAGGAGAGCCCCACCCGCATCAACGGCAGCATCCCCGCCGGCCCC
AAGCAGGAGCCCTGCGCCCAGCACAACGGCAGCGAGCCCGCCAGC
CCCAAGCGCGAGCGCCCCACCAGCCCCGCCCCCCACCGCCCCCCC
AAGCGCGTGAAGGCCAAGGCCGTGCCCAGCTAA
Human SIRT6 Codon optimized SEQ ID No: 49
(GC = 64.61%, CAI = 0.96 with self-folding
energy −416.00 kcal/mol):
ATGAGCGTGAATTACGCCGCCGGCCTGAGCCCCTACGCCGATAAG
GGCAAGTGCGGCCTGCCTGAGATCTTCGACCCCCCTGAGGAGCTG
GAGAGAAAAGTGTGGGAGCTGGCGCGGCTGGTGTGGCAGAGCAGC
AGCGTGGTGTTCCACACCGGCGCCGGCATCTCCACCGCCAGCGGC
ATCCCTGATTTCAGAGGCCCCCACGGCGTGTGGACCATGGAAGAG
AGAGGACTGGCCCCAAAGTTCGACACCACCTTCGAGAGCGCCAGA
CCTACACAGACCCACATGGCCCTGGTGCAGCTGGAGAGAGTGGGC
CTGCTGAGATTCCTGGTGAGCCAGAACGTGGACGGCCTGCATGTG
CGAAGCGGCTTCCCCAGAGACAAGCTGGCCGAGCTGCACGGCAAC
ATGTTCGTGGAGGAGTGCGCCAAGTGCAAGACCCAGTACGTGCGA
GATACCGTCGTGGGCACCATGGGCCTGAAGGCCACCGGCAGACTG
TGCACCGTGGCCAAGGCTAGAGGACTGAGGGCCTGCAGAGGAGAG
CTGAGAGACACCATCCTGGACTGGGAAGACAGCCTGCCCGACAGA
GACCTGGCCCTGGCCGACGAGGCCAGCAGGAATGCCGACCTGTCC
ATCACCCTGGGCACCAGCCTGCAGATCCGACCATCCGGCAACCTG
CCTCTGGCTACCAAGAGACGAGGAGGCCGACTGGTGATCGTGAAC
CTGCAGCCTACCAAGCACGACAGACACGCCGACCTGAGAATCCAC
GGCTACGTGGACGAGGTGATGACCCGGCTGATGAAGCACCTGGGC
CTGGAGATCCCCGCCTGGGATGGACCCAGAGTGCTGGAGAGAGCC
CTGCCACCCCTGCCCAGACCTCCAACCCCCAAGCTGGAGCCCAAG
GAGGAATCCCCCACCCGGATCAACGGCAGCATCCCCGCCGGACCC
AAGCAGGAGCCCTGTGCTCAGCACAACGGCAGCGAGCCCGCCAGC
CCTAAGCGAGAGCGGCCCACCAGCCCTGCCCCTCACAGACCACCT
AAAAGGGTGAAGGCCAAGGCCGTCCCTAGCTGA

Codon Adaptation Index (CAI) measures the synonymous codon usage bias for a DNA or RNA sequence. The CAI quantifies the similarity between the synonymous codon usage of a gene and the synonymous codon frequency of a reference set.

Stability and functionality were compared between the transcribed human SIRT6 SEQ ID NO: 11, SEQ ID NO: 45, SEQ ID NO:48 and SEQ ID NO: 49. As used throughout the Examples, when referring to SEQ ID Nos 11, and 41-49, it is meant the mRNA molecules transcribed from these sequences, namely mRNA molecules having these sequences with uridine bases instead of thymine bases. The different sequences were introduced by cloning into expression vectors. Each of the vectors included similar critical elements that enable efficient in vitro translation in a human cell-free system (same 5′UTR including Kozak consensus sequence, 3′UTR sequences and polyadenosine tail (poly (A) tail) of 80 adenosine ribonucleotides at the 3′-end). a T7 bacteriophage RNA polymerase was chosen for transcription in the Human (In vitro transcription) IVT System. The plasmids were designed to be suitable for Capped (Cap 1) RNA using CleanCap® AG. Also, to improve mRNA stability, translational properties and immunogenicity, uridines were substituted with the chemically modified N1-Methylpseudouridine-5′-Triphosphate.

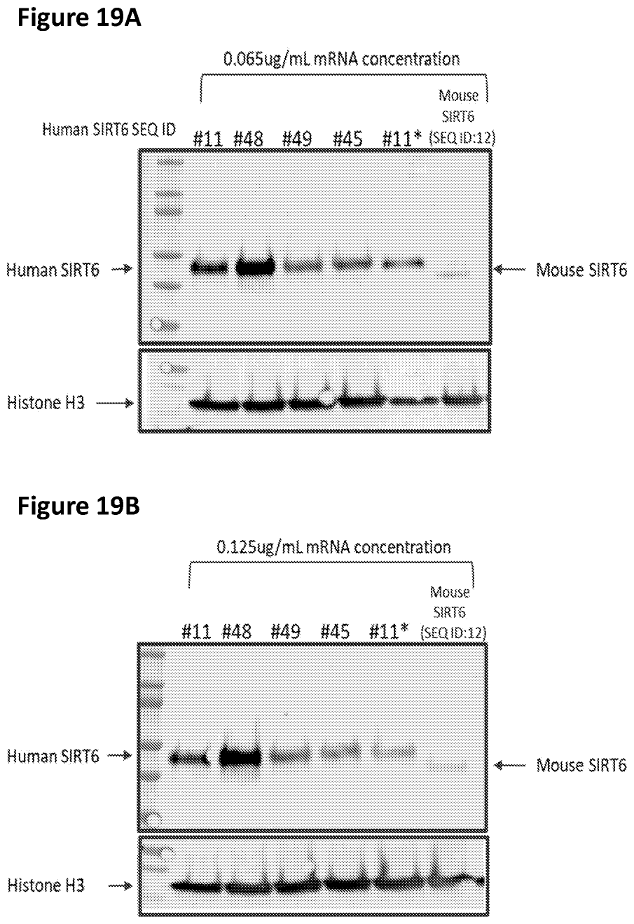

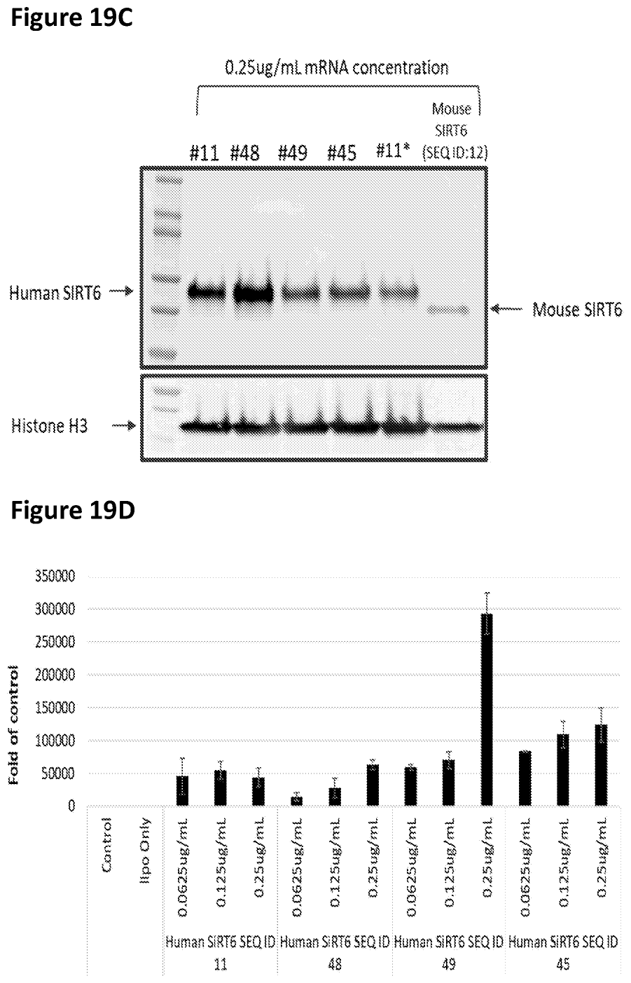

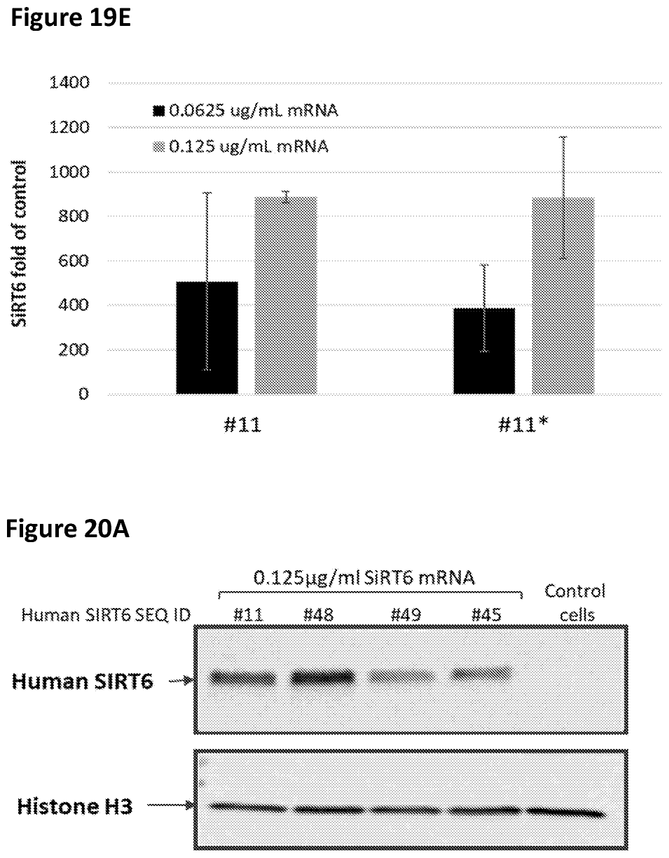

To determine whether optimizing human SIRT6 ORF codon improves mRNA translation and protein stability, human Huh7 cells were transfected with increasing doses of the following human SIRT6 mRNA (0.0625 μg, 0.125 μg, and 0.25 μg) using Lipofectamine™ MessengerMAX™ Transfection Reagent, according to manufacturer's protocol. 6 hours after transfection, medium was replaced to remove excess mRNA that did not enter the cells. Expression of SIRT6 mRNA and protein levels were evaluated 24 hours after cells transfection using RT-qPCR and Western blot analysis, respectively. Transfection of the cells resulted in increased levels of human SIRT6 protein (FIG. 19A-C), and mRNA (FIG. 19D). Specific primers matching the 3′UTR sequence of the different mRNA molecules were used to compare between the samples. Results were normalized to CycloA gene expression.

Higher expression of SiRT6 protein was seen in cells transfected with SEQ ID NO: 48 compared to the other tested sequences, as seen by the more condense signal. Loading of protein extract was similar in all samples and can be compared by the signal obtained with anti-Histone 3 antibodies. The expression level of SiRT6 protein does not correlate with the mRNA expression level. Higher mRNA levels were detected for human SiRT6 SEQ ID NOs: 49 and 45, although less protein expression is observed. On the other hand, a lower level of SiRT6 mRNA was seen in cells transfected with SEQ ID NO: 48 although these samples showed the highest expression of SiRT6 protein. These levels were higher than the native human SIRT6 ORF (SEQ ID NO: 11).

Similar results were obtained when the human SiRT6 mRNA molecules were introduced to mouse Primary hepatocytes. Mouse Primary hepatocytes were freshly isolated from C57/BL mice liver. Cells were plated in 12-well plates and 24 hours afterwards they were transfected with increasing doses (0.0625 μg, 0.125 μg, and 0.25 μg) of human SIRT6 mRNA using Lipofectamine™ MessengerMAX™ Transfection Reagent, according to manufacturer's protocol. Six hours after transfection, medium was replaced to remove excess of mRNA that did not entered the cells. Expression of SIRT6 mRNA and protein levels were evaluated 24 and 48 hours after cells transfection using RT-qPCR and Western blot analysis, respectively.

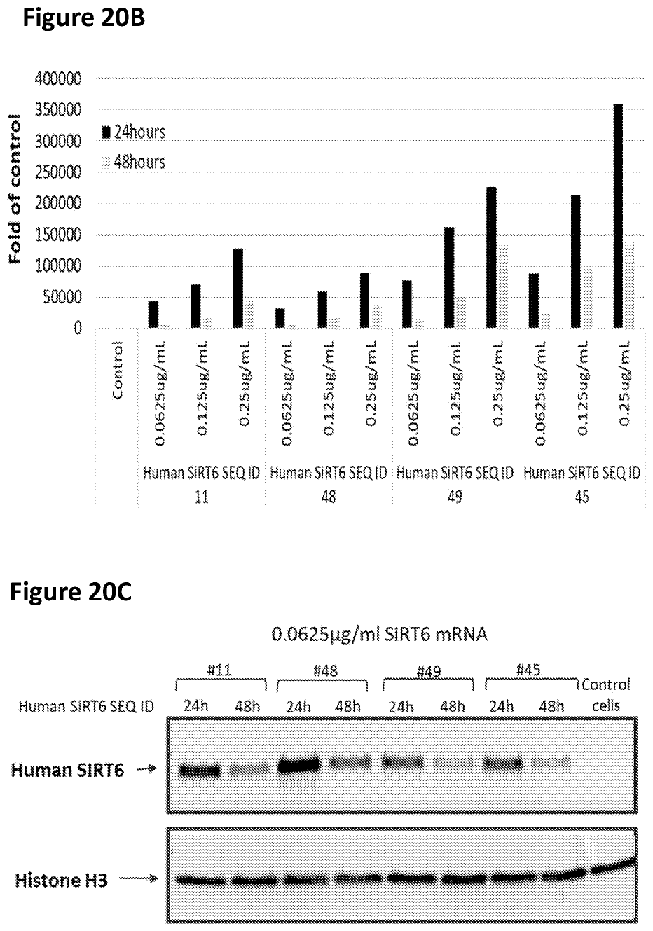

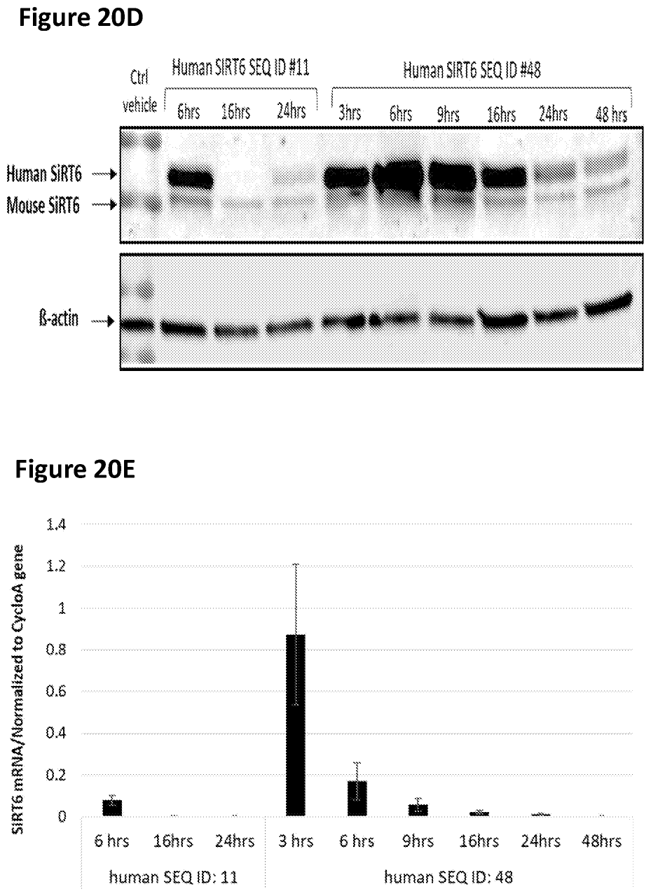

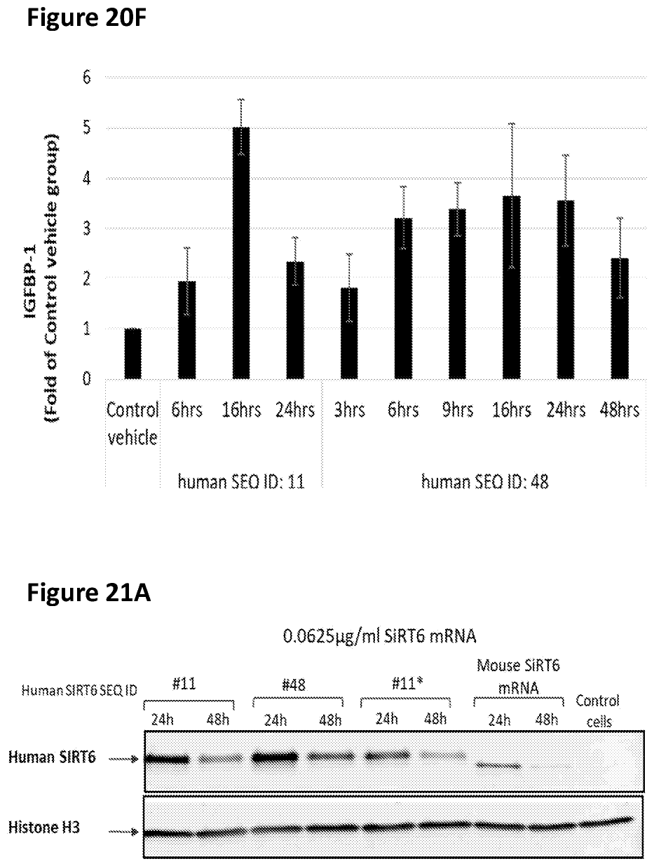

Transfection of mouse Primary hepatocytes resulted in increased levels of human SIRT6 protein (FIG. 20A), and mRNA (FIG. 20B). Like the observation in the human cell line, higher expression of SiRT6 protein was seen in cells transfected with SEQ ID NO: 48 compared to the other tested sequences, as seen by the more condense signals. This observation was similar in both tested time points, namely 24 hours and 48 hours. The loading of protein extract was similar in all samples and can be compared by the signal obtained with anti-Histone 3 Abs. Also in this experiment, the expression level of SiRT6 protein did not correlate with the mRNA expression level. Higher mRNA levels were detected for human SiRT6 SEQ ID NO:49 and SEQ ID NO:45 although less protein expression was observed. On the other hand, lower levels of SiRT6 mRNA were observed in cells transfected with SEQ ID NO: 48 although these samples showed the highest expression of SiRT6 protein. These levels were higher compared to the native human SiRT6 ORF (SEQ ID: 11).

These results show that SiRT6 mRNA codon optimization is a critical determinant in increasing protein expression and that SEQ ID NO: 48 increases human SiRT6 expression by at least two-fold compared to the wild-type (WT) sequence (SEQ ID NO: 11).

Primary hepatocytes do not divide in culture hence SiRT6 protein and mRNA stability can be followed. Both mRNA and protein levels were reduced after 48 hours in culture compared to their counterpart samples taken after 24 hours (FIG. 20C). Yet, the levels of human SiRT6 mRNA SEQ ID NO:48 is lower, but its protein level is higher compared to any of the other mRNA molecules.

Based on this data the duration of mRNA and protein expression of human SiRT6 SEQ ID NO: 48 in mice liver was evaluated. The duration of expression was compared to the human SiRT6 WT sequence (SEQ ID NO: 11).

In this experiment, human WT SIRT6 mRNA (SEQ ID NO:11) and the codon optimized human SiRT6 mRNA SEQ ID NO: 48 were formulated in D-Lin-MC3-DMA (MC3) formulation. The particles (66 nm, PDI-0.156, mRNA stock concentration 167 μg/mL) were diluted in PBS×1 to the desired concentration and injected, intravenously (iv) at an amount of 0.2 mg/kg. Mice injected with the vehicle PBS served as control. Expression of human mRNA SiRT6 and its protein levels were evaluated 6, 16 and 24 hours after injection in the livers of mice injected with the WT sequence (SEQ ID NO: 11) (N=3) and 3, 6, 9, 16, 24 and 48 hours after injection in the livers of mice injected with the codon optimized mRNA (SEQ ID NO:48) (N=3).

As shown in FIG. 20D increased expression levels of human SIRT6 protein translated from the WT mRNA transcribed from SEQ ID NO: 11 was seen at 6 hours after injection. very low SiRT6 protein level was detected in mice liver 24 hours after the injection. The human SiRT6 protein translated from SEQ ID NO:48 shows significantly improved expression of protein. Significant amounts of the protein were seen also at 16, 24 and 48 hours after injection (longer timepoints were not tested). Loading of protein extract was similar in all samples and can be compared by the signal obtained with anti β actin Abs.

The expression level of SiRT6 protein also correlated with the mRNA expression level (FIG. 20 E). Higher mRNA levels were detected for human SiRT6 SEQ ID NO: 48 compared to the WT sequence (a 2 fold, 11 fold and 6 fold increase at 6, 16 and 24 hours, respectively). These results clearly show induced mRNA and protein expression after injection of SiRT6 mRNA SEQ ID NO:48. Also, to verify the activity of the human SIRT6 protein which resulted from the injected SIRT6 mRNA, the expression levels of IGFBP1 were tested. As shown in FIG. 20F, elevation of SIRT6 protein in mice liver led to the induction of IGFBPI expression. This effect was stable for up to 48 hours (longer time points were not tested) although the levels of SiRT6 protein were reduced.

### Example 7: Improve Human SiRT6 Protein Expression by Modifying the 3′UTR Sequence

Next, the post-transcriptional role of the 3′UTR sequence on human SiRT6 translation was explored. For this purpose, the native human SiRT6 ORF sequence (SEQ ID NO: 11) was introduced by cloning into expression vectors. Each of the vectors included similar critical elements that enable efficient in vitro translation in a human cell-free system (namely they comprised the same 5′UTR including Kozak consensus sequence and a (poly (A) tail) of 80 adenosine ribonucleotides at the 3′-end) but had different 3′ UTR sequences. In this experiment, the 3′UTR sequences of mouse and human alpha-globin genes were compared since these sequences contain a C-rich stability element (CRE).

SEQ ID NO: 52:
3′ UTR derived from the human
alpha globin gene (67% GC):
GCTGGAGCCTCGGTGGCCTAGCTTCTTGCCCCTTGGGCCTCCCCC
CAGCCCCTCCTCCCCTTCCTGCACCCGTACCCCCGTGGTCTTTGA
ATAAAGTCTGAGTGGGCGGCA
Or
SEQ ID NO: 53:
3′ UTR derived from the mouse
alpha globin gene (48% GC):
GCTGCCTTCTGCGGGGCTTGCCTTCTGGCCATGCCCTTCTTCTCT
CCCTTGCACCTGTACCTCTTGGTCTTTGAATAAAGCCTGAGTAGG
AAGAAAAAAAAAAAA

a T7 bacteriophage RNA polymerase was chosen for transcription in the Human IVT System. The plasmids were designed to be suitable for Capped (Cap 1) using CleanCap® AG. Also, to improve mRNA stability, translational properties and immunogenicity, uridines were substituted with the chemically modified N1-Methylpseudouridine-5′-Triphosphate.

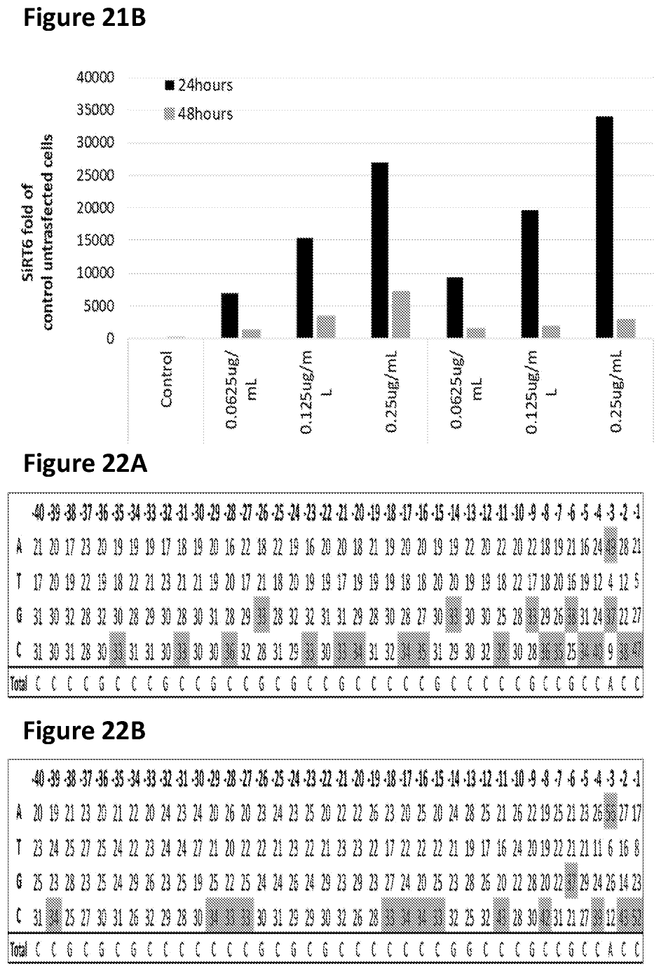

To determine the mRNA stability and translation efficiency of the different 3′UTR sequences two molecules were constructed: human SEQ ID NO:11 harboring the 3′ UTR derived from the human alpha globin gene (namely, a molecule comprising SEQ ID NO: 11 and SEQ ID NO: 52) and human SEQ ID NO:11 harboring the 3′ UTR derived from the mouse alpha globin gene (namely, a molecule comprising SEQ ID NO:11 and SEQ ID NO: 53). Human Huh7 cells or mouse primary hepatocytes were transfected with increasing doses of the human SIRT6 mRNA (0.0625 μg, 0.125 μg, and 0.25 μg) using Lipofectamine™ MessengerMAX™ Transfection Reagent, according to manufacturer's protocol. 6 hours after transfection, medium was replaced to remove excess of mRNA that did not enter the cells. Expression levels of SIRT6 mRNA and protein were evaluated 24 hours (in human Huh7 cells) or 24 and 48 hours (in mouse primary hepatocytes) after cells transfection using RT-qPCR and Western blot analysis. Similar increased levels of the two mRNA were seen in human Huh7 (FIG. 19E) and mouse primary hepatocytes (FIG. 21B) 24 hours after cells transfection. At 48 hours, the mRNA levels of human mRNA SEQ ID NO:11 with the mouse 3′UTR (designated herein for convenience: SEQ ID NO: 11*) were lower compared to the human mRNA levels of cells transfected with SEQ ID NO:11 with the human 3′UTR, probably due to reduced mRNA stability. The post-transcriptional efficiency of the mRNA (SEQ ID NO:11) with the human 3′UTR was also better than that of the same human SiRT6 sequence but with the mouse 3′ UTR (SEQ ID NO: 11*) as seen in human Huh7 cells (FIGS. 19A-C), a strong signal for SiRT6 protein in human SEQ ID NO:11 with the human 3′UTR was seen compared to SEQ ID NO:11 with the mouse 3′UTR (SEQ ID NO:11*), and in mouse primary cells, 24 and 48 hours after cells transfection (FIG. 21A). These results shows that the selection of the 3′UTR sequence affects the stability of the human SiRT6 mRNA. In the present case better performance was achieved with the 3′ UTR human alpha globin gene.

### Example 8: Improve Human SiRT6 Protein Expression by Changing the 5′UTR Sequence

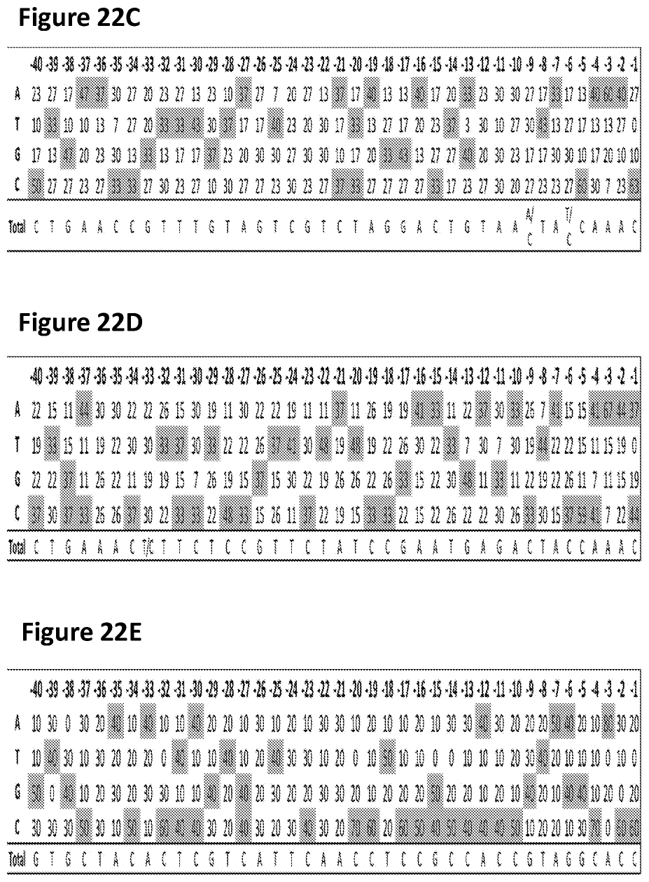

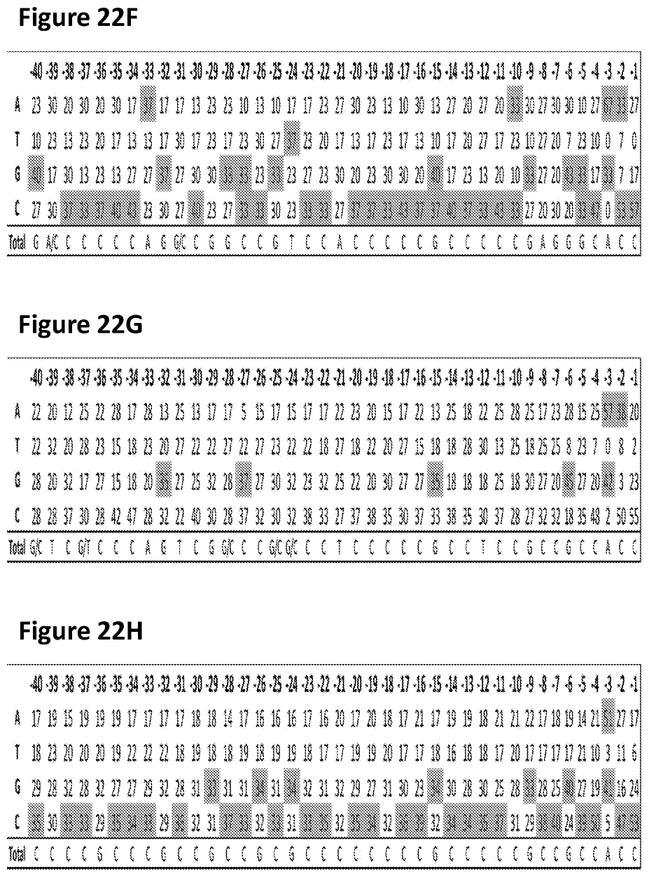

To improve the SiRT6 mRNA efficiency of translation or transcript levels, different 5′UTR sequences were explored. The master mRNA (The first transcript that was discovered) of the top 1000 genes that are elevated in Liver from the Human Protein Atlas were selected and their ORF sequences were Quired from NCBI. Out of these, 799 Top Liver genes were selected (Not all the genes in the human protein atlas list had a real gene symbol and some of the genes were with 5′ UTR of less than 40 base pairs). The 40 base pairs laying before the ORF (positions −40 t to −1) for those 799 genes were selected and analyzed to find consensus or unique 5′UTR sequences as describe below. The percentage of each of the four nucleotides (Adenine (A), Cytosine (C), Guanine (G), Thymine (T)) was calculated and manual selection was done based on the incidence of each of the four nucleotides in their position relative to the start codon as shown in FIG. 22A-H.

The selection of a consensus sequence based on the frequency of occurrence of nucleotides in the various positions created a sequence which is different in its essence from the 5′UTR sequence of other genes. Unlike other sequences, the resulted sequences are rich in GC. To explore the effect of the novel 5′UTR sequence on the regulation of SIRT6 expression, the newly designed 5′UTR consensus sequence was introduced by cloning into expression vectors. Its post transcriptional role was compared to two different known 5′UTR sequences described below:
Top Liver 5′UTR consensus sequence (TopL5′) SEQ ID NO: 54: CCCCGCCCGCCGCCGCGCCGCCCCCGCCCCCGCCGCCACC 5′UTR sequence incorporated in the Covid 19 vaccine (Vaccine5′) SEQ ID NO: 55: GAATAAACTAGTATTCTTCTGGTCCCCACAGACTCAGAGAGAACCCGCCACC

### Or

a 5′ UTR sequence used in expression vectors for expression of viral genes (also referred to herein as viral 5′ UTR or Viral5′) SEQ ID NO: 56: AGGAAATAAGAGAGAAAAGAAGAGTAAGAAGAAATATAAGAGCCACC

Each of the vectors included the wild-type sequence of the human SiRT6 gene (SEQ ID NO: 11) and similar critical elements that enable efficient in vitro translation in a human cell-free system (same 3′UTR of the human alpha globin sequence and (poly (A) tail) at the 3′-end of eukaryotic mRNAs of 120 adenosine ribonucleotides stretch).

A T7 bacteriophage RNA polymerase was chosen for transcription in the Human IVT System. The plasmids were designed to be suitable for Capped (Cap 1) using CleanCap® reagent AG (3′OMe) (and therefore AG must be presented at the beginning of each 5′UTR sequence).

To improve mRNA stability, translational properties and immunogenicity uridines were substituted with the chemically modified N1-Methylpseudouridine-5′-Triphosphate.

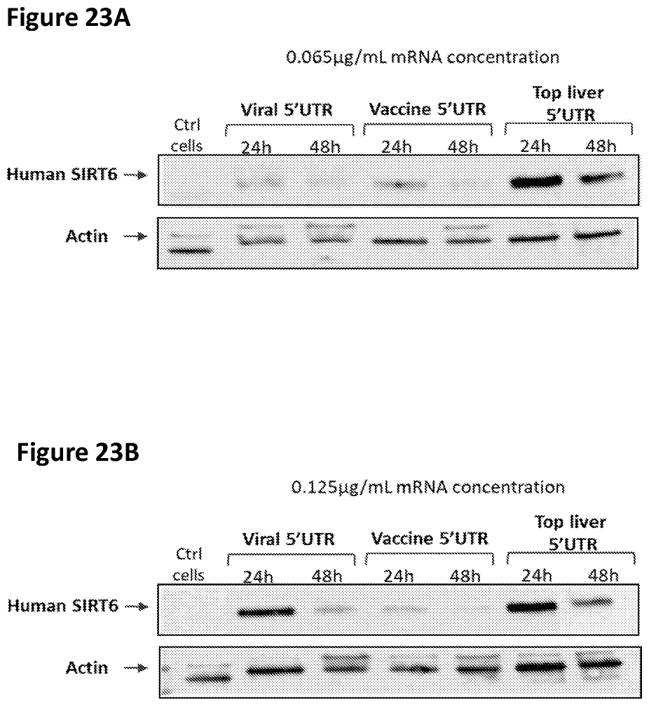

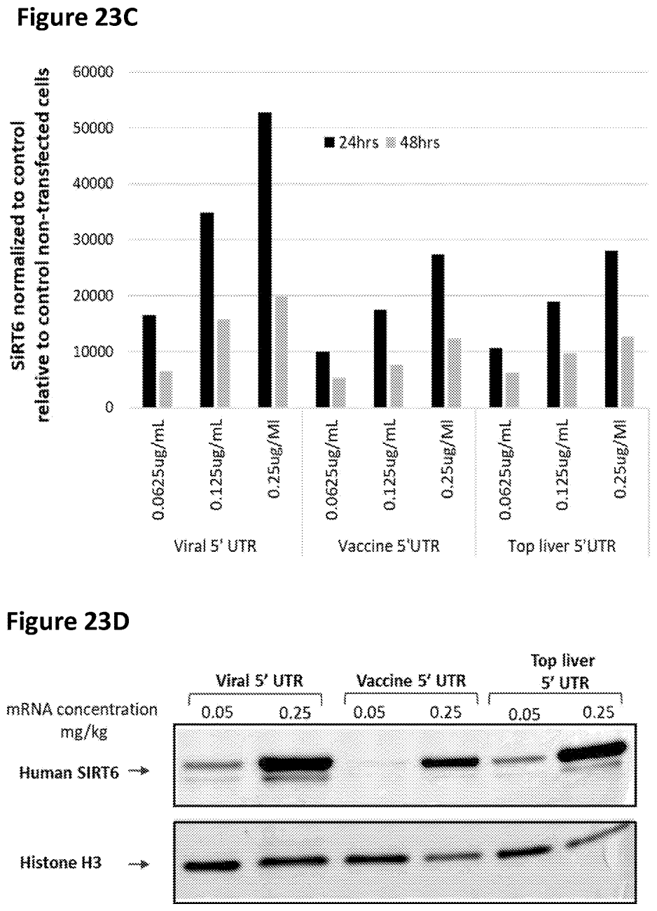

To determine the effectiveness of the novel 5′UTR sequence on human SIRT6 post translational efficiency the three different mRNA molecules (described above) having different 5′UTR sequences (TopLiver, vaccine and viral) were transfected in increasing doses (0.0625 μg, 0.125 μg, and 0.25 μg) to human Huh7 cells using Lipofectamine™ MessengerMAX™ Transfection Reagent, according to manufacturer's protocol. Six hours after transfection, medium was replaced to remove excess of mRNA that did not enter the cells. Expression levels of SIRT6 mRNA and protein were evaluated 24 and 48 hours after cells transfection using RT-qPCR and Western blot analysis. Transfection of the cells resulted in increased levels of human SIRT6 protein (FIG. 23A and 23B), and mRNA (FIG. 23C). Specific primers matching the 3′UTR sequence of the different mRNA molecules were used to compared between the samples. Results were normalized to CycloA gene expression.

The higher expression of SiRT6 protein was seen in cells transfected with the Top Liver 5′UTR mRNA sequence (SEQ ID NO: 54). The lower expression level was observed with the mRNA having the Covid 19 vaccine 5′UTR sequence. A significant change in SiRT6 expression was seen mainly in cells transfected with the lower dose of 0.0625 μg/mL in both tested timepoints, 24 and 48 hours. These results clearly demonstrate the important role of the novel 5′UTR sequence of the present invention which is significantly different from 5′UTR sequences of other genes (rich with G/C with only one A and no T) in regulating gene expression. The effect of the 5′UTR sequence on mRNA post translation is even more significant when looking at the mRNA levels (FIG. 23C). As seen in the figure, mRNA with TOP Liver 5′UTR sequences show similar amounts compared to the mRNA with COVID 19 vaccine, 5′UTR sequence. The higher mRNA amounts are seen in the mRNA with the viral 5′UTR sequence.

Similar observations were seen when the human mRNA with the different 5′ UTR sequences were formulated in D-Lin-MC3-DMA (MC3) formulation and injected to mice (FIG. 23D). The particles (around 70 nm, PDI-0.14, mRNA stock concentration 167 μg/mL) were diluted in PBS×1 to the desired concentration and injected, intravenously (iv) at escalating doses of 0.05 and 0.25 mg/kg. Mice injected with the vehicle PBS served as control. Six hours after injection (iv), liver tissues were extracted from each mouse and SIRT6 mRNA and protein levels were evaluated as described above, using Western blot analysis. As shown in FIG. 23 D, the higher expression of SiRT6 protein was seen in mice injected with the Top Liver and Viral 5′UTR mRNA sequence. The lower expression level was observed with the mRNA having the Covid 19 vaccine 5′UTR sequence.

### Discussion

The data provided herein demonstrate a profound treatment effect of SIRT6 mRNA administration in mouse models of non-alcoholic steatotic hepatitis, with marked improvement in every parameter examined. The treatment effect was observed despite the severity of the metabolic insults in the models and the initiation of treatment after accumulation of pathology.

In Example 3, for instance, improvements were observed in different markers of liver function and injury (serum AST and ALT), inflammation (MCP-1, IL-6, TNF-α, and IL-11), and fibrosis (TIMP-1 and α-SMA), and histological indicators of fat deposition, in STZ-HFD mice. These improvements were associated with profound rescue apparent in gross liver histology, as shown in FIG. 4. Similar improvements with SIRT6 mRNA treatment in these and additional markers are demonstrated in an alternative NASH mouse model in Example 5 in which a significant reduction in fibrosis markers (TIMP-1, Collagen Type-1, TR7 and TGFβ), immune cells infiltration (CD8 and F4/80) and inflammation (MCP-1 and TNF α) were observed. Example 4 demonstrates that treatment resulted in significantly increased overall survival of NASH mice, with improvements in overall phenotype readily apparent in FIG. 13.

As reviewed in the Background, SIRT6 deficiency had been associated with liver and metabolic disease and fibrosis, and SIRT6 was known to exert protective effects in the context of aging-related and metabolic diseases. However, to the inventors' knowledge, it was not heretofore known or reasonably believed that exogenous administration of SIRT6 mRNA that transiently induced SIRT6 expression in the liver is a feasible strategy for treatment of disease, including NASH. Moreover, the extent of the treatment effects observed in NASH mice, indicating rescue of liver function, phenotype, and lifespan to sub-pathological levels, was unexpected and surprising.

Treatment effects were most pronounced with high dose administration and resultant SIRT6 protein expression, strongly indicating that formulation and optimization of SIRT6 mRNA for maximal expression is advantageous for treatment of metabolic diseases, including NASH. The mRNA in the NASH mouse experiments herein (SEQ ID NO: 12) contained the wild-type murine SIRT6 protein coding region, the mouse alpha globin 3′ UTR sequence, a 120 nucleotide polyA tail, as well as a Cap1 mRNA cap, with uridine substituted for N1-Methyl-Pseudo-U throughout. The mRNA was formulated in MC3 lipid nanoparticles and administered systemically by intravenous injection. Examples 6-8 describe beneficial optimizations for human SIRT6 mRNA constructs, by optimizing SIRT6 codon sequence, replacing the 3′ UTR sequence with C-rich stability elements and new 5′ UTR sequence that was designed based on the incidence of the different nucleotides in 766 top human liver genes. And finally, introduction of the human mRNA in MC3 lipid nanoparticles (or in other lipid encapsulating agents, e.g., lipid 5 based formulation which shows significant mRNA delivery to the liver of rodends and non-human primates with improved mRNA release from the endosomes). These features significantly improve human SIRT6 protein translation and stabilization, thus facilitating treatment of diseases, including NASH, in humans.

1.-50. (canceled)

51. A non-native, isolated nucleic acid molecule encoding SIRT6, wherein said nucleic acid molecule comprises an mRNA molecule encoding SIRT6, a 3′ untranslated region (3′UTR), and a 5′ untranslated region (5′UTR).

52. The isolated nucleic acid molecule of claim 51 wherein said mRNA molecule comprises the nucleic acid sequence transcribed from SEQ ID NO: 11, or a variant thereof having at least 75%, 80%, 85%, 90%, 95% or 99% sequence identity with SEQ ID NO: 11.

53. The isolated nucleic acid molecule of claim 51, wherein said mRNA molecule encoding SIRT6 comprises a codon optimized nucleic acid sequence and wherein said codon optimized nucleic acid sequence comprises any one of the nucleic acid sequences transcribed from SEQ ID NOs: 41-49.

54. The isolated nucleic acid molecule of claim 53, wherein said codon optimized nucleic acid sequence comprises the nucleic acid sequence transcribed from SEQ ID NO:

48.

55. The isolated nucleic acid molecule of claim 51, wherein said 3′UTR is one of:
a. the native 3′UTR sequence of the SIRT6 mRNA;
b. a modified native 3′UTR sequence of the SIRT6 mRNA;
c. a heterologous 3′UTR sequence; or
d. a 3′ UTR sequence that increases the stability and translation of the SIRT6 molecule, e.g., the human alpha globin gene 3′UTR.

56. The isolated nucleic acid molecule of claim 51, wherein said 5′UTR is one of:
a. the native 5′UTR sequence of the SIRT6 mRNA;
b. a modified native 5′UTR sequence of the SIRT6 mRNA;
c. a heterologous 5′UTR sequence; or
d. a nucleic acid sequence selected from the group consisting of SEQ ID Nos: 54-56, e.g., SEQ ID No: 54.

57. The isolated nucleic acid molecule of claim 56, wherein said 5′ UTR nucleic acid sequence is a 5′UTR used in expression vectors for expression of viral genes, e.g., SEQ ID No: 56.

58. The isolated nucleic acid molecule of claim 51 further comprising a 3′ end having at least 40 consecutive adenosine (A) nucleotides, at least 80 consecutive adenosine (A) nucleotides, at least 100 consecutive adenosine (A) nucleotides, at least 120 consecutive adenosine (A) nucleotides, or about 120 consecutive adenosine (A) nucleotides.

59. The isolated nucleic acid molecule of claim 51 further comprising a 5′CAP, e.g., CAP1 AG 3′Ome.

60. The isolated nucleic acid molecule of claim 51 wherein the uridines of the mRNA molecule are substituted with N1-Methylpseudouridine-5′-Triphosphate.

61. The isolated nucleic acid molecule of claim 51, wherein said mRNA molecule encoding SIRT6 is a modified SIRT6 mRNA, and wherein said modified SIRT6 mRNA comprises one or more mutations that prevent phosphorylation and/or ubiquitination of the SIRT6 protein.

62. The isolated nucleic acid molecule of claim 61, wherein said one or more mutations is one or more of:
a. A mutation at the amino acid residue S338 (Ser338) of the SIRT6 protein;
b. A substitution of the arginine (Arg, R) and/or leucine (Leu, L) amino acid residues within the RXXL motif of the first D-box-activating domain of the SIRT6 protein with alanine (Ala, A), optionally, a substitution from RVGL to AVGA at positions 103-106;
c. A mutation in the C-terminal site of SIRT6, between amino acid residues positions 263-334; and
d. A mutation at the amino acid residue K170 (Lys170) of the SIRT6 protein, optionally a substitution of lysine to arginine (K170R).

63. The isolated nucleic acid molecule of claim 51 wherein said isolated nucleic acid molecule is encapsulated in lipid nanoparticles (LNP).

64. A method for treating a disease or disorder, for reducing frailty or for increasing longevity, in a subject in need thereof comprising a step of administering an isolated nucleic acid molecule according to claim 1 to said subject wherein said disease or disorder is at least one of a fibrotic condition, a fibrosis-associated condition (e.g., inflammation, endothelial or epithelial to mesenchymal transition, parenchymal injury, scarring, or cirrhosis), a metabolic disorder, a liver disease, a kidney disease, or cancer.

65. The method of claim 64 wherein said disease is NAFLD, e.g., nonalcoholic steatohepatitis (NASH).

66. The method of claim 65 wherein said disease is chronic kidney disease (CKD).

67. A method for reducing liver fat accumulation, liver fibrosis and/or liver inflammation in a subject in need thereof comprising a step of administering an isolated nucleic acid molecule according to claim 51 to said subject.

68. A method for reducing glucose, fat and/or triglyceride levels in the serum of a subject in need thereof comprising a step of administering an isolated nucleic acid molecule according to claim 51 to said subject.

69. The method of claim 64 wherein said method further comprises administering to said subject an additional therapeutic agent and wherein said additional therapeutic agent is an anti-miR molecule that targets a miR known to negatively regulate SIRT6 expression, or an adeno associated virus (AAV) comprising SiRT6 gene.

70. The method of claim 69, wherein said method comprises administering a single dose of the AAV followed by multiple administrations of the isolated nucleic acid molecule.


SEQUENCE LISTING (57 sequences)

SEQ ID NO: 1 - RNA, 21 nt, Homo sp.
  source 1..21: mol_type = other RNA
gtgcattgtagttgcattgca

SEQ ID NO: 2 - RNA, 20 nt, Homo sp.
  source 1..20: mol_type = other RNA
gtgcattgctgttgcattgc

SEQ ID NO: 3 - RNA, 22 nt, Homo sp.
  source 1..22: mol_type = other RNA
tggagtgtgacaatggtgtttg

SEQ ID NO: 4 - RNA, 22 nt, Homo sp.
  source 1..22: mol_type = other RNA
gcctgctggggtggaacctggt

SEQ ID NO: 5 - RNA, 21 nt, synthetic construct
  source 1..21: mol_type = other RNA
tgcaatgcaactacaatgcac

SEQ ID NO: 6 - RNA, 22 nt, synthetic construct
  source 1..22: mol_type = other RNA
caaacaccattgtcacactcca

SEQ ID NO: 7 - DNA, 20 bp, synthetic construct
  source 1..20: mol_type = other DNA
cagagctgcacggaaacatg

SEQ ID NO: 8 - DNA, 20 bp, synthetic construct
  source 1..20: mol_type = other DNA
tcatcagcgagcatcaggtc

SEQ ID NO: 9 - DNA, 20 bp, synthetic construct
  source 1..20: mol_type = other DNA
agccatgtacgtagccatcc

SEQ ID NO: 10 - DNA, 20 bp, synthetic construct
  source 1..20: mol_type = other DNA
ctctcagctgtggtggtgaa

SEQ ID NO: 11 - RNA, 1068 nt, Homo sp.
  source 1..1068: mol_type = mRNA
atgtcggtgaattacgcggcggggctgtcgccgtacgcggacaagggcaagtgcggcctcccggagatcttcgaccccccggaggagctggagcggaaggtgtgggaactggcgaggctggtctggcagtcttccagtgtggtgttccacacgggtgccggcatcagcactgcctctggcatccccgacttcaggggtccccacggagtctggaccatggaggagcgaggtctggcccccaagttcgacaccacctttgagagcgcgcggcccacgcagacccacatggcgctggtgcagctggagcgcgtgggcctcctccgcttcctggtcagccagaacgtggacgggctccatgtgcgctcaggcttccccagggacaaactggcagagctccacgggaacatgtttgtggaagaatgtgccaagtgtaagacgcagtacgtccgagacacagtcgtgggcaccatgggcctgaaggccacgggccggctctgcaccgtggctaaggcaagggggctgcgagcctgcaggggagagctgagggacaccatcctagactgggaggactccctgcccgaccgggacctggcactcgccgatgaggccagcaggaacgccgacctgtccatcacgctgggtacatcgctgcagatccggcccagcgggaacctgccgctggctaccaagcgccggggaggccgcctggtcatcgtcaacctgcagcccaccaagcacgaccgccatgctgacctccgcatccatggctacgttgacgaggtcatgacccggctcatgaagcacctggggctggagatccccgcctgggacggcccccgtgtgctggagagggcgctgccacccctgccccgcccgcccacccccaagctggagcccaaggaggaatctcccacccggatcaacggctctatccccgccggccccaagcaggagccctgcgcccagcacaacggctcagagcccgccagccccaaacgggagcggcccaccagccctgccccccacagaccccccaaaagggtgaaggccaaggcggtccccagctga

SEQ ID NO: 12 - RNA, 1005 nt, Mus musculus
  source 1..1005: mol_type = mRNA
atgtcggtgaattatgcagcagggttgtcgccttacgcggataagggcaagtgcgggctgcccgagatcttcgacccaccagaggagctggaacgcaaggtgtgggagctggcccggctaatgtggcagtcctccagcgtggttttccacacgggcgccggcatcagcaccgcctctggcatccccgacttcagaggcccccatggcgtgtggaccatggaggaacgcggcctggcccccaagtttgacaccaccttcgagaatgctcggccctcgaagacccacatggccctggttcagctagaacgcatgggcttcctcagcttcctggtcagccagaacgtagacgggctgcacgtgcgctcgggcttccccagggacaagctggcagagctgcacggaaacatgtttgtagaggaatgtcccaagtgtaagacgcagtacgtcagagacacggttgtgggcaccatgggcctcaaggccacaggccggctctgcaccgtggccaagaccaggggacttcgggcctgtagaggggagctgagagacaccattctggactgggaggactcgttgcctgaccgggacctgatgctcgctgatgaggccagcaggaccgcagacctgtctgtcaccctgggtacctcgctgcagatccgccccagtgggaacctgccccttgccactaagcgccgaggaggccgtctggtcattgtcaacctgcaacccacaaaacatgaccgccaggctgacctgcgcatccacggctacgtggatgaggtgatgtgcagactcatgaagcatctggggctggagattccagcctgggatggaccctgcgtgctagacaaagccctgccacctctgcctcgcccagtagcactcaaggctgagccccccgtgcatctcaatggtgcagtgcatgtttcgtataagtccaagcccaacagccctatactccacaggccccccaaaagagtgaagaccgaggctgcccccagctga

SEQ ID NO: 13 - DNA, 20 bp, synthetic construct
  source 1..20: mol_type = other DNA
aatggcactggtggcaagtc

SEQ ID NO: 14 - DNA, 20 bp, synthetic construct
  source 1..20: mol_type = other DNA
cagtcttggcagtgcagatg

SEQ ID NO: 15 - DNA, 20 bp, synthetic construct
  source 1..20: mol_type = other DNA
agcccagagatgacagagga

SEQ ID NO: 16 - DNA, 20 bp, synthetic construct
  source 1..20: mol_type = other DNA
gttgggctgcagctaatctc

SEQ ID NO: 17 - DNA, 20 bp, synthetic construct
  source 1..20: mol_type = other DNA
tgggttctagccagcagagt

SEQ ID NO: 18 - DNA, 20 bp, synthetic construct
  source 1..20: mol_type = other DNA
accaccagagaccgttatgc

SEQ ID NO: 19 - DNA, 20 bp, synthetic construct
  source 1..20: mol_type = other DNA
aggtccctgtcatgcttctg

SEQ ID NO: 20 - DNA, 20 bp, synthetic construct
  source 1..20: mol_type = other DNA
tctggacccattccttcttg

SEQ ID NO: 21 - DNA, 22 bp, synthetic construct
  source 1..22: mol_type = other DNA
actaccaagccagtgctgcgaa

SEQ ID NO: 22 - DNA, 22 bp, synthetic construct
  source 1..22: mol_type = other DNA
atcacaggcgaagtccaatccg

SEQ ID NO: 23 - DNA, 20 bp, synthetic construct
  source 1..20: mol_type = other DNA
cgtcagccgatttgctatct

SEQ ID NO: 24 - DNA, 20 bp, synthetic construct
  source 1..20: mol_type = other DNA
cggactccgcaaagtctaag

SEQ ID NO: 25 - DNA, 22 bp, synthetic construct
  source 1..22: mol_type = other DNA
taccacttcacaagtcggaggc

SEQ ID NO: 26 - DNA, 23 bp, synthetic construct
  source 1..23: mol_type = other DNA
ctgcaagtgcatcatcgttgttc

SEQ ID NO: 27 - DNA, 22 bp, synthetic construct
  source 1..22: mol_type = other DNA
ctgacggagatcacagtctgga

SEQ ID NO: 28 - DNA, 23 bp, synthetic construct
  source 1..23: mol_type = other DNA
ggacatcaagtctactcgaagcc

SEQ ID NO: 29 - DNA, 20 bp, synthetic construct
  source 1..20: mol_type = other DNA
ctgacagaggcaccactgaa

SEQ ID NO: 30 - DNA, 20 bp, synthetic construct
  source 1..20: mol_type = other DNA
catctccagagtccagcaca

SEQ ID NO: 31 - DNA, 20 bp, synthetic construct
  source 1..20: mol_type = other DNA
attcaaggctgtgggaaatg

SEQ ID NO: 32 - DNA, 20 bp, synthetic construct
  source 1..20: mol_type = other DNA
ctcagagtacgccagggaac

SEQ ID NO: 33 - DNA, 22 bp, synthetic construct
  source 1..22: mol_type = other DNA
caccttcctctggcacagttac

SEQ ID NO: 34 - DNA, 20 bp, synthetic construct
  source 1..20: mol_type = other DNA
tcccttcctcgatgccactt

SEQ ID NO: 35 - DNA, 20 bp, synthetic construct
  source 1..20: mol_type = other DNA
ttgcttcagctccacagaga

SEQ ID NO: 36 - DNA, 20 bp, synthetic construct
  source 1..20: mol_type = other DNA
tggttgtagagggcaaggac

SEQ ID NO: 37 - DNA, 23 bp, synthetic construct
  source 1..23: mol_type = other DNA
cgtgacagtagataccaacgctc

SEQ ID NO: 38 - DNA, 22 bp, synthetic construct
  source 1..22: mol_type = other DNA
catagcactcggcatcttctcc

SEQ ID NO: 39 - DNA, 22 bp, synthetic construct
  source 1..22: mol_type = other DNA
gcttcgtgttcaccaaggagga

SEQ ID NO: 40 - DNA, 22 bp, synthetic construct
  source 1..22: mol_type = other DNA
gtcctagaccagtgttctgagc

SEQ ID NO: 41 - RNA, 1068 nt, synthetic construct
  source 1..1068: mol_type = other RNA
atgagtgttaactatgctgccggcctgtccccttacgctgataaggggaagtgtgggctgcccgagattttcgatcccccggaagagctggaaagaaaagtgtgggagctggcgagactggtctggcagtctagctcagttgtgttccatactggagccggcatcagtacggccagcgggatcccggatttcagaggcccacacggcgtgtggacaatggaagagaggggactggcccctaaattcgatactaccttcgagagcgcgagaccaacacaaactcacatggccctggtccagctggaacgcgtggggttgctcaggttcctcgtgagtcagaacgtggacggcctgcacgtcaggagtggtttcccccgtgacaaactggccgagttgcacggcaatatgttcgtggaagagtgtgctaagtgcaaaacccagtatgtccgcgatactgtagtcggtacaatgggcctcaaggctaccggccgtctttgcactgtggctaaggcgcgcggactgagggcgtgccgcggagagctgcgcgataccatcctggactgggaggattccttgcccgacagggacctggccctggccgacgaggctagccgcaacgcggatctgtccattacactgggtacctccctgcagatccgccctagcggtaacttgcctcttgccacaaaaagacgtggagggcgtctcgtgatcgtgaatctgcagcctaccaaacacgacaggcacgcagacctcagaatccacggttacgtggacgaagtgatgactcgtctgatgaagcatttgggactggagattcctgcatgggatggcccccgtgtgctggagcgggctttgccccctctgccccgtcctccaacccccaagctggagcctaaagaggaatccccaacccgcatcaacggttctatcccagctggaccaaagcaggagccgtgtgctcaacacaacggcagcgaacccgcctcacctaagcgcgaacgccccacctccccagccccacaccgccctccaaaacgcgtgaaagccaaggcagtgccctcctga

SEQ ID NO: 42 - RNA, 1068 nt, synthetic construct
  source 1..1068: mol_type = other RNA
atgtctgtaaattacgccgccggcctctcaccatacgctgataagggcaagtgcggtcttccagagatatttgacccgcctgaggagctggagcgaaaagtctgggaactcgcacgacttgtatggcaatcctcttctgtggtatttcacaccggagccgggatctcaactgcctctggaattcccgatttccgcggtccccatggagtgtggacaatggaagaacgagggttggccccaaaatttgacaccacctttgaatccgctcggcctacacagactcacatggcgttggttcaactcgagcgggttgggttgctgagatttctcgtgagtcaaaatgttgatggtcttcatgtgagaagtgggtttccaagggacaaattggccgaactgcatgggaacatgttcgttgaggagtgcgcaaagtgcaagacacagtacgtaagggacaccgttgttgggactatgggtctgaaggctactggacgcctctgcaccgtcgcaaaggcgaggggactccgcgcttgccggggagagcttcgagatacaatccttgactgggaagattcactccctgatagagatctcgcgttggctgacgaggcatctcggaatgcagacctttccatcaccttgggtacttctcttcaaattcggccatcaggaaatcttccacttgctacaaaaagacggggtggtcgccttgtgattgtaaacctgcagcctactaaacatgatcggcacgctgatctcaggatccatggttatgttgatgaagtaatgacgcgactgatgaagcatctcggtctcgaaatacccgcttgggacgggcctagggtactcgagagagcccttcccccactgccgagaccaccgacccctaaactcgaaccaaaggaggagtccccaaccaggattaacggtagcattcccgccggacccaaacaggaaccgtgcgctcaacacaacggttcagaaccggcgtctcctaaaagagagcgaccaacctctccggcgcctcaccgcccccctaaaagggtcaaggcaaaagcagtaccttcatga

SEQ ID NO: 43 - RNA, 1068 nt, synthetic construct
  source 1..1068: mol_type = other RNA
atgagcgtgaactacgccgccggcctgagcccctacgccgacaagggcaagtgcggcctgcccgagatcttcgacccccccgaggagctggagagaaaggtgtgggagctggctagactggtgtggcagagcagcagcgtggtgttccacaccggcgccggcatctccaccgcgagcggtattcccgacttcagaggcccccacggcgtgtggaccatggaggagagaggcctggcccccaagttcgacaccaccttcgagagcgctagacccacacagacccacatggccctggtgcagctggagagagtgggcctgctgagattcctggtgtctcagaacgtggacggcctgcacgtgagaagcggcttccctagagacaagctggccgagctgcacggcaacatgttcgtggaggagtgcgccaagtgcaagacacagtacgtgagagacaccgtggtgggcaccatgggcctgaaggccaccggcagactgtgcaccgtggccaaggctagaggcctgagagcctgcagaggcgagctgagagacaccatcctggactgggaggacagcctgcccgacagagacctggccctggccgacgaggctagcagaaatgccgacttaagcattaccctgggcacaagcctgcagatcagacctagcggcaacctgcccctggccaccaagagaagaggcggcagactggtgatcgtgaacctgcagcccaccaagcacgacagacatgcagacctgagaatccacggctacgtggacgaggtgatgacaagactgatgaagcacctggggcttgagatccccgcatgggatggacctagagtgctggagagagccttaccccccttgccgagacctcccacacccaagctggagcccaaggaggagagccccacccgcatcaatggaagcatccccgctgggcccaagcaagagccctgtgctcagcacaatggcagcgagcccgctagcccgaaacgggagagaccaacgtcacctgccccccacagaccccccaagagagtgaaggccaaggccgtgcctagctga

SEQ ID NO: 44 - RNA, 1068 nt, synthetic construct
  source 1..1068: mol_type = other RNA
atgagcgtgaactatgccgctggcctgagcccctacgccgacaagggcaagtgcggcctgcctgagatctttgacccccccgaggaactggagagaaaggtgtgggagctggcccggctggtctggcagagcagcagcgtggtgttccacacaggcgccggaatcagcaccgccagcggcattcctgacttcagaggccctcacggcgtgtggaccatggaagagagaggccttgcccctaaattcgacaccacattcgagagcgccagacctacacagacacacatggccctggtccagctggagcgggtgggcctgctgcggttcctggtgtcccagaacgtggacggcctccatgtgcggagcggcttccccagagataagctggccgagctgcacggaaatatgtttgtggaagaatgtgctaagtgcaagacccagtacgtgagagatacagtggtcggcaccatgggcctgaaggccaccggcagactgtgcaccgtggccaaagccagaggactcagggcatgcagaggagagctgagagacaccatcctggactgggaagatagcctgccagatcgggacctggctctggccgatgaggcctctagaaacgccgatctgtccatcaccctgggaacatctctgcaaatcagaccctctggcaacctgcctctggctacaaagcggagaggaggcagactcgtgatcgtgaatctgcagcctaccaagcacgacagacacgccgacctgcgcatccacggctacgtggacgaggtgatgacccggctgatgaaacacctgggcctggagatccctgcctgggacggccccagagttctggaaagagctctgcctcctctgcccagacctccaacccctaaactggaacccaaggaagagagccctacgcggatcaacggcagcatcccggccggccccaagcaggagccatgtgcccagcacaacggcagcgagcctgcctccccaaagcgggaacggcctaccagccctgctcctcacagaccacctaagagggtgaaggccaaggccgtgccttcttga

SEQ ID NO: 45 - RNA, 1068 nt, synthetic construct
  source 1..1068: mol_type = other RNA
atgagcgtgaactatgccgccggactgagcccctatgccgataagggaaagtgtggcctgcctgagatcttcgaccctcctgaggaactggaaagaaaagtgtgggaactcgcccggctcgtgtggcagagtagctctgtggtgtttcacaccggcgctggcatctctacagccagcggcatccctgatttcagaggacctcatggcgtgtggaccatggaagagagaggactggccccaaagttcgacaccaccttcgagagcgccagacctacacagacacacatggccctggtgcagctggaaagagtgggactgctgagattcctggtgtcccagaatgtggacggactgcatgtgcggagcggcttccctagagataagctggccgagctgcacggcaacatgttcgtggaagagtgcgccaagtgcaagacccagtacgtgcgggatacagtcgtgggcacaatgggcctgaaagccaccggcagactgtgcactgtggccaaagctagaggcctgagagcctgtagaggcgagctgagagacaccatcctggactgggaagatagcctgccagacagagatctggccctggccgatgaggccagcagaaatgccgatctgagcatcaccctgggcaccagcctgcagatcagaccatctggcaatctgcctctggccaccaagagaagaggcggcagactcgtgatcgtgaacctgcagcctaccaagcacgacagacacgccgacctgagaatccacggctacgtggacgaagtgatgacccggctgatgaagcacctgggcctcgaaattccagcctgggatggacccagagtgctggaaagggcacttcctccactgcctagacctccaacacctaagctggaacccaaagaggaaagccccaccagaatcaacggcagcattcccgccggacctaagcaagagccttgcgctcagcacaatggcagcgaacctgctagccctaagagagagaggcctacaagccccgctcctcacagaccaccaaagagagtgaaggccaaggccgtgcctagctga

SEQ ID NO: 46 - RNA, 1068 nt, synthetic construct
  source 1..1068: mol_type = other RNA
atgagcgtgaattacgctgccggcctgagtccttatgctgacaagggcaagtgcggcctgcctgaaatcttcgaccctcctgaggagctggagcgtaaagtgtgggagctggctaggctggtgtggcagtctagctctgtggtgtttcacaccggcgccggtatctccaccgccagcggaatccctgatttcagaggcccacacggcgtctggaccatggaagagagaggattagccccaaaattcgacaccaccttcgagagtgccagacctacacagacccacatggccctggtgcagctggaaagggtcggcctgctgaggttcctggtgagccagaacgtggatggccttcatgtgaggagcggctttccaagagataagctggccgagctgcacgggaatatgttcgtggaggagtgcgccaagtgcaagacacagtacgtgagggatacagtcgtggggactatgggcctgaaagcaactggaaggctgtgcacagtggccaaggctagaggcctccgcgcctgcaggggtgagctgagagataccatcctggattgggaagactctctgcctgacagagacctggccctggctgacgaagcctctcggaatgccgatctcagcatcaccctgggaaccagcctgcagatcagaccttccggcaacctgcccctggctaccaaaagacgcggaggaagactggtgatagtgaacctgcagcctaccaagcacgacagacacgcagacctgcggatccacggctacgtggacgaggttatgactaggctgatgaaacacctgggcctggagatcccagcctgggatggacccagagtgctggagcgcgccctgccacccctgcccaggcccccaacacctaagcttgagccaaaggaggaatctccaacccggattaatggctctatcccagccggccccaagcaggagccatgtgcccagcacaacggcagtgagcccgccagtcccaagagagaacggccaacctcaccagccccacaccggccacctaaaagggtgaaagctaaagccgtgccaagctga

SEQ ID NO: 47 - RNA, 1068 nt, synthetic construct
  source 1..1068: mol_type = other RNA
atgtctgtgaattacgctgccgggctctcaccatacgcagataagggtaagtgcggtctgccagagattttcgatcctcctgaggaattggagcgaaaggtgtgggagcttgcacgcctcgtttggcagagcagcagcgtggtctttcataccggagccggaatatctactgcaagcggaatcccggatttccgaggtccacacggggtgtggaccatggaggaaagggggttggcaccaaagttcgacacaacattcgagagcgctagacctacccagactcacatggctctggtgcagctcgagagagttggcctgctccgattcctcgtgagccagaatgtggatggacttcacgtacgaagcggctttcctcgggataaactggctgagttgcacgggaacatgtttgtggaggagtgcgctaagtgtaagacccagtacgtgcgcgataccgtcgtgggcaccatgggtctcaaggcaactggcagactgtgtacagttgctaaggcacggggactgagggcatgtagaggagagctgcgcgatactatcctggactgggaagacagcctccctgatagagatctcgccctggctgacgaggcctctaggaatgccgacctctccatcacgctcggcactagcctgcagatccgcccatccggaaacctccctcttgctaccaagagaagaggaggacgacttgtgatcgtcaacctgcagccaactaagcacgacaggcacgccgatctcagaatccacgggtatgtggatgaggtcatgacacggttgatgaagcatttggggttggaaattcctgcatgggacggaccaagggtccttgagagagctctgccacctttgcctaggcctcctactcccaagttggaaccgaaagaagaatcccctactagaatcaacggctccattcctgctggacctaagcaggaaccctgtgctcagcacaatggatcagaaccagcgtcccctaaaagagagaggcccaccagccctgctcctcacagaccacctaaacgcgtgaaggctaaggctgtcccaagttga

SEQ ID NO: 48 - RNA, 1068 nt, synthetic construct
  source 1..1068: mol_type = other RNA
atgagcgtgaactacgccgccggcctgagcccctacgccgacaagggcaagtgcggcctgcccgagatcttcgacccccccgaggagctggagcgcaaggtgtgggagctggcccgcctggtgtggcagagcagcagcgtggtgttccacaccggcgccggcatcagcaccgccagcggcatccccgacttccgcggcccccacggcgtgtggaccatggaggagcgcggcctggcccccaagttcgacaccaccttcgagagcgcccgccccacccagacccacatggccctggtgcagctggagcgcgtgggcctgctgcgcttcctggtgagccagaacgtggacggcctgcacgtgcgcagcggcttcccccgcgacaagctggccgagctgcacggcaacatgttcgtggaggagtgcgccaagtgcaagacccagtacgtgcgcgacaccgtggtgggcaccatgggcctgaaggccaccggccgcctgtgcaccgtggccaaggcccgcggcctgcgcgcctgccgcggcgagctgcgcgacaccatcctggactgggaggacagcctgcccgaccgcgacctggccctggccgacgaggccagccgcaacgccgacctgagcatcaccctgggcaccagcctgcagatccgccccagcggcaacctgcccctggccaccaagcgccgcggcggccgcctggtgatcgtgaacctgcagcccaccaagcacgaccgccacgccgacctgcgcatccacggctacgtggacgaggtgatgacccgcctgatgaagcacctgggcctggagatccccgcctgggacggcccccgcgtgctggagcgcgccctgccccccctgccccgcccccccacccccaagctggagcccaaggaggagagccccacccgcatcaacggcagcatccccgccggccccaagcaggagccctgcgcccagcacaacggcagcgagcccgccagccccaagcgcgagcgccccaccagccccgccccccaccgcccccccaagcgcgtgaaggccaaggccgtgcccagctaa

SEQ ID NO: 49 - RNA, 1068 nt, synthetic construct
  source 1..1068: mol_type = other RNA
atgagcgtgaattacgccgccggcctgagcccctacgccgataagggcaagtgcggcctgcctgagatcttcgacccccctgaggagctggagagaaaagtgtgggagctggcgcggctggtgtggcagagcagcagcgtggtgttccacaccggcgccggcatctccaccgccagcggcatccctgatttcagaggcccccacggcgtgtggaccatggaagagagaggactggccccaaagttcgacaccaccttcgagagcgccagacctacacagacccacatggccctggtgcagctggagagagtgggcctgctgagattcctggtgagccagaacgtggacggcctgcatgtgcgaagcggcttccccagagacaagctggccgagctgcacggcaacatgttcgtggaggagtgcgccaagtgcaagacccagtacgtgcgagataccgtcgtgggcaccatgggcctgaaggccaccggcagactgtgcaccgtggccaaggctagaggactgagggcctgcagaggagagctgagagacaccatcctggactgggaagacagcctgcccgacagagacctggccctggccgacgaggccagcaggaatgccgacctgtccatcaccctgggcaccagcctgcagatccgaccatccggcaacctgcctctggctaccaagagacgaggaggccgactggtgatcgtgaacctgcagcctaccaagcacgacagacacgccgacctgagaatccacggctacgtggacgaggtgatgacccggctgatgaagcacctgggcctggagatccccgcctgggatggacccagagtgctggagagagccctgccacccctgcccagacctccaacccccaagctggagcccaaggaggaatcccccacccggatcaacggcagcatccccgccggacccaagcaggagccctgtgctcagcacaacggcagcgagcccgccagccctaagcgagagcggcccaccagccctgcccctcacagaccacctaaaagggtgaaggccaaggccgtccctagctga

SEQ ID NO: 50 - DNA, 19 bp, synthetic construct
  source 1..19: mol_type = other DNA
ctcggtggcctagcttctt

SEQ ID NO: 51 - DNA, 20 bp, synthetic construct
  source 1..20: mol_type = other DNA
ccgcccactcagactttatt

SEQ ID NO: 52 - RNA, 111 nt, Homo sp.
  source 1..111: mol_type = mRNA
gctggagcctcggtggcctagcttcttgccccttgggcctccccccagcccctcctccccttcctgcacccgtacccccgtggtctttgaataaagtctgagtgggcggca

SEQ ID NO: 53 - RNA, 105 nt, Mus musculus
  source 1..105: mol_type = mRNA
gctgccttctgcggggcttgccttctggccatgcccttcttctctcccttgcacctgtacctcttggtctttgaataaagcctgagtaggaagaaaaaaaaaaaa

SEQ ID NO: 54 - RNA, 40 nt, synthetic construct
  source 1..40: mol_type = other RNA
ccccgcccgccgccgcgccgcccccgcccccgccgccacc

SEQ ID NO: 55 - RNA, 52 nt, synthetic construct
  source 1..52: mol_type = other RNA
gaataaactagtattcttctggtccccacagactcagagagaacccgccacc

SEQ ID NO: 56 - RNA, 47 nt, Vir sp.
  source 1..47: mol_type = mRNA
aggaaataagagagaaaagaagagtaagaagaaatataagagccacc

SEQ ID NO: 57 - RNA, 508 nt, Homo sp.
  source 1..508: mol_type = mRNA
ccagggtgcttggggagggtggggctttttgtagaaactgtggattctttttctctcgtggtctcactttgttacttgtttctgtccccgggagcctcagggctctgagagctgtgctccaggccaggggttacacctgccctccgtggtccctccctgggctccaggggcctctggtgcggttccgggaagaagccacaccccagaggtgacaggtgagcccctgccacaccccagcctctgacttgctgtgttgtccagaggtgaggctgggccctccctggtctccagcttaaacaggagtgaactccctctgtccccagggcctcccttctgggccccctacagcccaccctacccctcctccatgggccctgcaggaggggagacccaccttgaagtgggggatcagtagaggcttgcactgcctttggggctggagggagacgtgggtccaccaggcttctggaaaagtcctcaatgcaataaaaacaatttctttcttgca